SEQUENCE LISTING (442 sequences)

SEQ ID NO: 1 - DNA, 60 bp, Artificial sequence
  probe
caaggatcccctcgagactactctgttaccagtcatgaaacattaaaacctacaagcctt

SEQ ID NO: 2 - DNA, 60 bp, Artificial sequence
  probe
ccccccataaagacaaaccaatcaaccacgacaaaggaagttgacctaaacatgtaacca

SEQ ID NO: 3 - DNA, 60 bp, Artificial sequence
  probe
acccatagggagctgatcgtaatgtttatcatgttacttccccacccctacattttttga

SEQ ID NO: 4 - DNA, 60 bp, Artificial sequence
  probe
ccccttgatagttcacaagagaagtcgtttcattcaagttggtgtaatcagctggggagt

SEQ ID NO: 5 - DNA, 60 bp, Artificial sequence
  probe
acgtaaaagagtggttagtcacgtgaattcagttatcatttgacagattcttatctgtac

SEQ ID NO: 6 - DNA, 60 bp, Artificial sequence
  probe
aaataggtggtagatattgaggccaagaatattgcaaaatacatgaagcttcatgcactt

SEQ ID NO: 7 - DNA, 60 bp, Artificial sequence
  probe
actgaagaagggatgtccgctatatccaaaattacagctattggcaaataaacgagatgg

SEQ ID NO: 8 - DNA, 60 bp, Artificial sequence
  probe
gcttggtaataagaagcacttaaatcactccaaagaagactttaaaaagggagcagtgaa

SEQ ID NO: 9 - DNA, 60 bp, Artificial sequence
  probe
cagatggcaagattgagtttatttcaacaatggaaggatataagtatccagtatatggtg

SEQ ID NO: 10 - DNA, 60 bp, Artificial sequence
  probe
ctggctcatagcatttcacaaacattatacttcagagtcccaaagcctttaaataaaatg

SEQ ID NO: 11 - DNA, 60 bp, Artificial sequence
  probe
tattttatcagcactttatgcacgtattattgacattaatacctaatcggcgagtgccca

SEQ ID NO: 12 - DNA, 60 bp, Artificial sequence
  probe
gagcaccatgtgtcatcacagacacttacacatacttgaaacttggaataaaagaaagat

SEQ ID NO: 13 - DNA, 60 bp, Artificial sequence
  probe
ttgctctatcattctgtttcaataaagacatttggaataaacgagcatatcatagcctgg

SEQ ID NO: 14 - DNA, 60 bp, Artificial sequence
  probe
ggtacattactgcaatgttctcttaacagttaaacaagctgtttacagtttaaactgctg

SEQ ID NO: 15 - DNA, 60 bp, Artificial sequence
  probe
cagcaacctcagaccaacccaagaagataatttaaatctatactgcttattggtcaatat

SEQ ID NO: 16 - DNA, 60 bp, Artificial sequence
  probe
gtcactctacatgaattatgtgctcaaatttgaccaactcagtttaagacacaaaacagt

SEQ ID NO: 17 - DNA, 60 bp, Artificial sequence
  probe
ggagggtggtcaacaaagaaacaaagatgttatggtgtttagacttatggttgttaaaaa

SEQ ID NO: 18 - DNA, 60 bp, Artificial sequence
  probe
ggcagtttacagaactcaatgacttgtcatgaggttttcatatgagctacacattgtgta

SEQ ID NO: 19 - DNA, 60 bp, Artificial sequence
  probe
tccccaggctataaatttgttctcacaaagcaacatcaataaatcaaaactgtctctccc

SEQ ID NO: 20 - DNA, 60 bp, Artificial sequence
  probe
aataatgctaccaggatgctaaagatgatgatgttaacccattccagtacagtattcttt

SEQ ID NO: 21 - DNA, 60 bp, Artificial sequence
  probe
gaaagagaaacttgtcaactcatatccacgttatctagcaaagtcataagaatctatcac

SEQ ID NO: 22 - DNA, 60 bp, Artificial sequence
  probe
tcatcagttctgcgaaggagatggttggtcagaagatgaagtacagtattgtgagcagga

SEQ ID NO: 23 - DNA, 60 bp, Artificial sequence
  probe
gttgcaatctatgccgtgtatttttgctttaagtcagaaccttggattacaaaacctcga

SEQ ID NO: 24 - DNA, 60 bp, Artificial sequence
  probe
atcaaacaaaatccttgaggttggtatacaagttaaggctgaaaaaaggccttaaattcc

SEQ ID NO: 25 - DNA, 60 bp, Artificial sequence
  probe
tttttatactgattggttcatagatggtcagttttgtacacagactgaacaatacagcac

SEQ ID NO: 26 - DNA, 60 bp, Artificial sequence
  probe
gtattaataacgtctttttatatctaaatgtattctgcacgagaaggtacactggtccca

SEQ ID NO: 27 - DNA, 60 bp, Artificial sequence
  probe
tgtaggaaagtaaaatggaaggaaattgggtgcatttctaggacttttctaacatatgtc

SEQ ID NO: 28 - DNA, 60 bp, Artificial sequence
  probe
tatgcatgtaatggactatgcacacttttaattttgtcagattcacacatgccactatga

SEQ ID NO: 29 - DNA, 60 bp, Artificial sequence
  probe
ctaaaaggccacccctttatcgcattgctgtccttgggtagaatataaaataaagggact

SEQ ID NO: 30 - DNA, 60 bp, Artificial sequence
  probe
tttatacccgtttcacatgcttttcaagaatgtcgcaattactaagaagcagataatggt

SEQ ID NO: 31 - DNA, 60 bp, Artificial sequence
  probe
gtaccgcttccttagaactctacagaagccaagctccctggaagccctgttggcagctct

SEQ ID NO: 32 - DNA, 60 bp, Artificial sequence
  probe
atggtggtctgctgtgtgaataaataattcctgaagaatgaagaagattaattttgggag

SEQ ID NO: 33 - DNA, 60 bp, Artificial sequence
  probe
agcaaacaattagctgaaaacttggtattgttgtagtttatgtagtaagtgacttggcac

SEQ ID NO: 34 - DNA, 60 bp, Artificial sequence
  probe
gcacactgtcaaattcttttccttaaggtgcacagtaaatgtacagatagttataggcca

SEQ ID NO: 35 - DNA, 60 bp, Artificial sequence
  probe
caaaaagacatggaaggttaggtttgtcaacacaaagacatggaagattagaggtttgtc

SEQ ID NO: 36 - DNA, 60 bp, Artificial sequence
  probe
ttggtacgattcctgtcatccctattgcagagtcgtgtccccaataaacaggtgtcttaa

SEQ ID NO: 37 - DNA, 60 bp, Artificial sequence
  probe
ttgggtctttgataaagaaaagaatccctgttcattgtaagcacttttacggggcgggtg

SEQ ID NO: 38 - DNA, 60 bp, Artificial sequence
  probe
attccttaagaactctgtgttatattaccatggaacgcctaataaagcaaaatgtggttg

SEQ ID NO: 39 - DNA, 60 bp, Artificial sequence
  probe
taatagccttgtttgtgttagcaaagtggaatctatcagcattgttgaaatgcttaagac

SEQ ID NO: 40 - DNA, 60 bp, Artificial sequence
  probe
cacaagaagaaagtatcgggacattattggatgctatcatttgtagaatgtcaacaaaag

SEQ ID NO: 41 - DNA, 60 bp, Artificial sequence
  probe
agctctgacatcccttcctggaaacagcatgaataaaacactcatcccatgggtccaaat

SEQ ID NO: 42 - DNA, 60 bp, Artificial sequence
  probe
tgtgctatcgaaagtatgcatgtattttacgtacaaggttcgctacactaacagctccac

SEQ ID NO: 43 - DNA, 60 bp, Artificial sequence
  probe
ccgagatatctgttttgcaacaagatgggctatatctaaataaagacatgatcaaaggaa

SEQ ID NO: 44 - DNA, 60 bp, Artificial sequence
  probe
gctattacgaagttcaagatcaaaaaggcttataaaacagagtaatcttgttggttcacc

SEQ ID NO: 45 - DNA, 60 bp, Artificial sequence
  probe
gtgtttcatatgcgtgaccgttaagatattatcatttaggtgaaggtttcaactcaaaac

SEQ ID NO: 46 - DNA, 60 bp, Artificial sequence
  probe
agacctaactgttaccatctgactgaggttactctggtatttatctcttcgctgctgaaa

SEQ ID NO: 47 - DNA, 60 bp, Artificial sequence
  probe
tctcttgcaacacaaatggttagaatgattttgaagattgatgacattcgtaagcctgga

SEQ ID NO: 48 - DNA, 60 bp, Artificial sequence
  probe
cggagaggtcagtaactaaattggagaaaagcattgatgacttagaagacgagctgtacg

SEQ ID NO: 49 - DNA, 60 bp, Artificial sequence
  probe
aaggagactttcttaagcaccatatagatagggttatgtataaaagcatatgtgctactc

SEQ ID NO: 50 - DNA, 60 bp, Artificial sequence
  probe
atgtgctaaactggaggtaactatttctaggtagttgaatttttgaaagtcatgatcagc

SEQ ID NO: 51 - DNA, 60 bp, Artificial sequence
  probe
tataattactttagctgcactaacagtacaatgcttgttaatggttaatataggcagggc

SEQ ID NO: 52 - DNA, 60 bp, Artificial sequence
  probe
agctctagcttttgcagtcgtgtaatgggcccaagtcattgtttttctcgcctcactttc

SEQ ID NO: 53 - DNA, 60 bp, Artificial sequence
  probe
gactcagttttcaattccatcctaaaactccttttaaccaagcttagcttctcaaaggcc

SEQ ID NO: 54 - DNA, 60 bp, Artificial sequence
  probe
gagagagaaaacatacccacagataaagactcagaatttacttctttgggtatgagtgcc

SEQ ID NO: 55 - DNA, 60 bp, Artificial sequence
  probe
gcggatagaaggctgtatcattggttttgatgagtatatgaaccttgtattagatgatgc

SEQ ID NO: 56 - DNA, 60 bp, Artificial sequence
  probe
taggagaaccaaatctggaagccaaaatgtaggcttagtttgtgtgttgtctcttgagtt

SEQ ID NO: 57 - DNA, 60 bp, Artificial sequence
  probe
caaggtgaagatctaaatgtgaacagtttactaatgcactactgaagtttaaatctgtgg

SEQ ID NO: 58 - DNA, 60 bp, Artificial sequence
  probe
gcagcaacaataaatagacacgcacggcagccacagcttgggtgtgtgttcatccttgtt

SEQ ID NO: 59 - DNA, 60 bp, Artificial sequence
  probe
gcattgctgtgaattagctcacttggtgatatgtcctatattggctaaattgaaacctgg

SEQ ID NO: 60 - DNA, 60 bp, Artificial sequence
  probe
gatggcccagcctgtaagatactgtatatgcgctgctgtagataccggaatgaattttct

SEQ ID NO: 61 - DNA, 60 bp, Artificial sequence
  probe
gcagccgtggtagtagctgtctatgattcttgctcagcaaagtaaaataaatgttaaata

SEQ ID NO: 62 - DNA, 60 bp, Artificial sequence
  probe
cagaggaggagtattccaccaaagaagataaatatgaagaggagatcaaactgttggagg

SEQ ID NO: 63 - DNA, 60 bp, Artificial sequence
  probe
ataattctctgtacaggggggtttgtgctatacactgggatgtctaattgcagcaataaa

SEQ ID NO: 64 - DNA, 60 bp, Artificial sequence
  probe
aggaagaagacacattcctagttccccgtgaacttcctttgacttattgtccccactaaa

SEQ ID NO: 65 - DNA, 60 bp, Artificial sequence
  probe
ttcatttcaaaggtgctaaaatctgaaatctgctagtgtgaaacttgctctactctctga

SEQ ID NO: 66 - DNA, 60 bp, Artificial sequence
  probe
tgcgcagcacttaaccagtcgtttgtattccctttctttcaatccaataaacagaatgaa

SEQ ID NO: 67 - DNA, 60 bp, Artificial sequence
  probe
gtctgaaattcatgcaagcttccgaagatcttctcaaggaacactacgttgacctgaagg

SEQ ID NO: 68 - DNA, 60 bp, Artificial sequence
  probe
gaaaatacattagcaagcttaatggttatccttacttgagtccacatgggttggacagtc

SEQ ID NO: 69 - DNA, 60 bp, Artificial sequence
  probe
aagctgtgctgtttagaaacaacatctcagactttacaaagaaatgacaaagaaggcaat

SEQ ID NO: 70 - DNA, 60 bp, Artificial sequence
  probe
accccttttaagaatttggcacagttactcactttgtgtaatctgaaatctagctgctga

SEQ ID NO: 71 - DNA, 60 bp, Artificial sequence
  probe
ctggttacgaagaaaactcaccaatcatctccttcctgttaatcacatgttaatgaaaat

SEQ ID NO: 72 - DNA, 60 bp, Artificial sequence
  probe
atacctcattctgacacctgcatatagtgtgggaaattgctctgcatttgacttaattaa

SEQ ID NO: 73 - DNA, 60 bp, Artificial sequence
  probe
ccgtggagatcccaactggtttatgaagaaagcgcaggagcataagagggaattcacaga

SEQ ID NO: 74 - DNA, 60 bp, Artificial sequence
  probe
agacgtgcctttaagcaataaaaattccaagagctgatcattattgtgcttccattttag

SEQ ID NO: 75 - DNA, 60 bp, Artificial sequence
  probe
gaaacatcatgtcacatgaaacgattctctgctttttggttctgaacttgaagtccctaa

SEQ ID NO: 76 - DNA, 60 bp, Artificial sequence
  probe
ttgacttcaatgatagtaagaaaagtggttgttagttatagatgtctaggtacttcaggg

SEQ ID NO: 77 - DNA, 60 bp, Artificial sequence
  probe
tctgcctcagtttcccctcctaatacatatggctgttttccacctcgataatataacacg

SEQ ID NO: 78 - DNA, 60 bp, Artificial sequence
  probe
ccgacaagaggagatcattttagatattaccgaaatgaagaaagcttgcaattagtgaac

SEQ ID NO: 79 - DNA, 60 bp, Artificial sequence
  probe
gattccagctatgtaacctctatgctctgtaaggtgattatttgtatatagcaacatggc

SEQ ID NO: 80 - DNA, 60 bp, Artificial sequence
  probe
atccaaaaatagccctgcaaaaattcagagtccttgcaaaattgtctaaaatgtcagtgt

SEQ ID NO: 81 - DNA, 60 bp, Artificial sequence
  probe
gaggttttggttccactggaaagaattaaaatttatgccaagaacagaaaacaagaagtc

SEQ ID NO: 82 - DNA, 60 bp, Artificial sequence
  probe
gtgatttccagtgagtgtaaataattttaaataacctctggcccttggaataaagttctg

SEQ ID NO: 83 - DNA, 60 bp, Artificial sequence
  probe
ttccactcctgaaatgagttgatttgcagataactcacaacttcttaagctaaatggtat

SEQ ID NO: 84 - DNA, 60 bp, Artificial sequence
  probe
atttttggagtcccattgtttcagtgggcattaacagaatgctttaaaaacttctaagac

SEQ ID NO: 85 - DNA, 60 bp, Artificial sequence
  probe
cgagtcttaaccatatttaagtgttactgtggcttcaaagaagctattgattctgaagta

SEQ ID NO: 86 - DNA, 60 bp, Artificial sequence
  probe
ggcaatcattttcagaccactatgtgtttgaatcctctggtatcaatacgtattataggg

SEQ ID NO: 87 - DNA, 60 bp, Artificial sequence
  probe
gcctgcaaacacatttacttctcctcttatgagactatttatctttaataaagcactggg

SEQ ID NO: 88 - DNA, 60 bp, Artificial sequence
  probe
gagtcatgagaaccatcagttcttgatattgtctagacttgcatctagagctacgttgta

SEQ ID NO: 89 - DNA, 60 bp, Artificial sequence
  probe
aaggaattttcaatatttgattggtattctgttctgaagtctaggatatttttcagccta

SEQ ID NO: 90 - DNA, 60 bp, Artificial sequence
  probe
tatactgacgaagccattgaaacagatgatctgaccattaagctggtgatcaaggcactc

SEQ ID NO: 91 - DNA, 60 bp, Artificial sequence
  probe
attttacactgtttggaagaaagctaaaccctgaagatcagtagcccctaatcacatgtg

SEQ ID NO: 92 - DNA, 60 bp, Artificial sequence
  probe
gaattcacctaaagatcaaaatatcatggattgaacctcatcaattgatagcagtgagtg

SEQ ID NO: 93 - DNA, 60 bp, Artificial sequence
  probe
taaaagtaaccattggaaacctcgaatgagggctaaagttttaatcataagagaaaaggc

SEQ ID NO: 94 - DNA, 60 bp, Artificial sequence
  probe
aaatgtggagaagataacacttgattccatttcattgtcattagtgtattaaccagcagg

SEQ ID NO: 95 - DNA, 60 bp, Artificial sequence
  probe
gatattgttaatataataataattattttttagagtactgcttttgtatgtatgttgaac

SEQ ID NO: 96 - DNA, 60 bp, Artificial sequence
  probe
tgcagcatttgataatgataaaacacctcacacctcactctttatagtgcacaaaatgaa

SEQ ID NO: 97 - DNA, 60 bp, Artificial sequence
  probe
gttggcattgatatggtacaacctgcaaattacttgcagttctgagtttcagataaaaca

SEQ ID NO: 98 - DNA, 60 bp, Artificial sequence
  probe
gtaatgcttctaaggatctcaaagtaaagcgtatcactccgcgtcacttgcagcttgcaa

SEQ ID NO: 99 - DNA, 60 bp, Artificial sequence
  probe
attcatgctcatcgtatctgtgttggcactgataccagaaaccacaacattgacagttgg

SEQ ID NO: 100 - DNA, 60 bp, Artificial sequence
  probe
tgaacagttgcaaagatgtcttaattgtgtaaagaattggtgtagtcatgactttagctg

SEQ ID NO: 101 - DNA, 60 bp, Artificial sequence
  probe
caaagcactgtgtggctgaataagcggtgttcatgatttcttttattctttgcacaacaa

SEQ ID NO: 102 - DNA, 60 bp, Artificial sequence
  probe
catctcctcggtataaatcatgtttataagttatggaagaaccgggacattttacagaaa

SEQ ID NO: 103 - DNA, 60 bp, Artificial sequence
  probe
tgggcactgtggttttatttcctaattgatttaagaaataaacctgaagaccgtctggtg

SEQ ID NO: 104 - DNA, 60 bp, Artificial sequence
  probe
ttaccacctatgactgtacttgtcagtatgagaatttttctgaatcatattggggaagca

SEQ ID NO: 105 - DNA, 60 bp, Artificial sequence
  probe
gttttgccacagttaacccattgtgcttctttgtaatcaaacagtttgtgggagaatggg

SEQ ID NO: 106 - DNA, 60 bp, Artificial sequence
  probe
gagaactagatagcattgaagctgaacttacaagaagagtagacatgatggaactgtgac

SEQ ID NO: 107 - DNA, 60 bp, Artificial sequence
  probe
catctcattgtacagtgttttagttgcaagcagaaagtagaatttggtataaagcaggtt

SEQ ID NO: 108 - DNA, 60 bp, Artificial sequence
  probe
gagacaagaggaaagaaacaaagcatttattccacctaaggaaaaaccaattgtgaaacc

SEQ ID NO: 109 - DNA, 60 bp, Artificial sequence
  probe
cctttgtgttaatatcagaagtgtatttgtagcccctccatagtgaacaatgaaataaac

SEQ ID NO: 110 - DNA, 60 bp, Artificial sequence
  probe
aaacccatgaaggtaactaacggaaggaaaaactaagagaatgaaaagtatttgcctctg

SEQ ID NO: 111 - DNA, 60 bp, Artificial sequence
  probe
atatgtaccaacaacacgaattgcacagtcatcaatgacagctttcaagagtactccacg

SEQ ID NO: 112 - DNA, 60 bp, Artificial sequence
  probe
tgatttggggggaaattaccagtagaatgccttggtctgaatatttgatagaaccaattg

SEQ ID NO: 113 - DNA, 60 bp, Artificial sequence
  probe
acagacaccagatttgtgaataaagttggggaatggacagcctaactgggacattgcagt

SEQ ID NO: 114 - DNA, 60 bp, Artificial sequence
  probe
tgtggagaatgatctgtacatagattttggtggaaagtttcattgtgtatgtagaagacc

SEQ ID NO: 115 - DNA, 60 bp, Artificial sequence
  probe
ctgaaacagaaatgaaactgtccttttgacaactctcttatataataaagtatcaccggc

SEQ ID NO: 116 - DNA, 60 bp, Artificial sequence
  probe
tcatacaatttaattgctcaaccatgcatttaaaactcctcaagaaaggattggtactgc

SEQ ID NO: 117 - DNA, 60 bp, Artificial sequence
  probe
gccagattgccttcatcatttcacctctccagacttccatttcttcacaaggatgatgat

SEQ ID NO: 118 - DNA, 60 bp, Artificial sequence
  probe
atgctgcaggttgatgtagacacagtaaaaggagggctgaagttgaaccccaactgcctg

SEQ ID NO: 119 - DNA, 60 bp, Artificial sequence
  probe
ctgtgactttaataagctggaacagtccactgaatgggtataatgaattgcagtatatac

SEQ ID NO: 120 - DNA, 60 bp, Artificial sequence
  probe
aggttctgtcagttcttatcaaaaagctcggtactgcactacaggatgaaaaggaaaaga

SEQ ID NO: 121 - DNA, 60 bp, Artificial sequence
  probe
ctttgctttgattgaaggctgtagagctgagttaccaaaatttctatttcaaaggaaacc

SEQ ID NO: 122 - DNA, 60 bp, Artificial sequence
  probe
gagaatcaaataatagatgtccgtacaagtagcgcatatatttaaccatttagtttgggg

SEQ ID NO: 123 - DNA, 60 bp, Artificial sequence
  probe
gtcacacaccagcactttatacacttctggctcacaggaaagtgtctgcagtagggaccc

SEQ ID NO: 124 - DNA, 60 bp, Artificial sequence
  probe
agtataatctgctttacaactagtatagacctaaggtcatttgctttcaattagaggctc

SEQ ID NO: 125 - DNA, 60 bp, Artificial sequence
  probe
gcaagaactctgggcttgggtaatgagcaggaagaaaattttctgatcttaagcccagct

SEQ ID NO: 126 - DNA, 60 bp, Artificial sequence
  probe
aagagaccatggaagtgtcagagattcagaatccaagattgtctttaagttttcaactgt

SEQ ID NO: 127 - DNA, 60 bp, Artificial sequence
  probe
ccatgagtaactctgacaggtattttagatcatgatctcaacaatattcttccaaaatgg

SEQ ID NO: 128 - DNA, 60 bp, Artificial sequence
  probe
ggcagacacggatctgagaatctttattgagaaagagcacttaagagaatattttaagta

SEQ ID NO: 129 - DNA, 60 bp, Artificial sequence
  probe
agacagatatgtaccttattagagcaccagaactaatttgctaagtcttttgtttagtcc

SEQ ID NO: 130 - DNA, 60 bp, Artificial sequence
  probe
tagacttgaatctactctaaacgaatatttaatccaacctcactacattgtagctcagtc

SEQ ID NO: 131 - DNA, 60 bp, Artificial sequence
  probe
acatacacagatcctaagtagaaccaggtaattgtctctttttctaataaggaatttggg

SEQ ID NO: 132 - DNA, 60 bp, Artificial sequence
  probe
taaagtgtcaagtgattaagtgtgtatttgtaccctagatgatatgaaccagcagtcttg

SEQ ID NO: 133 - DNA, 60 bp, Artificial sequence
  probe
gcatcgtgtacttaccagtgtgttcacaaaatgaaatttgtgtgagagctgtacattaaa

SEQ ID NO: 134 - DNA, 60 bp, Artificial sequence
  probe
cctaaatgaaagtgtgtaaattataagaagctggcgatcttttgatatgctgtttcacag

SEQ ID NO: 135 - DNA, 60 bp, Artificial sequence
  probe
accatgtaaaatgttgtagagatagagccatataacgtcacgtttcaaaactagctctac

SEQ ID NO: 136 - DNA, 60 bp, Artificial sequence
  probe
gagtcagagtctttttatttaggccagttgtcaagtgtcaataaaagcgcatcatgtaat

SEQ ID NO: 137 - DNA, 60 bp, Artificial sequence
  probe
actggttgatgatagattttataacctaacggttctcatgcggtgcgtaattgtagatgc

SEQ ID NO: 138 - DNA, 60 bp, Artificial sequence
  probe
cgttcaggcttactcagagatttgattgctcaacatgcataacttgaattcaatagactt

SEQ ID NO: 139 - DNA, 60 bp, Artificial sequence
  probe
atgcagaagctacagatacagaggctacagaaacataaatgagctgactttagtgagcat

SEQ ID NO: 140 - DNA, 60 bp, Artificial sequence
  probe
ctgggttgatacctgaaagaatcctgtcttatttggtctccataatcctttgaatggaaa

SEQ ID NO: 141 - DNA, 60 bp, Artificial sequence
  probe
cactacacagtctgcaagattctgaaacattgctttgaccactcttcctgagttcagtgg

SEQ ID NO: 142 - DNA, 60 bp, Artificial sequence
  probe
catgaccagccacatctgaaaaggaaagcattctgctctttccagatctacgctgtgcct

SEQ ID NO: 143 - DNA, 60 bp, Artificial sequence
  probe
gtgaacaggcatgttgtattctataacacaatcttgagcattcttgacagactcctggct

SEQ ID NO: 144 - DNA, 60 bp, Artificial sequence
  probe
ttgacgagtatcatagtggtcatattgatagagcttttgatatcattgagcgcttgaagc

SEQ ID NO: 145 - DNA, 60 bp, Artificial sequence
  probe
atattacctgttctgcttcaccaggagatcatgctgctgtgatactgagttttctaaaca

SEQ ID NO: 146 - DNA, 60 bp, Artificial sequence
  probe
gccaaccatttcttcactgtaccatttctgagagatgttgtcaatgtttatgaacctcag

SEQ ID NO: 147 - DNA, 60 bp, Artificial sequence
  probe
gctgagatgagaagaggaatgagccatatattggggaaaatcatagtttgtaggtataat

SEQ ID NO: 148 - DNA, 60 bp, Artificial sequence
  probe
cactttcacacttcatctcattcctgttgtcactttccccgaaacgaataaagtctcccc

SEQ ID NO: 149 - DNA, 60 bp, Artificial sequence
  probe
gaagtattattataattcaccataaacagctatctgtctgaattacttcaggccttctcc

SEQ ID NO: 150 - DNA, 60 bp, Artificial sequence
  probe
tatttgaacctttggaacttgggagttctcattgtaaccctaacatgtgagaataaaatg

SEQ ID NO: 151 - DNA, 60 bp, Artificial sequence
  probe
gcaaatcactgtaatgagaatggtactggaaaaatactgaatagacttgctaaatggcac

SEQ ID NO: 152 - DNA, 60 bp, Artificial sequence
  probe
tagcccattgccaaaaggttttactgtcttaaagctgtctttctgagatctaattccaag

SEQ ID NO: 153 - DNA, 60 bp, Artificial sequence
  probe
cttgtaaatagtatttaccagttagcaaagtctgtgttttcagaattacagtgagcacag

SEQ ID NO: 154 - DNA, 60 bp, Artificial sequence
  probe
gtgacgaccagccaggatatgctctcaatcatggagaaattggaattcttcgatttttct

SEQ ID NO: 155 - DNA, 60 bp, Artificial sequence
  probe
aagaatgggttctggagttctcatggtctttaggaaatattgagtaatttgtaatcaccg

SEQ ID NO: 156 - DNA, 60 bp, Artificial sequence
  probe
ttctgtaattatgaagaatcaaacaccaacaaagaagtatgatggctacacatcatgtcc

SEQ ID NO: 157 - DNA, 60 bp, Artificial sequence
  probe
acagaacgcaggttttggaatggtcttaaaagatgtgagggtgttaatctaggaaacttc

SEQ ID NO: 158 - DNA, 60 bp, Artificial sequence
  probe
tccagacgcactgatctttgcaaaggagacttaatttcaaatctgtaattaccatacata

SEQ ID NO: 159 - DNA, 60 bp, Artificial sequence
  probe
aagtatgcggttgaaattgattgcaaataatacaacagtggaacggaggtttagctcatg

SEQ ID NO: 160 - DNA, 60 bp, Artificial sequence
  probe
ttgtcggcttttatgtaatctgtaatatgtatagcaggaaatacgaagagttacacagtg

SEQ ID NO: 161 - DNA, 60 bp, Artificial sequence
  probe
atagacttaactcccttaagcccagacatctgttgagacctgacccctagtcattggtta

SEQ ID NO: 162 - DNA, 60 bp, Artificial sequence
  probe
atttgtcaacaaaggagaaaaagggatcatggttttggcaggagacacactgcccattga

SEQ ID NO: 163 - DNA, 60 bp, Artificial sequence
  probe
cttgcttagtctcctttcagtatttggcaataaaagaaagaagaaatagaacagctgaag

SEQ ID NO: 164 - DNA, 60 bp, Artificial sequence
  probe
gtaaggaacttgggtgttaatagttgagagctgtttagtaataacccagttttcttgagg

SEQ ID NO: 165 - DNA, 60 bp, Artificial sequence
  probe
gcaggagtagaaagatgctcatagcacacgcgatatgtatgataacatattttgtttcat

SEQ ID NO: 166 - DNA, 60 bp, Artificial sequence
  probe
ttcagaaagatcaagccaaagaactccaaacaagcagaaagggaagagaagcgagtcctc

SEQ ID NO: 167 - DNA, 60 bp, Artificial sequence
  probe
atgcataataacccagtttgtatcaaagggtatcgacttaagtgaaatttcaacatgctg

SEQ ID NO: 168 - DNA, 60 bp, Artificial sequence
  probe
gtgaagcccctttttcttgctaaaaccggcaattctccggttagaaatgttacttggtgt

SEQ ID NO: 169 - DNA, 60 bp, Artificial sequence
  probe
gcagggactgaatgacctgatgtcagatttagattcttcctggggattacacagctatga

SEQ ID NO: 170 - DNA, 60 bp, Artificial sequence
  probe
cgctggctcacacaaaacagctgacactgactaaggaactgcagcatttgcacaggggag

SEQ ID NO: 171 - DNA, 60 bp, Artificial sequence
  probe
aagtgcaaatgcctctttgaagcaattcaggctaggtaaaccgattttgccatttcaaaa

SEQ ID NO: 172 - DNA, 60 bp, Artificial sequence
  probe
tcatacatggtatacagatagctcataatgaagtccagaatcttacttttaagtgaaggc

SEQ ID NO: 173 - DNA, 60 bp, Artificial sequence
  probe
acacagtagcaaaagagaagatctcatttacaaatatctatggtgtttccttgttctgtg

SEQ ID NO: 174 - DNA, 60 bp, Artificial sequence
  probe
cttcaggactagaaagaagtcacaaatcttcaaaaggtggtagtagtagagatacaaaag

SEQ ID NO: 175 - DNA, 60 bp, Artificial sequence
  probe
cccccattctggaaggttttgttatcttcggaagaaccccaattatgatctctaagtgac

SEQ ID NO: 176 - DNA, 60 bp, Artificial sequence
  probe
tttgatgactgtacaacaggaagacttgaaaaatcacgtggattcatattaccaccgctc

SEQ ID NO: 177 - DNA, 60 bp, Artificial sequence
  probe
agagtcaggaataccatgtaacatgttgaagtagactaaagattagttctttggcaatag

SEQ ID NO: 178 - DNA, 60 bp, Artificial sequence
  probe
tcatattgagtaaatgaatgaaattgtgatttcctgagaatcgaaccttggttccctaac

SEQ ID NO: 179 - DNA, 60 bp, Artificial sequence
  probe
aaatactttacagtggtcggtcacaagaaaccatctgaacaatttcagtcatttgaagct

SEQ ID NO: 180 - DNA, 60 bp, Artificial sequence
  probe
taggggttgtaaagctactttattagatatagaatggcagattctctgatttaaaagggc

SEQ ID NO: 181 - DNA, 60 bp, Artificial sequence
  probe
ctggtagccaccggcactcacgaaccgaaaaaccaaagctatatggtaagaggctgtgca

SEQ ID NO: 182 - DNA, 60 bp, Artificial sequence
  probe
ctttgattagcagcggaacaaggagtcagacattttaagatggtggcagtagaggctatg

SEQ ID NO: 183 - DNA, 60 bp, Artificial sequence
  probe
ggaaagcttgtctgggttctatactgtgatctcatttgcctcgactacgatggaaacatt

SEQ ID NO: 184 - DNA, 60 bp, Artificial sequence
  probe
acagcattgagaaagagcatgtcgaagagatcagtgaacttttctatgatgccaagtcct

SEQ ID NO: 185 - DNA, 60 bp, Artificial sequence
  probe
cgagaatcaggagggactcaggaagatcacatcctacttcctcaatgaagggtcccaagc

SEQ ID NO: 186 - DNA, 60 bp, Artificial sequence
  probe
atttatcatttatctgaaatcacatgtagcagattgcatagtctgtaatcctctcagagg

SEQ ID NO: 187 - DNA, 60 bp, Artificial sequence
  probe
aggtggtgatctgaatacagcagcagtttgaaagtgttccgtttttaaataaacagtatg

SEQ ID NO: 188 - DNA, 60 bp, Artificial sequence
  probe
gaggatgagagcccagaggtaacagaacagcttcaggttatcgaaataacaatgttaagg

SEQ ID NO: 189 - DNA, 60 bp, Artificial sequence
  probe
caagccatctatccaggcattaattcatggactaaacagacattattactccattactat

SEQ ID NO: 190 - DNA, 60 bp, Artificial sequence
  probe
agaaaggtaacaatcttcattctacagatgaactcattgaaacaatttaggggaatgagg

SEQ ID NO: 191 - DNA, 60 bp, Artificial sequence
  probe
agatgggacagtaaatgttcagcattcttggatcagaagaaaacggactaattagatgct

SEQ ID NO: 192 - DNA, 60 bp, Artificial sequence
  probe
agatccagaacatgggaagttagggaaaatgtgtgattttgtgttttgaattactgtcag

SEQ ID NO: 193 - DNA, 60 bp, Artificial sequence
  probe
cagtgactacgagtagttctttctctattgaattattaggtccagaatagaagatgtcat

SEQ ID NO: 194 - DNA, 60 bp, Artificial sequence
  probe
gcaaaagagtattaatccactatctctagtgcttgactttaaatcagtacagtacctgta

SEQ ID NO: 195 - DNA, 60 bp, Artificial sequence
  probe
cgataagcctcatctgatggaagagaggaataaataattcacctatatgtgtttgaggtt

SEQ ID NO: 196 - DNA, 60 bp, Artificial sequence
  probe
cagggacagaggactgggtagcaggttccttctgttgtcctgtgtggtgtgtctactgtg

SEQ ID NO: 197 - DNA, 60 bp, Artificial sequence
  probe
cagagatgcgacctactcaatctgacttagtaaaaccatgctgtagaatttttgtcttaa

SEQ ID NO: 198 - DNA, 60 bp, Artificial sequence
  probe
tatgacttaaaccaactacaacttccctatagcttctaagcagtttcatcagcattactt

SEQ ID NO: 199 - DNA, 60 bp, Artificial sequence
  probe
ctgccaatgttgtgttttctgcttcatacgatattgcacagtactggtcagtatcaggaa

SEQ ID NO: 200 - DNA, 60 bp, Artificial sequence
  probe
gtcacagtgctaagttatctagttggctactattacaccttaaaaattgagtttacacac

SEQ ID NO: 201 - DNA, 60 bp, Artificial sequence
  probe
gacgacagctactggtttccactcctgcttcagaagaagaaattccacgggtacttcaag

SEQ ID NO: 202 - DNA, 60 bp, Artificial sequence
  probe
aagggccaaatgattcaaaacatcacaggtatttatgtgttttacaaagacctacattcc

SEQ ID NO: 203 - DNA, 60 bp, Artificial sequence
  probe
aaaccaatgattgtagcaaactcatactggatcatttcagttaccttgaactaatagcac

SEQ ID NO: 204 - DNA, 60 bp, Artificial sequence
  probe
ggagggaattgtattgatactccaatacctaaatttcacaataccacttaggaaagctat

SEQ ID NO: 205 - DNA, 60 bp, Artificial sequence
  probe
cccatcatcgatcttcatgaatgcaatatttatgatgtgaaaaatgacacaggattccag

SEQ ID NO: 206 - DNA, 60 bp, Artificial sequence
  probe
attacactttcaaagagaattccctttgcaattttatgtttggatcaccactgtaagcac

SEQ ID NO: 207 - DNA, 60 bp, Artificial sequence
  probe
aataagctttctcagaatgttggcaaatcacttcaatcctcaaatcagtcttccttgtgg

SEQ ID NO: 208 - DNA, 60 bp, Artificial sequence
  probe
agaacaggcttttagaagataaaagcgacaagaaggaatctggtgaattttagtcatccc

SEQ ID NO: 209 - DNA, 60 bp, Artificial sequence
  probe
aaatgatggccaaagtattagaaagaaacgaagagccactggagatggatcttctcctga

SEQ ID NO: 210 - DNA, 60 bp, Artificial sequence
  probe
gacaaagtgaagctgatcaaggaaatcaagaactacatccaaggcatcaacctcgtccag

SEQ ID NO: 211 - DNA, 60 bp, Artificial sequence
  probe
ccacttcctttcttgtgcttcgtgtcctgttgacggttacatttgtgtataattattata

SEQ ID NO: 212 - DNA, 60 bp, Artificial sequence
  probe
caaccttggccacttgagtttgtcctccaagggtaggtgtctcatttgttctggcccctt

SEQ ID NO: 213 - DNA, 60 bp, Artificial sequence
  probe
aagacgcagaaatgatgtcaagaatggcttctcatgggatcaaaattgtcattcagctaa

SEQ ID NO: 214 - DNA, 60 bp, Artificial sequence
  probe
aaggccacgtgaagatgctgcggctggtgtttgcacttgtgacagcagtatgctgtcttg

SEQ ID NO: 215 - DNA, 60 bp, Artificial sequence
  probe
ccctccagattactacggctatgaagattactatgatgattactatggttatgattatca

SEQ ID NO: 216 - DNA, 60 bp, Artificial sequence
  probe
cttttgcatgggttgatgagtggtatgacatgacaatggatgaagtccgagaatttgaac

SEQ ID NO: 217 - DNA, 60 bp, Artificial sequence
  probe
gggaaccttcatgactgttggaattgctctgtcataataagtcagggatatttagggggc

SEQ ID NO: 218 - DNA, 60 bp, Artificial sequence
  probe
atgtgaatgaagccatcaggctaatggagatgtcaaaggactctcttctaggagacaagg

SEQ ID NO: 219 - DNA, 60 bp, Artificial sequence
  probe
cctcccctggggcacagagatatattatatataaagtcttgaaaattttgaaatttggtg

SEQ ID NO: 220 - DNA, 60 bp, Artificial sequence
  probe
tgatgaatgctattaagaaatatcagaaaggcttctctgataaactggatttccttgagg

SEQ ID NO: 221 - DNA, 60 bp, Artificial sequence
  probe
agttgtagtgaattgctactgaaagctatcccaggtgatacagagctctttgtaaaccgc

SEQ ID NO: 222 - DNA, 60 bp, Artificial sequence
  probe
tacaagtttatttaaaactgctttctcaagtcgttattgatacagcaagtgaacctgctg

SEQ ID NO: 223 - DNA, 60 bp, Artificial sequence
  probe
tgataatatattctgcacggtaagaattccttttacagacattctttatcaagaggtcgg

SEQ ID NO: 224 - DNA, 60 bp, Artificial sequence
  probe
atctatgaccggaaattcctgatggagtgtcggaactcacctgtgaccaaaacaccccca

SEQ ID NO: 225 - DNA, 60 bp, Artificial sequence
  probe
ccatcaccaaaacgtgcaacaccatgaaccagtttgtgaacaagttcaatgtcctctacg

SEQ ID NO: 226 - DNA, 60 bp, Artificial sequence
  probe
cagtgattaacaatgccaaaaaatgcaagtaactagccattgttcaaatgacagtggtgc

SEQ ID NO: 227 - DNA, 60 bp, Artificial sequence
  probe
tttttatttgtctgtagacagggaacatgatgggcactgacctcctgtaaagaataaaac

SEQ ID NO: 228 - DNA, 60 bp, Artificial sequence
  probe
cccacagcaataaaagcttccccctgatatccatccctttgtagtttgaacaaatatatt

SEQ ID NO: 229 - DNA, 60 bp, Artificial sequence
  probe
agtgaagagtgagtgaaaggaagaattcagtgaatacattgataccttgatattatctgc

SEQ ID NO: 230 - DNA, 60 bp, Artificial sequence
  probe
gttcttgaggtagaaatgtctacagtcagttgtttcatctagcttgcatcttaaaacaca

SEQ ID NO: 231 - DNA, 60 bp, Artificial sequence
  probe
caggtgaatggattattctaaacagatcaccatccatcttcaagaagctactttgaaagt

SEQ ID NO: 232 - DNA, 60 bp, Artificial sequence
  probe
gtgcctcctttattaaggggttctttgagaataaaagagaaaagacctactttatttgac

SEQ ID NO: 233 - DNA, 60 bp, Artificial sequence
  probe
acccgcaaggtaacaaatcggaagtacccaaaactgaaagaagtggatgatgtgcttggt

SEQ ID NO: 234 - DNA, 60 bp, Artificial sequence
  probe
gcatcaagagtcaagttgctatatggtcaaaggttaaatttatctctaaaaaatggcagg

SEQ ID NO: 235 - DNA, 60 bp, Artificial sequence
  probe
tctgggaatagaaagtcaccatgattttttggtgaaagtaaaagttggaaaattcatggc

SEQ ID NO: 236 - DNA, 60 bp, Artificial sequence
  probe
ggagactaaacctggtgctcaggagcgaaggactgtgaacttgtagcctgaagagccaga

SEQ ID NO: 237 - DNA, 60 bp, Artificial sequence
  probe
ctgccctctcagcttgtgagacaacacaggagccttctatagtatgttgatatgctagat

SEQ ID NO: 238 - DNA, 60 bp, Artificial sequence
  probe
aaacttgtgtagggaatcctgacttttaaaatgtgagggtatttggatctgtgttgaaag

SEQ ID NO: 239 - DNA, 60 bp, Artificial sequence
  probe
attttttagaaaatacacacttttcaggagaaacctgagcatgattttggattctccacc

SEQ ID NO: 240 - DNA, 60 bp, Artificial sequence
  probe
gggagaacatctgatgttaacttgacagtcttgtctcgtgtattgaattcgtgccaaagg

SEQ ID NO: 241 - DNA, 60 bp, Artificial sequence
  probe
gagataacctagctctttatatcttcccttttaaatagaaacaactgtcttgagaagctc

SEQ ID NO: 242 - DNA, 60 bp, Artificial sequence
  probe
ggatcttgtcagattcactgaaaagaggtacaaatctattgtcaagtacaagacagcttt

SEQ ID NO: 243 - DNA, 60 bp, Artificial sequence
  probe
aaatgctgacaaaaatattttcctagcatcagtagatttctggcatatgtttctgctagc

SEQ ID NO: 244 - DNA, 60 bp, Artificial sequence
  probe
ttcaaagctggagtctgtcctcctaagaaatctgcccagtgccttagatacaagaaacct

SEQ ID NO: 245 - DNA, 60 bp, Artificial sequence
  probe
catctgcgcataaaggaccagatgaaactctgaggattaaagctgagtatgaaggggatg

SEQ ID NO: 246 - DNA, 60 bp, Artificial sequence
  probe
cacacacacacttagtcttgtaatttcaggccagaaattctcaacactattttgcatctg

SEQ ID NO: 247 - DNA, 60 bp, Artificial sequence
  probe
aagttatttatggcctgggaaacaatttgcatttgtccccaaatacgcttagctgtgtgc

SEQ ID NO: 248 - DNA, 60 bp, Artificial sequence
  probe
ctgaaagtgatactcttggatgtgattttgaatttcaagaaagcatccattctctatcac

SEQ ID NO: 249 - DNA, 60 bp, Artificial sequence
  probe
agccaaggaccgattccaggcactttctgtagcaaatgactgtgaattacgacttctctt

SEQ ID NO: 250 - DNA, 60 bp, Artificial sequence
  probe
tgggcctttctcagaagactgtaatgtacctgaagtttctgaaatattgcaaacccacag

SEQ ID NO: 251 - DNA, 60 bp, Artificial sequence
  probe
aaaggaaaaagtttcagacaagcaattacccagtttccttatctataaaatggggacatc

SEQ ID NO: 252 - DNA, 60 bp, Artificial sequence
  probe
ctcaccaaatggtcaaacaactaggtatggagaaaatgaaaaattaccagactacatcaa

SEQ ID NO: 253 - DNA, 60 bp, Artificial sequence
  probe
actcgtgatcattgagaaagtgtttgaaactttctcatgaagtgtatatataatggcgtg

SEQ ID NO: 254 - DNA, 60 bp, Artificial sequence
  probe
ccgattgctcctgtgtaaagatgccttgtcgtgcagaaacaaatggctgtccagtttatt

SEQ ID NO: 255 - DNA, 60 bp, Artificial sequence
  probe
cttgtcctgaggtgttgagggtgtcacgagcccattctcatgtttacaggggttgtgggg

SEQ ID NO: 256 - DNA, 60 bp, Artificial sequence
  probe
gttatctcatgagagctgtgatcctttaaagaaacctaaacatttcatagacttttgcac

SEQ ID NO: 257 - DNA, 60 bp, Artificial sequence
  probe
tatcaagataccccaaaacagattcggagtaagatcaacgtctataaacgcttttaccca

SEQ ID NO: 258 - DNA, 60 bp, Artificial sequence
  probe
ttaaactgaccacttggaagaaacaccttggtatctgtggttttcttgccttgtccctgc

SEQ ID NO: 259 - DNA, 60 bp, Artificial sequence
  probe
ccagacaatgaaataaccatcaaaaagctaaggaaaaaggttttagctcagtactacaca

SEQ ID NO: 260 - DNA, 60 bp, Artificial sequence
  probe
caactttcccatcttgtctctcttggatgatgtttgccgtcagcattcaccaaaataaac

SEQ ID NO: 261 - DNA, 60 bp, Artificial sequence
  probe
tgacccaagtctttgcctctactcccttaacagtgttgaattcaactgaaggcgaggaat

SEQ ID NO: 262 - DNA, 60 bp, Artificial sequence
  probe
tttgagttcatgaaacagaatcaggatgccttcgacgtgatcatcactgactcctcagac

SEQ ID NO: 263 - DNA, 60 bp, Artificial sequence
  probe
ggcaacatctgtctagtgttgtcttcatcaagaacagactatatactaattcccactaga

SEQ ID NO: 264 - DNA, 60 bp, Artificial sequence
  probe
gctcctttgtaaaaagttaaagatttgaaagagaatctcatattcccgaggcattaggaa

SEQ ID NO: 265 - DNA, 60 bp, Artificial sequence
  probe
gggagcataaaacatccttgacaggtagagagagttttgaatggcttttgttatttaaaa

SEQ ID NO: 266 - DNA, 60 bp, Artificial sequence
  probe
agctctttgctactgctatggtaagatatcaaatgaaaaacctctgtaaaacatttccag

SEQ ID NO: 267 - DNA, 60 bp, Artificial sequence
  probe
tgaggaagattttgttagacttaaacagcagatttcagatcatatttcacaatcctgtgg

SEQ ID NO: 268 - DNA, 60 bp, Artificial sequence
  probe
gagccagttaattttaagaatttcacacatttagccaatctttctagatgtctctgaagg

SEQ ID NO: 269 - DNA, 60 bp, Artificial sequence
  probe
gaatacggtaaaataaatgactttaaccaagtagctataatgggacttagcactgtatgc

SEQ ID NO: 270 - DNA, 60 bp, Artificial sequence
  probe
agttggaaaaggataatacagactgcactaaatgttttcctctgttttacaaactgcttg

SEQ ID NO: 271 - DNA, 60 bp, Artificial sequence
  probe
gtgctcaggaatttgaaaacgctgctatacttactctggttactacatttcttccactcc

SEQ ID NO: 272 - DNA, 60 bp, Artificial sequence
  probe
ttacccatgataggacttttgtgatatggctaatctcagtacacatttcaacttaaaacc

SEQ ID NO: 273 - DNA, 60 bp, Artificial sequence
  probe
taattccttccctatctccttaccaaagtacaagtcacatctttcccaccttttctgcaa

SEQ ID NO: 274 - DNA, 60 bp, Artificial sequence
  probe
ttctgccttttgctggtgtttctggaatttgctttccctcacctctcacttccttctaga

SEQ ID NO: 275 - DNA, 60 bp, Artificial sequence
  probe
ttcgcaaaccaggacgactacatatcatttcggcaccatgtgtataagaagacagaccac

SEQ ID NO: 276 - DNA, 60 bp, Artificial sequence
  probe
ccctgcttaaatggtggaaaatgtgtaagaccaaaccgatgtcactgtctttcttcttgg

SEQ ID NO: 277 - DNA, 60 bp, Artificial sequence
  probe
ttgctttgtctccctggatatggatttcagttaagtattttgtaacccgttacactgtgt

SEQ ID NO: 278 - DNA, 60 bp, Artificial sequence
  probe
ttgagctgcattcaggaagtgcgggacatggtaggggaggcaaaaagccttgggcactac

SEQ ID NO: 279 - DNA, 60 bp, Artificial sequence
  probe
catgcattggatgttttgctaaataactcctgtggatttaggaatgtgtgctaatagcaa

SEQ ID NO: 280 - DNA, 60 bp, Artificial sequence
  probe
gctttaagatcacgcagaacaacgccatgaagatctcctttgccaagaagtagcaccttt

SEQ ID NO: 281 - DNA, 60 bp, Artificial sequence
  probe
acgatcctcctccaagacaaacagcggaaaatctactgcgtggcttgtcaggaactcgac

SEQ ID NO: 282 - DNA, 60 bp, Artificial sequence
  probe
caccttcgcaccgtgcccaggtacactttcaagacactgtaaccacaagatgttatttat

SEQ ID NO: 283 - DNA, 60 bp, Artificial sequence
  probe
caaatgattccggatctctaaaaggctctctcagatgaaaagggagtaaaggaaaaaaga

SEQ ID NO: 284 - DNA, 60 bp, Artificial sequence
  probe
gcagctgtagatacaaaaagtcctctaatggagtactggaatgtttcattgtcttcacaa

SEQ ID NO: 285 - DNA, 60 bp, Artificial sequence
  probe
atattttgctcactatccctccccacttgcttccctgatatgtgctctgacttccctgaa

SEQ ID NO: 286 - DNA, 60 bp, Artificial sequence
  probe
gttcctattaaagaacatcaacgatgcttgggtttgcaccaatgacatgttcaggctcgc

SEQ ID NO: 287 - DNA, 60 bp, Artificial sequence
  probe
catttggagacagagccatttggatattttccccttgaacttctccatgacctgaagcgt

SEQ ID NO: 288 - DNA, 60 bp, Artificial sequence
  probe
gtgttacctcaatttcagagtaagtagtggttgattagtaatgtagtatacactggcaga

SEQ ID NO: 289 - DNA, 60 bp, Artificial sequence
  probe
aatgacaggaagatgttcttgtttggaacctacctgactaagaatggctcagagataccc

SEQ ID NO: 290 - DNA, 60 bp, Artificial sequence
  probe
gttgggctatcaatggaagaaaagttgtatgaatcaggttactatatcaacaactgatag

SEQ ID NO: 291 - DNA, 60 bp, Artificial sequence
  probe
tgtgcccgatctacttgtcattgcagacaaatatgatcctttcactacgacaaataccga

SEQ ID NO: 292 - DNA, 60 bp, Artificial sequence
  probe
tgtggtgtcactaaaagaaggtatgttcaacaatgcaaacgttcttttcaaaaccaagtt

SEQ ID NO: 293 - DNA, 60 bp, Artificial sequence
  probe
agccaagggtctttcacatcacctatccctacatacataccaaatggaaaagtggccatc

SEQ ID NO: 294 - DNA, 60 bp, Artificial sequence
  probe
cttgtaatccaaagttactttgtacagaccttgaagattgaaaaaacacgtctaaagagc

SEQ ID NO: 295 - DNA, 60 bp, Artificial sequence
  probe
agagggaagacattaaggggattggggacatttgtttcacacatctgcagtaatatgagt

SEQ ID NO: 296 - DNA, 60 bp, Artificial sequence
  probe
ccttgaaaatcagattctaactgattgtatgcaactaagtatttctgaacacctatgcag

SEQ ID NO: 297 - DNA, 60 bp, Artificial sequence
  probe
cttgtctctcttggatgatgtttgccgtcagcattcaccaaaataaacttgctctctggg

SEQ ID NO: 298 - DNA, 60 bp, Artificial sequence
  probe
ctcactgtcctgttttcttaggcactgagggatctgagtaggtctgggatggacaggaaa

SEQ ID NO: 299 - DNA, 60 bp, Artificial sequence
  probe
gtacaggatggattatggagaatttggtaatctagtcggaataattatattgtattgggc

SEQ ID NO: 300 - DNA, 60 bp, Artificial sequence
  probe
ctaagtcatactactgatggaatgagaatacaaaccaaaaacactttattcaagtccagg

SEQ ID NO: 301 - DNA, 60 bp, Artificial sequence
  probe
ttccttactttgcacagtgaacacaactaaccacattaattcagctttgtgaagtccctg

SEQ ID NO: 302 - DNA, 60 bp, Artificial sequence
  probe
gattcctacctctcatgattactatggagattgaataattggtaaaattctcctagctca

SEQ ID NO: 303 - DNA, 60 bp, Artificial sequence
  probe
tcatcttcattgatgagttggatgccatcggcaccaagcgctttgacagtgagaaggctg

SEQ ID NO: 304 - DNA, 60 bp, Artificial sequence
  probe
atggaggatggattggaaggcatgatgttctgacttctgtcagttattcttgcaaagatt

SEQ ID NO: 305 - DNA, 60 bp, Artificial sequence
  probe
caagcctaccacaatcatcgaaaagaatggggacattctcaccctaaaaacacacagcac

SEQ ID NO: 306 - DNA, 60 bp, Artificial sequence
  probe
catctgcttagcttagttctacaaactttttcatttttaatgtgcaaagaacaggcgggg

SEQ ID NO: 307 - DNA, 60 bp, Artificial sequence
  probe
caattgtcagagtacaacgtgatggcaacaaactcatagagtttaataatggccaaagag

SEQ ID NO: 308 - DNA, 60 bp, Artificial sequence
  probe
cctaattttctctcctggtagctgaacaaaggtctaaattagcttaacaaaagaacaggc

SEQ ID NO: 309 - DNA, 60 bp, Artificial sequence
  probe
gacaaactctcttactccttaagacaaatgctcacctgatcaatatggggaaataagctg

SEQ ID NO: 310 - DNA, 60 bp, Artificial sequence
  probe
ggcataaaacaggttcagaagaatcgacaatgatgtcatatttttccaagtaccaaattc

SEQ ID NO: 311 - DNA, 60 bp, Artificial sequence
  probe
tattgaaccatggcttaatcaaatcatgcctaagaaagatcctgtcaaaatagtccgatg

SEQ ID NO: 312 - DNA, 60 bp, Artificial sequence
  probe
ggaaagcaaaagacgatgccacattgtcaggaaaacgaatgcagtcactctccctgaata

SEQ ID NO: 313 - DNA, 60 bp, Artificial sequence
  probe
cagagttcccatacctcctagaccagtaaagccagattatagaagatggtcagcagaagt

SEQ ID NO: 314 - DNA, 60 bp, Artificial sequence
  probe
cctagctgtcttcttgaacttgggactctcctttcccaagacttccatcactagctcctg

SEQ ID NO: 315 - DNA, 60 bp, Artificial sequence
  probe
ctccttgaaagctttgcctttgttttgaacttcctttcccacttggtagaaagagcccag

SEQ ID NO: 316 - DNA, 60 bp, Artificial sequence
  probe
tgaagggctttctcaccccagctctggctatgcccagttctctgagaaaggagctcagtg

SEQ ID NO: 317 - DNA, 60 bp, Artificial sequence
  probe
actgtggaaagccatgagtccagcctcattctcccacccatcaaggtcatggtggccttg

SEQ ID NO: 318 - DNA, 60 bp, Artificial sequence
  probe
accagacctaatttgcaagtattgggtcttcaaacttcaagtgcaatgtattatgaaaac

SEQ ID NO: 319 - DNA, 60 bp, Artificial sequence
  probe
cattcatgccaagtatctttccagcatgtttctcccatttagaatatctagcatgtaagg

SEQ ID NO: 320 - DNA, 60 bp, Artificial sequence
  probe
tccagttgaagattactggatgcacgtacagaatacattcagccgtcaggtaataacatg

SEQ ID NO: 321 - DNA, 60 bp, Artificial sequence
  probe
ctaggttgaagtctagaatgaaagaaatctgaatccatgtcattcataaccccttgatct

SEQ ID NO: 322 - DNA, 60 bp, Artificial sequence
  probe
acaagaacaggtttatcgtgtccaaaaactccaccatattctagaaatattagtcagttg

SEQ ID NO: 323 - DNA, 60 bp, Artificial sequence
  probe
ctcacattgtacaatattgcaaaaattcatgcttgagagaaacaaaaacactgagagtgg

SEQ ID NO: 324 - DNA, 60 bp, Artificial sequence
  probe
aacacctgtttttattcccctgagatctaaacctgtttatggaaaaagatggtctagttc

SEQ ID NO: 325 - DNA, 60 bp, Artificial sequence
  probe
tcttttccccaacctaaaaccaaccaccagcatttcactacaggaccaaatggaaaccga

SEQ ID NO: 326 - DNA, 60 bp, Artificial sequence
  probe
gatatgagctcctcatagaaaccagacctactgtattagacagtaacctctaacctcacc

SEQ ID NO: 327 - DNA, 60 bp, Artificial sequence
  probe
tacaaccccagagttttaaggacttggaaaggaagcacaatcagagaagaaaacagcccc

SEQ ID NO: 328 - DNA, 60 bp, Artificial sequence
  probe
gttgcccttaaaccatttctagctgttaaccctatccagaaaaatgattgagtgatagct

SEQ ID NO: 329 - DNA, 60 bp, Artificial sequence
  probe
cagcataaactcagagatgaagacctctttcgtttaaaagagcctgtactgtttgtgtca

SEQ ID NO: 330 - DNA, 60 bp, Artificial sequence
  probe
tggtatagcaagaaggtattctgagcattatatcaatggggaagttatagatcttttgca

SEQ ID NO: 331 - DNA, 60 bp, Artificial sequence
  probe
cttcctagcaatagtctgcatttaaagaaaggtgtgttcaattcatcagcttgaaattga

SEQ ID NO: 332 - DNA, 60 bp, Artificial sequence
  probe
gagtttgtcttctacgaccagctgaagcaagtgatgaatgcgtacagagtcaagccggcc

SEQ ID NO: 333 - DNA, 60 bp, Artificial sequence
  probe
tcttttctatctgatccacatggagaggttaaaggttcaatttcatgacctctatgcagg

SEQ ID NO: 334 - DNA, 60 bp, Artificial sequence
  probe
tatcagggttttgggtgtcacttaggttttgtccatcagattctgtgagacaccaggcat

SEQ ID NO: 335 - DNA, 60 bp, Artificial sequence
  probe
ctggaatacattaaagaagcaaaagaacctcatacaaaagtcattgagtaaatgacagcc

SEQ ID NO: 336 - DNA, 60 bp, Artificial sequence
  probe
agaaagaagcctttttcattaaggatacaacctatttgtagctcgcactttaaaagatgc

SEQ ID NO: 337 - DNA, 60 bp, Artificial sequence
  probe
tatgtttcttccaaggaaacaggagagactgaattaataattctctctttcctcttaagc

SEQ ID NO: 338 - DNA, 60 bp, Artificial sequence
  probe
acacacaggatctctccgagcagcaattccccattcctctcccctattgctggctctgca

SEQ ID NO: 339 - DNA, 60 bp, Artificial sequence
  probe
aaggattttggaaactcaaggaaaagttctttcaacctatacaacctaacttatggactg

SEQ ID NO: 340 - DNA, 60 bp, Artificial sequence
  probe
ctgtggtgaagtgttcctggtgttccctttccctgctgaccttggaggaatgacaataaa

SEQ ID NO: 341 - DNA, 60 bp, Artificial sequence
  probe
gttttatccatcaaaaaatgcactctcacagtggagttgaagtctaatacatgatacatg

SEQ ID NO: 342 - DNA, 60 bp, Artificial sequence
  probe
cttattgagtactctgtttctacgtatgtagaatgtatagggatagaagagttgaaaagg

SEQ ID NO: 343 - DNA, 60 bp, Artificial sequence
  probe
aacatgatcaagtccttctatactgcaagtcttttgatagatgtcataacagtattggag

SEQ ID NO: 344 - DNA, 60 bp, Artificial sequence
  probe
aaactaaaataaggaatagaaaagctgtttttcaggctgacagtccaattaagggtagcc

SEQ ID NO: 345 - DNA, 60 bp, Artificial sequence
  probe
gacatgctaccctaataatgaaataggtatcctggacaaaacaataaaaaggaagattgc

SEQ ID NO: 346 - DNA, 60 bp, Artificial sequence
  probe
tgcaagagaaggtgccaaagtcatagccacagacattaatgagtccaaacttcaggaact

SEQ ID NO: 347 - DNA, 120 bp, Artificial sequence
  probe
cattgtttctcaaagctccttgattaagagaaagaacagaaatttgcacagaagatagtggcaagctcagtcatacatgacaaagtgtaattaacactgatgtttgtgttaaatttgcag

SEQ ID NO: 348 - DNA, 60 bp, Artificial sequence
  probe
gcaagctcagtcatacatgacaaagtgtaattaacactgatgtttgtgttaaatttgcag

SEQ ID NO: 349 - DNA, 60 bp, Artificial sequence
  probe
taatgtaaaactggttgaacaaaaccgatgcctgattgaatgaaaacaattctatgaggc

SEQ ID NO: 350 - DNA, 60 bp, Artificial sequence
  probe
actagtataaaactgtaaatactactataagacattggctttttccagacatggatccgg

SEQ ID NO: 351 - DNA, 60 bp, Artificial sequence
  probe
caaaacgtagcttagcaaagctgttactaaactggagagatcctttaatacatagaaatg

SEQ ID NO: 352 - DNA, 60 bp, Artificial sequence
  probe
cagggtattcttccaggagatttgaccagcaagtggaaaaataccataaaccctgctgaa

SEQ ID NO: 353 - DNA, 60 bp, Artificial sequence
  probe
ggcttatagagttagaagtcagtatttcttctaatctgaggctatgatcagtcccagctg

SEQ ID NO: 354 - DNA, 60 bp, Artificial sequence
  probe
agatactgtatctggctgtactatactaacagtgatttgcctgcatgtgtttgatagaga

SEQ ID NO: 355 - DNA, 60 bp, Artificial sequence
  probe
ttgaatctcttgccaaaagacatgggtaaaatatctgcctctctcatagagattttaaga

SEQ ID NO: 356 - DNA, 60 bp, Artificial sequence
  probe
gtgaagacaagaataaagccactagtagaagcatcggattagacacaacctcgtcacaac

SEQ ID NO: 357 - DNA, 60 bp, Artificial sequence
  probe
tgtcagcagacatgttcatccgatgatagtactgcagttttctattaataatttgcagac

SEQ ID NO: 358 - DNA, 60 bp, Artificial sequence
  probe
ctagatcgaagcaaaaccgattttgctttcaccattaaggttgcattttaatgcagttat

SEQ ID NO: 359 - DNA, 60 bp, Artificial sequence
  probe
gatgccaaaattagtcttctcaaagctttgagtagagtaagtgtgggaataagccagttt

SEQ ID NO: 360 - DNA, 60 bp, Artificial sequence
  probe
gtcatcaagcacaaccggttcacaaaggagacctatgacttcgacatcgccgtgctccgg

SEQ ID NO: 361 - DNA, 60 bp, Artificial sequence
  probe
gaaatatcagacacactctgctcattcatgtctccttccacagtttatttcctcgcttcc

SEQ ID NO: 362 - DNA, 60 bp, Artificial sequence
  probe
taaaataacagcaagtttatgtgatcttaaatcaaggcttgattcaagaactggtgtggc

SEQ ID NO: 363 - DNA, 60 bp, Artificial sequence
  probe
gaatgctgtttgtgaggtgtctacagggtttatagtagtcttgtggacacagaaatgcac

SEQ ID NO: 364 - DNA, 60 bp, Artificial sequence
  probe
caggtggtgactatcctgttgagaagcaaaagatactttgcaagtaaaaaatatgcctcc

SEQ ID NO: 365 - DNA, 60 bp, Artificial sequence
  probe
cttaaagttacggggcttcgttgaatgctccttcccctaggcagctggtaatagaggggt

SEQ ID NO: 366 - DNA, 60 bp, Artificial sequence
  probe
gccctgttctataaatatctataaatactcatatatatacacacctacacatggccaacc

SEQ ID NO: 367 - DNA, 60 bp, Artificial sequence
  probe
ggtgatgttcaacagaagtgaagacaaaacaatatgcatcaggagaacaagagtaaaccc

SEQ ID NO: 368 - DNA, 60 bp, Artificial sequence
  probe
ggtttcaagttcttacccttctccttcgagcatattcgggaagggaagtttgaagagtga

SEQ ID NO: 369 - DNA, 60 bp, Artificial sequence
  probe
agcaataagctggactagtgtcctaaaaatggctaactgatgaattagaagccatctgac

SEQ ID NO: 370 - DNA, 60 bp, Artificial sequence
  probe
ctgatgtggtcctagtctccgccgggtttgatgctgttgaaggacatctgtctcctctgg

SEQ ID NO: 371 - DNA, 60 bp, Artificial sequence
  probe
tgcaaaaaactacagacatctctgtgctgtttattacaacaagaatcctgggtttgagat

SEQ ID NO: 372 - DNA, 60 bp, Artificial sequence
  probe
tccagtgaaagtgtgaacctgcggatcgctgccggtgaaaccattgcactgctctttgag

SEQ ID NO: 373 - DNA, 60 bp, Artificial sequence
  probe
acgttttctgagaaaaaatagcatgaagcctaaagccatttcttccaaaaacaacattgc

SEQ ID NO: 374 - DNA, 60 bp, Artificial sequence
  probe
ccacaacaagtacatggtgactgaacgcgccacctacatcggaacctccaactggtctgg

SEQ ID NO: 375 - DNA, 60 bp, Artificial sequence
  probe
agaaagccagaaattccatcaatacatctagacagatgtttgcttgtagtttttggtatc

SEQ ID NO: 376 - DNA, 60 bp, Artificial sequence
  probe
tagactacctcagaatgtagtcaagaattcacccagtgttctttggatgtcccttcccta

SEQ ID NO: 377 - DNA, 60 bp, Artificial sequence
  probe
catgaactctacaagtacatcaacaagtactatgaccagatcatcactgccctggaggag

SEQ ID NO: 378 - DNA, 60 bp, Artificial sequence
  probe
ccatctgaaattggatagcccactgaaattgaacatgccttctcttataaatgtgtggtg

SEQ ID NO: 379 - DNA, 60 bp, Artificial sequence
  probe
cccatctccattgcattcatgtactaccctcagtctacactcacaatcatcttctcccaa

SEQ ID NO: 380 - DNA, 60 bp, Artificial sequence
  probe
tgtcacccagttcagtacttggtacataaaagacaataaatgtttaactgagccaagatt

SEQ ID NO: 381 - DNA, 60 bp, Artificial sequence
  probe
agaaataagaatcaagagcagctattgttatccttagagtgttttccgtctagggccggc

SEQ ID NO: 382 - DNA, 60 bp, Artificial sequence
  probe
aattgcaatatttgcaaacatgtgccacaacagtgttcttggtgatgtttctaaaacagt

SEQ ID NO: 383 - DNA, 60 bp, Artificial sequence
  probe
gtaaattcttcaggaataaacttcatgctaggttaacatcaatgtatggttacagcttcc

SEQ ID NO: 384 - DNA, 60 bp, Artificial sequence
  probe
gatttacctgaaaatcttcacaactgatcattatctccttctctttgagacctgactgaa

SEQ ID NO: 385 - DNA, 60 bp, Artificial sequence
  probe
caaaatgagtttcagccagagtttcagtttgacttaagtcaattaagcaacatctcaagg

SEQ ID NO: 386 - DNA, 60 bp, Artificial sequence
  probe
ttacattataaactcttttgaaaaatgtatctaaaattttttaagttctgttttgattcc

SEQ ID NO: 387 - DNA, 60 bp, Artificial sequence
  probe
cagtttcctctcatcaaatctaaaattctccaaaatactctcaggcataacatacttagc

SEQ ID NO: 388 - DNA, 60 bp, Artificial sequence
  probe
tccttgattgtgctatgtttcagtggaagaaattctttgaagtagatgtgagtgaaaaac

SEQ ID NO: 389 - DNA, 60 bp, Artificial sequence
  probe
tgagatcctgcaatgagcaatcctttaagcactttcttccccgttatgctccaaagtacg

SEQ ID NO: 390 - DNA, 60 bp, Artificial sequence
  probe
aaatgaccactagaataaagaatttttccaaacatcagtgtcaagctcttccaatcaacc

SEQ ID NO: 391 - DNA, 60 bp, Artificial sequence
  probe
tttcatgaaactgaataaaagaaatcctttccgagatagaggagatttgtaggtgaatgc

SEQ ID NO: 392 - DNA, 60 bp, Artificial sequence
  probe
ccaaaagtcaagaattcaaccagcaattaaataataattcctgtggtaaagcactttccc

SEQ ID NO: 393 - DNA, 60 bp, Artificial sequence
  probe
tctgtacttcctagacaacctccattattccctaagggaatcagtgttgtgtctgtctac

SEQ ID NO: 394 - DNA, 60 bp, Artificial sequence
  probe
aaacaaaggcacgaccagataagtgacttcaagggagtaaaggtcgttctatcggagaac

SEQ ID NO: 395 - DNA, 60 bp, Artificial sequence
  probe
ggctcagagaatcctgcaaaatccttttttattgtttattctgcagacaaaaatgtgaca

SEQ ID NO: 396 - DNA, 60 bp, Artificial sequence
  probe
tgtgaaaagaaaacaaacaacttgtaccttctttctgatatcaccgtcattcctctttct

SEQ ID NO: 397 - DNA, 60 bp, Artificial sequence
  probe
cccagggactttaaattgaagtcctagggttactcagtatagggaggaaatgaggaccaa

SEQ ID NO: 398 - DNA, 60 bp, Artificial sequence
  probe
ttctccttcaaaacactcatctcctctgtaatgatcagatgaactcatgtctttctcaga

SEQ ID NO: 399 - DNA, 60 bp, Artificial sequence
  probe
catgcacattgtcatgtgcagctttgcatatacacacatgcatacatgagcctccacaca

SEQ ID NO: 400 - DNA, 60 bp, Artificial sequence
  probe
atggacaaatagctgtgtaaatagcttctcataaaacgcttctactgatagctgtttgac

SEQ ID NO: 401 - DNA, 60 bp, Artificial sequence
  probe
gtgctcattaattcacgaggcaggataaaatagaaatttcaaattctgattgaataggcg

SEQ ID NO: 402 - DNA, 60 bp, Artificial sequence
  probe
ggaagctgaaatggtaccataaatgctgctaatctgagagccattttaaactgcactgct

SEQ ID NO: 403 - DNA, 60 bp, Artificial sequence
  probe
ccctccataatgaaagaagataggtttatgcttctctttccattaatgtgcagccattct

SEQ ID NO: 404 - DNA, 60 bp, Artificial sequence
  probe
ttaattgaaataatgcttaaaggctgttgcttctctttaaaattgacccaaggcatgagc

SEQ ID NO: 405 - DNA, 60 bp, Artificial sequence
  probe
acttcgaaggtatgtttacaggatggattagcatgcactttacagatatttatgaagttg

SEQ ID NO: 406 - DNA, 60 bp, Artificial sequence
  probe
ctccaaaaaatttagccaaaggtcgtgccccaaaatttacgcaaccactacaacctcgca

SEQ ID NO: 407 - DNA, 60 bp, Artificial sequence
  probe
agtttcaaatttacctcagcgtttgtgtatcgggcaaaaatcgttttgcccgattccgta

SEQ ID NO: 408 - DNA, 60 bp, Artificial sequence
  probe
tgaacgtaaaatgtgtcgctccgtatccttctctgttgtcatcagaagataacgctgatg

SEQ ID NO: 409 - DNA, 60 bp, Artificial sequence
  probe
gagaataaagtgtgcaatcgctatgagtttctgaatggaagagtccaatccaccgaactc

SEQ ID NO: 410 - DNA, 60 bp, Artificial sequence
  probe
gcttaagaacatcaacagcatcaactttgacaaccccgtctatcagaagaccacagagga

SEQ ID NO: 411 - DNA, 60 bp, Artificial sequence
  probe
gccagaccatctctaccctgtacttggacaacttaacttttttaaccaaagtgcagttta

SEQ ID NO: 412 - DNA, 60 bp, Artificial sequence
  probe
acacaaaaggaggcaaataagaattttgaaaaatctctgctcaaagaattcaagcgtctg

SEQ ID NO: 413 - DNA, 60 bp, Artificial sequence
  probe
gtactttcttcccttaaatcatggtattcttctgacagagccatatgtaccctaccctgc

SEQ ID NO: 414 - DNA, 60 bp, Artificial sequence
  probe
gcaagattttagtaaagttgtacctcccctggatgaagatggacggagcttgttatcgca

SEQ ID NO: 415 - DNA, 60 bp, Artificial sequence
  probe
taaaatggatcctgcaccacgggaacctcacagcacctcacttcttttggttttctttct

SEQ ID NO: 416 - DNA, 60 bp, Artificial sequence
  probe
ttcggatgggctgtttagatgttatataatccacaaaaggttcattgagctaaaaaagtg

SEQ ID NO: 417 - DNA, 60 bp, Artificial sequence
  probe
ggccaacagttaaatgagaacatgaaaacagaaaacggttaaaactgtccctttctgtgt

SEQ ID NO: 418 - DNA, 60 bp, Artificial sequence
  probe
tgcatgaactctacaagtacatcaacaagtactatgaccagatcatcactgccctggagg

SEQ ID NO: 419 - DNA, 60 bp, Artificial sequence
  probe
caaagtagatcgttattttgatcaaactgtgcaaacagtagtaccacgtgtagcattttg

SEQ ID NO: 420 - DNA, 60 bp, Artificial sequence
  probe
caaagtatttttcaatcagagttttcagaacctgacattgttaaagatactgcttgtccc

SEQ ID NO: 421 - DNA, 60 bp, Artificial sequence
  probe
gtgaagcaagtgcaggaagaagaccccaacactcacatcctctacgcctgtgactcctgt

SEQ ID NO: 422 - DNA, 60 bp, Artificial sequence
  probe
tagccaaagccataaaaaacctgcaacgtagagaaaataatgcagataccctgactagcc

SEQ ID NO: 423 - DNA, 60 bp, Artificial sequence
  probe
tgttcacgtgttttccatcccacataaatacagtattcccactcttatcttacaactccc

SEQ ID NO: 424 - DNA, 60 bp, Artificial sequence
  probe
gagactgccatacataatatatgacttcctagggatctgaaatccataaactaagagaaa

SEQ ID NO: 425 - DNA, 60 bp, Artificial sequence
  probe
cttggtcagtgaaaagatgctactatattgcttttgtcccaaagtgagtaaaatccccta

SEQ ID NO: 426 - DNA, 60 bp, Artificial sequence
  probe
agcgcattagcatagtaactcctttcagattttttggagggacgtttggaagtggcttac

SEQ ID NO: 427 - DNA, 60 bp, Artificial sequence
  probe
gagcgagactatgagaagatgtttggcacgaaatgccatggctgtgacttcaagatcgac

SEQ ID NO: 428 - DNA, 60 bp, Artificial sequence
  probe
agcacgaatgattctattaaagaaaatcattaggaagtggtagaaactttaaatcgcccc

SEQ ID NO: 429 - DNA, 60 bp, Artificial sequence
  probe
tgttaagtgttcggcagtaacctactttgttccttcgcctcagcagcaaatcttgctact

SEQ ID NO: 430 - DNA, 60 bp, Artificial sequence
  probe
taggctaaatttctccagcctcacaatggtcttcacttggtctgacttgtaccaattcta

SEQ ID NO: 431 - DNA, 60 bp, Artificial sequence
  probe
ggaacaatcattatacggactcttcagatttacagagaacttacacttcatctgttccac

SEQ ID NO: 432 - DNA, 60 bp, Artificial sequence
  probe
tttgtctgatatttagagacattcagattcagaagccatgagttcagttagctcatgggc

SEQ ID NO: 433 - DNA, 60 bp, Artificial sequence
  probe
ccttgaaaaatcattaaagccaaggtattaaaacctttgtgcattaataccttctagggg

SEQ ID NO: 434 - DNA, 60 bp, Artificial sequence
  probe
cttaggttccgtctcacggcgttttaatttattttcactgtcacacgcatagatccacga

SEQ ID NO: 435 - DNA, 60 bp, Artificial sequence
  probe
tgacacagatgaaccatattcccatgcacattgatcatgaccggaacatggactgggttt

SEQ ID NO: 436 - DNA, 60 bp, Artificial sequence
  probe
ttgccctagtcttcagaatggtcctgagaaaacatcactacttcgatgttctactttgct

SEQ ID NO: 437 - DNA, 60 bp, Artificial sequence
  probe
cagaattgaaagatccaacagatcattaccatgcgcagcttttagaggataacctttttg

SEQ ID NO: 438 - DNA, 60 bp, Artificial sequence
  probe
tccagtaaaataacagcaatgacaatgagatcgtcagtattattttcacatttccctgag

SEQ ID NO: 439 - DNA, 60 bp, Artificial sequence
  probe
tctaaaccaagagaagtgagacaaaccatctaatatctgtatcatgctattaataggctg

SEQ ID NO: 440 - DNA, 60 bp, Artificial sequence
  probe
gaacgctttgagacaacctactttaagaagtttcctggatactatgttacaggagatggc

SEQ ID NO: 441 - DNA, 60 bp, Artificial sequence
  probe
gctgtgcaacagacaggtgatgatgatgaagtagatgactttgaggacttcatcttcagc

SEQ ID NO: 442 - DNA, 60 bp, Artificial sequence
  probe
tgtcgtcgtcgaagtagatgactttgaggacttcatcttcagccacttctttggagacaa

(19) United States
(12) Patent Application Publication
(10) Pub. No.: US 2009/0220956 A1
(43) Pub. Date: Sep. 3, 2009

(54) Prediction of Local Recurrence of Breast Cancer

(76) Inventor: Dimitry Serge Antoine Nuyten, Branford, CT (US)

Correspondence Address: TRASKBRITT, P.C., P.O. BOX 2550, SALT LAKE CITY, UT, 84110, US

(21) Appl. No.: 12/084,012

(22) PCT Filed: Oct. 25, 2006
(86) PCT No.: PCT/NL2006/000535

§ 371 Date: Aug. 5, 2008

(30) Foreign Application Priority Data
Oct. 25, 2005 (EP) 05077456.1

(51) Int. Cl. C12Q 1/68

(52) U.S. Cl. 435/6

(57) ABSTRACT

The invention relates to the field of medicine, in particular to cancer, more specifically breast cancer, most specifically to a method to predict the local recurrence of breast cancer after breast conserving therapy. It has been demonstrated that a classification on basis of the similarity of the gene expression profile to the gene expression profile of (serum) activated fibroblasts is able to distinguish significantly in risk of local recurrence in breast cancer patients.

### FIELD OF THE INVENTION

The invention relates to the field of medicine, in particular to cancer, more specifically breast cancer, most specifically to a method to predict the local recurrence of breast cancer after breast conserving therapy.

### INTRODUCTION AND STATE OF THE ART

Breast cancer is the leading cause of death in women and its occurrence has been gradually increasing in the western world over the last 30 years. Several types of therapy exist nowadays, of which removal of the tumor tissue is the most prominent, which removal can be as radical as complete mastectomy, with or without the adjacent lymph nodes. Also radiotherapy is applied in most cases (after mastectomy radiotherapy is applied in selected cases primarily based on tumor size, presence of angioinvasion and lymph node status), either alone (only in very advanced tumors surgery is omitted) or in combination with surgical removal of the tumor tissue. Nowadays, mostly for esthetical reasons it is tried to achieve as much as possible breast conserving therapy, i.e. preventing a total mastectomy by precise surgery of the tumor tissue and precise radiotherapeutical treatment of the remaining surrounding tissue (entire breast; always) and lymph nodes (selectively) either or not in combination with chemotherapy.

Breast conserving therapy is defined as excision of the primary tumor and a tumor free margin, followed by radiation therapy (XRT) of the whole breast or the breast and regional lymph nodes. If chemotherapy is not to be given, XRT should be started in a timely fashion after conservative surgery is performed (usually within 2 to 4 weeks). XRT may be delayed if significant seroma is present, if a mastitis is present, if aim range of motion is still limited, or if incisions are not healed. The best way to integrate XRT and chemotherapy in patients who are to receive both is not yet well defined. The two modalities have been given concurrently, sequentially, or in a sandwich fashion (i.e., chemotherapy both prior to and after XRT). Often all or a portion of chemotherapy is given initially.

Megavoltage radiation therapy is recommended to the whole breast using tangential fields (without bolus) treating to a dose of 50 Gy (1.8 to 2 Gy per fiaction) over a 4½ to 5½ week period. This is usually followed by a boost of XRT to the area of the excisional biopsy for an additional 10 to 26 Gy. Omission of the boost may be associated with an increased risk of breast cancer recurrence, even in patients with negative margins and especially in patients below the age of 40.

Regional (lymph node) radiotherapy is sometimes performed after breast conserving surgery including a level I/level II axillary lymph node dissection. Regional radiotherapy is controversial but frequently considered for patients with positive axillary lymph nodes (>3), a positive high axillary lymph node, extranodal disease extension (doubtful), or a large axillary lymph node; or if <6 lymph nodes were removed from the axilla without the aid of sentinel lymph node biopsy. When done, regional XRT may include the supraclavicular, axillary, and internal mammary chain areas.

However, still between 10% aid 40% of women with operable breast cancer will experience an isolated locoregional recurrence following their primary treatment. There is currently no good evidence that adjuvant systemic treatment is effective in this situation and there is no standard treatment for women who have such a recurrence. After breast conserving therapy, treatment of a local recurrence often results in a so-called “salvage mastectomy”, optionally followed by radiotherapy or hyperthermia. If a local recurrence is combined with a general relapse by metastases the treatment will be determined on a case-by-case basis, but will in any case comprise hormonal or chemotherapy.

Radiotherapy regimens are, dependent on the characteristics of the primary tumor, able to produce two-thirds reduction in local recurrence and without long-term hazard, would be expected to produce an absolute increase in 20-year survival of about 2-4% (except for women at particularly low risk of local recurrence).

Breast cancer is a complex disease. Accumulation and combination of genetic and epigenetics anomalies cause tumorogenesis, genetic instability and acquisition of an increasingly invasive and resistant phenotype. This combinatorial origin and the heterogeneity of malignant cells, and the variety of the host background, create molecularly distinct subgroups of tumors endowed with different responses to therapy and clinical outcome. One of these factors is the recurrence of breast tumors after breast conserving therapy.

Conventional biological techniques have so far successfully identified some mechanisms of oncogenesis and indicated altered key genes such as ERBB2, P53, BRCA1 and BRCA2. Today, however, the elucidation of the human genome and technological developments have made it possible to simultaneously analyze the activity of many, if not all, genes in biological samples. Among the merging technologies, DNA (cDNA or oligo) arrays are currently prominent. DNA arrays tackle the complex and combinatorial nature of breast cancer genetics and offer an opportunity to confront the tumor heterogeneity. Key genes have been discovered by comparing the gene expression profiles of different (groups of) patients of normal and carcinomic tissues. Some of the differentially expressed genes are known to be involved in mammary oncogenesis (e.g. ERBB2 and MUC1). For others, such as GATA3, the correlation is unexpected and calls for further investigation (Bertucci, F. et al., Hum. Mol. Genet. (2000), 9:2981-2991).

Comprehensive gene expression profiles can be used to discriminate new relevant subclasses (see Perou, C. M. et al., (2000) Nature 406:747-752, and Sorlie, T. et al. (2001) Proc. Natl. Acad. Sci. USA 98:10869-10874) within classes of clinically indistinguishable tumors. Such stratification, together with the increasing availability of new alternative diagnostic and therapeutic options, is expected to guide the clinicians in choosing the most appropriate therapeutic strategy. Two examples which have emerged can be given here.

Bertucci et al. (supra) have been able by analyzing the expression of about 200 genes to identify 2 groups with different survival from a cohort of patients having a tumor with poor prognosis treated with chemotherapy (anthracyclin). They were able to identify 23 genes which were differentially expressed, and can now predict which patients from the cohort would benefit from anthracyclin chemotherapy.

Secondly, Van't Veer et al. (Nature (2002) 415:530-536, and WO 2004/065545) found a set of 70 genes (among an array of about 25,000 genes) which could distinguish between patients with or without 5 years metastasis-free survival, thereby preventing secondary or overlong treatments for patients without need for them. This profile has been validated by Van de Vijver et al (NEJM 2002) on a series of 295 patients, both lymph node negative and positive.

Recently, it has been demonstrated (Chang, H. Y., et al. (2004) PLoS Biol. 2(2):e7, to be found at: http://www.pubmedcentral.nih.gov/articlerender.fcgi?artid=314300) that a similar distinction could be made on another set of genes which relate to wound healing. However, of this set of 512 genes only 11 genes were in common with the set of Van't Veer.

All these profile focus on predicting distant metastasis free and/or overall survival.

Yet, there is still a need for a distinction between groups of breast cancer patients which have undergone breast conserving therapy with respect to the breast tumor recurrence chance (so called local recurrence).

### SUMMARY OF THE INVENTION

This invention now provides a method to predict the risk of local recurrence of breast cancer in patients having received breast conserving therapy comprising the steps of

a. measuring a wound signature gene expression profile of a patient; and
b. classifying said profile as “activated” or “quiescent”, wherein a classification as “activated” indicates a high risk on local recurrence. Preferably in such a method the wound signature expression profile comprises the expression profile of at least about 60%, preferably about 70%, more preferably about 80%, more preferably about 90%, more preferably about 95%, more preferably about 99%, most preferably all of the 442 genetic elements listed in Table 1. Alternatively, the wound signature expression profile comprises at least the expression profile of the top 200, preferably the top 250, more preferably the top 300, more preferably the top 350, more preferably the top 400, more preferably the top 425, more preferably the top 440 of the 442 genetic elements listed in Table 1.

Another aspect of the invention is the use of a wound signature gene set for the determination of the risk on local recurrence in breast cancer patients treated with breast conserving therapy, wherein preferably the wound signature gene set comprises at least about 60%, preferably about 70%, more preferably about 80%, more preferably about 90%, more preferably about 95%, more preferably about 99%, most preferably all of the 442 genetic elements listed in Table 1, or, alternatively comprises at least the top 200, preferably the top 250, more preferably the top 300, more preferably the top 350, more preferably the top 400, more preferably the top 425, more preferably the top 440 of the 442 genetic elements listed in Table 1

### DESCRIPTION OF THE FIGURES

FIG. 1 shows the local recurrence free survival (according to Kaplan-Meier) for a group of breast cancer patients, which have been classified according to the method of the invention. The x-axis represents the number of years, while the y-axis represents the percentage (divided by 100) of patients.

### DETAILED DESCRIPTION OF THE INVENTION

Chang et al. (supra) described the identification of a wound healing gene signature (identified in their publication as fibroblast core serum response (CSR) signature). This signature has been derived from wound healing tissue from normal fibroblasts. It was shown by Chang and co-workers that the molecular features that define the wound-type phenotype can predict an increased risk of metastasis and death in breast and other carcinomas. It appeared that patients with a so-called “activated Wound Signature” have a relatively poor outcome.

Now, surprisingly, it is shown by the present inventors that the wound signature genes, can also be used to predict an increased risk of local recurrence in breast tumors.

“Local recurrence” or “recurrence” as used in this specification means the outgrow of new or therapy resistent tumor cells on or from the spot of the first, treated tumor. It is different from metastasis, since metastasis normally requires spreading—e.g. through circulation or lymph tissue—of tumor cells which can trigger de novo tumor formation, while local recurrence does not require spread.

“Wound signature genes” or “wound healing signature genes” as used herein means the set of genes identified by Chang et al. (supra) and indicated by these authors also as CSR signature gene set. The set comprises 512 genes, which were present on the cDNA array used by Chang as 573 clones. Of these 512 genes 459 are uniquely identified in UniGene (http://www.ncbi.nlm.nih.gov/entrez/querv.fcgi?db=unigene).

As shown in the experimental data it surprisingly appeared possible to predict local recurrence on basis of the expression data of the Wound Signature genes. A local recurrence profile was developed on a randomly selected training set and tested on a validation set containing gene expression profiles of patients who remained free of local recurrence or who had had a local recurrence in the—median—7.8 years follow up period.

As indicated the Wound Signature gene set has been defined by Chang et al. (supra) according to their observation that wounds share many features with cancerous outgrowths. To come to their selection Chang et al. used a microarray of human cDNA containing approximately 43,000 elements, representing approximately 36,000 different genes. From a set of 50 fibroblast cultures derived from ten anatomic sites they identified the common serum response in fibroblasts by checking for high and low expressing genes, to find genes that were coordinately induced or repressed in transcriptional response to stimulation with serum. The thus obtained set was corrected by comparison with a set of genes periodically expressed during the HeLa cell cycle and skipping the overlapping clones. This resulted in a set of 512 genes (represented by 573 clones on the array).

In the present invention, an oligomicroarray as defined by van't Veer et al. (supra) was used. This set consisted of about 24,496 clones (Hu25K microarray) and is described on page 146-147 of WO 2004/065545, which description is herein incorporated by reference. It appeared that 442 clones of the wound signature gene set are present on this array. These are listed in Table 1.

To investigate a gene expression profile for these 442 clones, the array should be subjected to hybridisation with target polynucleotide molecules from a clinically relevant source, in this case e.g. a person with breast cancer having had breast conserving therapy. Therefore a fresh frozen (within 1 hour from surgical removal), liquid nitrogen (−80° C.) stored tumor sample needs to be available. Said target polynucleotide molecules should be expressed RNA or a nucleic acid derived thereform (e.g., cDNA or amplified RNA derived from cDNA that incorporates an RNA polymerase promoter). If the target molecules consist of RNA, it may be total cellular RNA, poly(A)+messenger RNA (mRNA) or fraction thereof, cytoplasmic mRNA, or RNA transcribed from cDNA (cRNA). Methods for preparaing total and poly(A)+messenger RNA are well known in the art, and are described e.g. in Sambrook et al., Molecular Cloning-A Laboratory Manual (2^{nd} Ed.) Vols. 1-3, Cold Spring Harbor, N.Y. (1989). In one embodiment, RNA is extracted from cells using guanidinium thiocyanate lysis followed by CsCl centrifugation (Chrigwin et al., (1979) Biochem. 18:5294-5299). In another embodiment, total RNA is extracted using a silica-gel based column, commercially available examples of which include RNeasy (Qiagen, Valencia, Calif., USA) and SrataPrep (Stratagene, La Jolla, Calif., USA). Poly(A)⁺ messenger RNA can be selected, e.g. by selection with oligo-dT cellulose or, alternatively, by oligo-dT primed reverse transcription of total cellular RNA. In another embodiment, the polynucleotide molecules analyzed by the invention comprise cDNA, or PCR products of amplified RNA or cDNA.

The sample comprises breast cells from a normal individual (i.e., an individual not afflicted with breast tumor). Such a sample can be used for control hybridization experiments or to establish a baseline level. The sample may also be derived from collected samples from a number of normal individuals. In a preferred embodiment, the sample comprises breast cells from a person with breast cancer having had breast conserving therapy. In a further preferred embodiment, a collection of samples is used taken from a number of individuals having breast tumors. Said sample preferably comprises breast cancer cells, or cells that are suspected of being breast cancer cells. In yet another embodiment, the collection is derived from normal or breast cancer cell lines or cell line samples. If applicable, it is preferred that the percentage of tumor cells in a sample is more than 80%, more preferred more than 40%, more preferred more than 50%, more preferred more than 60%, more preferred more than 70%, more preferred more than 80%, more preferred more than 90%, or 100% of the total number of cells.

Preferably, the target polynucleotides are detectably labelled at one or more nucleotides, Any method known in the art may be used to detectably label the nucleotides. Preferably, this labelling incorporates the label uniformly along the length of the polynucleotide and is carried out at a high degree of efficiency. One embodiment for this labelling uses oligo-dT primed reverse transcription to incorporate the label; however, conventional methods hereof are biased toward generating 3′ end fragments. Thus, in a preferred embodiment, random primers (e.g. 9-mers) are used in reverse transcription to uniformly incorporate labelled nucleotides over the full length of the target polynucleotides. Alternatively, random primers may be used in conjunction with PCR methods or T7 promoter-based in vitro transcription methods in order to amplify the target polynucleotides.

In a preferred embodiment, the detectable label is a luminescent label. For example, fluorescent labels, bioluminescent labels, chemiluminescent labels and calorimetric labels may be used. In a highly preferred embodiment, the label is a fluorescent label, such as a fluorescein, a phosphor, a rhodamine, or a polymethine dye or derivative. In another embodiment, the detectable label is a radiolabelled nucleotide.

Nucleic acid hybridisation and wash conditions are chosen so that the target polynucleotide molecules specifically hybridize to the complementary polynucleotide sequences of the array, preferably to a specific array site, wherein its complementary DNA is located. Optimal hybridisation conditions will depend on the type (e.g., RNA or DNA) of the target nucleotides. General parameters for specific (i.e., stringent) conditions of hybridisation are described in Sambrook et al. (supra). Typical hybridisation conditions for cDNA microarrays are hybridisation in 5×SSC plus 0.2% SDS at 65 DC four hours, followed by washes at 25° C. in low stringency wash buffer (1×SSC plus 0.2% SDS), followed by 10 minutes at 25° C. in higher stringency wash buffer (0.1×SSC plus 0.2% SDS).

When fluorescently labelled probes are used, the fluorescence emissions at each site of the microarray may be detected by scanning confocal laser microscopy. In one embodiment, the arrays is scanned with a laser fluorescent scanner with a computer controlled X-Y stage and a microscope objective. Fluorescent laser scanning devices are described in e.g. Schena et al. (1996) Genome Res. 6:639-645. Signals are recorded and, in a preferred embodiment, analysed by computer using a 12 or 16 bit analog to digital board. In one embodiment the scanned image is despeckled using a graphics program (e.g., Hijaak Graphics Suite) and then analysed using an image gridding program that creates a spreadsheet of the average hybridisation at each wavelength at each site.

Using the above described general analysis method, the expression profile of the Wound Signature genes in a biological sample (e.g., a biopsy) can be assessed. Using a large group of patients Chang et al. found a biphasic distribution of expression profiles of the Wound Signature genes in breast cancer tumors, and they accordingly identified two groups of tumors: one with a so-called quiescent expression profile and the other with a so-called activated expression profile, wherein the activated profile was highly correlated with metastasis and poor overall survival. In the present invention we have now shown that an expression profile of the Wound Signature genes is highly correlated with local recurrence of breast tumors.

Therefore, the invention relates to a method to predict the risk of local recurrence of breast cancer tumors in patients having received breast conserving therapy comprising the steps of measuring a wound signature gene expression profile of a patient, and classifying said profile as “activated” or “quiescent”, wherein a classification as “activated” indicates a high risk on local recurrence.

The invention thus also relates to a method for determining a wound signature gene expression profile for local recurrence of breast cancer, comprising determining the expression profile of at least the top two hundred of the genes listed in Table 1 in a breast tumor sample from at least one patient with local recurrence; determining the expression profile from the genes in a breast tumor sample from at least one patient without local recurrence; and determining from said expression profiles an “activated” and/or a “quiescent” expression profile, wherein said activated profile is the average and/or mean of said at least one and preferably at least 4 and more preferably at least 9 or even more preferred at least 17 patients that showed local recurrence, and wherein said “quiescent” profile is the average and/or mean of said at least one and preferably at least 30, preferably at least 60, more preferably at least 81 and even more preferred 144 patients that did not show local recurrence. A test sample comprising cells from a subject for which the risk of local recurrence is to be determined is used to determine the test profile on the at least top 200 genes of table 1 whereupon the obtained test profile is classified as being from a patient with a high or a low risk by comparing said test profile with said “activated” and/or said “quiescent” profile. Preferably said test profile is compared with said “activated” profile.

For the comparison of the expression profiles, methods that are or will be known to a skilled person can be used, such as, but nor limited to, a Pearson correlation.

In a preferred embodiment, the method for determining the expression level from at least the top two hundred of the genes listed in Table 1 comprises the use of probes comprising nucleic acid sequences as listed in Table 1, or homologues thereof that are able to hybridize to the corresponding genes, such as homologues that are 80% or more, or 90% or more, identical to the nucleic acid sequences shown in Table 1. The probes may comprise DNA sequences, RNA sequences, or copolymer sequences of DNA and RNA The molecules may also comprise DNA and/or RNA analogues such as, for example nucleotide analogues or peptide nucleic acid molecules (PNA), or combinations thereof. The molecules may comprise full or partial fragments of genomic DNA. The molecules may also comprise synthesized nucleotide sequences, such as synthetic oligonucleotide sequences. The sequences can be synthesized enzymatically in vivo, or enzymatically in vitro (e.g. by PCR), or non-enzymatically in vitro.

Furthermore, the invention relates to a method for determining a wound signature gene expression profile for local recurrence of breast cancer, comprising hybridizing RNA or a derivative thereof obtained from a breast tumor sample to a set of nucleic acid molecules comprising probes for at least 200 of the genes listed in Table 1; and quantifying the hybridization signals obtained from the RNA or a derivative thereof to the probes.

The method for determining a wound signature gene expression profile for local recurrence of breast cancer may further comprise determining the mean expression value (centroid) for each of the hybridization signals to the probes.

In a further embodiment, the invention relates to a method for determining the risk for local recurrence in a breast tumor sample from a patient comprises determining a wound signature gene expression profile by hybridizing RNA or a derivative thereof obtained from a breast tumor sample from the patient with a set of nucleic acid molecules comprising probes for at least the top 200 of the genes listed in Table 1; comparing the profile with the profile obtained from a breast tumor sample with local recurrence; and determining from the comparison whether the patient is at high risk for local recurrence.

In a method for determining the risk for local recurrence, a Pearson correlation of the mean expression value can be used for comparing the profiles

Not all of the genes are evenly contributing to the discriminating effect. As is shown in Table 1, the genes differ in significant expression. Although the statistical data presented in the Example are calculated with all of the 442 genetic elements of Table 1, it is submitted that a good distinction between the two groups of patients and therewith a good predicting ability of the wound signature gene set can also be achieved with only a part of the elements of Table 1, Preferably 60% of the elements of Table 1 are included in the analysis, more preferably 70%, more preferably 80%, more preferably 90%, more preferably 95%, more preferably 99% and most preferably all of the elements. It would be advisable not to randomly choose the elements, but to pick the top 200, top 250, top 300, top 350, top 400, top 425, top 440 or all 442 elements listed in Table 1. Of course also other genetic elements which are linked to the fibroblast response to serum-stimulus may be included in the analysis. It is even predicted that such ‘novel’ elements, which have not been included in the present analysis because of the chance nature of the DNA arrays used, will have a significant expression which can rank at any place in Table 1.

Using the microalray as described in the Example (obtainable from Rosetta Inpharmatics LLC, Kirkland, US), together with the prescribed analysis method the risk of local recurrence of a tumor for any breast cancer patient treated with breast conservative therapy can be obtained. Calibration will generally be obtained by referring the gene expression profile of one patient to a control sample consisting of a pooled sample of a large number of breast cancer patients (see FIG. 2 of WO 2004/065545). After measuring the expression profile of the wound signature gene set, the Pearson correlation with respect to the centroid data of Table 1 has to be calculated. If this value is higher than the cut-off level (Pearson correlation value higher than 0.3233) the patient runs a high risk of local recurrence.

If another microarray than specified above is used, it is advisable to calibrate the profiling analysis by measuring a number of samples according to the method described above to establish the baseline level (i.e. the quiescent profile). This can easily be done by taking a sample from a non-tumerous and non-wound healing tissue. Such a ‘normal’ tissue should approximately give an expression profile which resembles the quiescent fibroblast state (see also FIG. 2 of Chang et al.). The figures that are obtained can be compared to the figures presented by Chang et al., and/or the figures from the expression levels in Table 1, to have an indication of the differences caused by the analysis method. It will then be possible to calculate the Pearson correlation with respect to the centroid values of Table 1, and to see whether this value exceeds the threshold value of 0.3233. If a higher correlation is diagnosed, the patient will run an increased risk of local recurrence and therapy can be adjusted to cope with this prospect. Alternatively, or if the analysis measurements differ too much from the expression data presented in Table 1, a new centroid value should be calculated for those specific analysis conditions. For this a set of patients should be taken, with a known local recurrence, and the mean expression data for each gene from the wound signature gene set should be taken (see the Example for more details). Also, a new optimal cut-off value should then be established on the Pearson correlation data obtained with these new centroid values.

### Example

In a previously described series of 295 stage I and II breast carcinomas treated at the Netherlands Cancer Institute (Van't Veer et al, supra; also page 164 ff of WO 2004/065545) gene expression data of about 25,000 genetic elements have been obtained using oligonucleotide microarray technology. 161 patients were treated with breast conserving therapy, of which 17 did show a local recurrence (LR) in the years that they were followed. The median follow-up was 7.8 years; the LR-free rate at 10 years for the entire group is 85%

The elements listed in Table 1 (wound signature genes) were identified on the microarray and an unsupervised hierarchical clustering (see Example 1 of WO 2004/066545) was used on the expression profiles for these genes to determine an activated or quiescent wound signature.

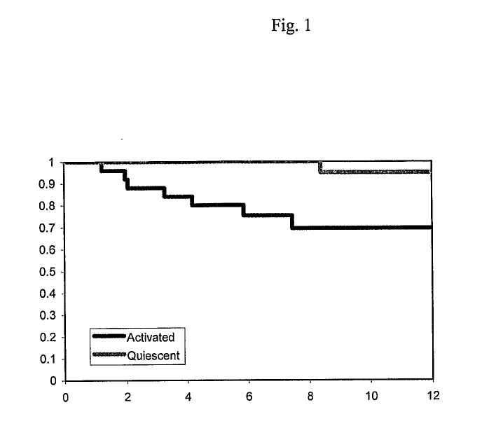

To further optimise the prognostic performance a training group (n=81 of which 9 LR) was defined and the expression profile of these patients was analysed. The mean expression value of the 9 LR patients was calculated for each gene (LR centroid value, see Table 1). Then for each of the 81 patients the Pearson correlation with the centroid value was calculated for the wound signature genes. A cut off value which would optimally discriminate between the two groups was determined (at Pearson correlation value=0.3233). This threshold was subsequently used to study risk of local recurrence in the validation set (n=80, 8 LR). Patients were classified as having an activated (high risk for local recurrence) or a quiescent (low risk for local recurrence) signature, based on a Pearson correlation value of above or below 0.3233, respectively. It appeared that the LR rate at 10 years in the validation series (see FIG. 1 and Table 2) was 95% for the group classified as activated and 69.5% for the group classified as quiescent (p=0.0005). FIG. 1 is a graphical representation of the local recurrence free survival score (according to Kaplan-Meier) for the validation series. The sensitivity in the validation set is 87.5% (⅞) with a specificity of 75% (54/72). Table 2 shows the data for the validation series from the Cox-regression model wherein the classifier (result from the Pearson correlation on the wound signature gene set) is weighted against known “historical” risk factors for a local recurrence. As shown the classifier is the only significant predictor for local recurrence (very low p-value (second column). The 3^{rd} column indicates the hazard ratio or relative risk. So a patient with an activated (high risk for local recurrence) signature has a 23 fold risk at a local recurrence compared with a patient with a quiescent signature (low risk for local recurrence). The last 2 columns indicate the 95% Confidence interval. More details on the experimental procedure and the statistical analyses which can be used (e.g., Kaplan-Meier and Cox-regression analyses) can be found in WO 2004/065645 (whole text and specifically example 10). It thus appears that a wound signature gene set can be used to distinguish significantly in risk of LR after breast conserving therapy.

TABLE 1

List of genes of the wound signature gene set. UniqID is the systemic name
of the sequence on the array.

| | | | | | | SEQ |
|---|---|---|---|---|---|---|
| | Gene | | Order_ | Unigene_ | Unigene_ | ID |
| UniqID | Symbol | Centroid | Sig | 172_158 | 158 | # |
| NM_001615 | ACTG2 | −0.5383953 | 56 | Hs.403989 | Hs.378774 | 1 |
| NM_001647 | APOD | −0.1826098 | 57 | Hs.75736 | Hs.75736 | 2 |
| NM_004265 | FADSD6 | −0.2060959 | 72 | Hs.388164 | Hs.184641 | 3 |
| NM_001710 | BF | −0.2874178 | 163 | Hs.69771 | Hs.69771 | 4 |
| NM_014585 | SLC11A3 | −0.1806041 | 171 | Hs.409875 | Hs.5944 | 5 |
| NM_002345 | LUM | −0.1596883 | 300 | Hs.406475 | Hs.79914 | 6 |
| AL133644 | | −0.1037048 | 311 | Hs.179735 | Hs.179735 | 7 |
| AF113007 | DKFZP586 | 0.011318 | 323 | Hs.288771 | Hs.288771 | 8 |
| | AD522 | | | | | |
| NM_003878 | GGH | 0.1060228 | 408 | Hs.78619 | Hs.78619 | 9 |
| Contig34896_RC | | −0.0777164 | 416 | Hs.8739 | Hs.170261 | 10 |
| D25328 | PFKP | −0.2891304 | 432 | Hs.26010 | Hs.99910 | 11 |
| NM_002318 | LOXL2 | −0.1697346 | 516 | Hs.83354 | Hs.83354 | 12 |
| NM_020372 | LOC57100 | −0.1427509 | 549 | Hs.373498 | Hs.373498 | 13 |
| AL137274 | MIG-6 | −0.0794952 | 585 | Hs.11169 | Hs.11169 | 14 |
| Contig56705_RC | | −0.0906733 | 607 | Hs.380763 | Hs.60293 | 15 |
| Contig54295_RC | | 0.0499989 | 630 | Hs.500350 | Hs.44829 | 16 |
| NM_000210 | ITGA6 | 0.0304433 | 632 | Hs.212296 | Hs.227730 | 17 |
| Contig57062_RC | | −0.0168056 | 676 | Hs.255149 | Hs.25253 | 18 |
| U95006 | | −0.0695112 | 709 | Hs.37616 | Hs.37616 | 19 |
| NM_005063 | SCD | −0.0116634 | 736 | Hs.119597 | Hs.119597 | 20 |
| NM_006074 | STAF50 | 0.0569467 | 738 | Hs.318501 | Hs.318501 | 21 |
| NM_004585 | RARRE53 | −0.058851 | 762 | Hs.17466 | Hs.17466 | 22 |
| NM_003129 | SQLE | 0.0171384 | 826 | Hs.71465 | Hs.71465 | 23 |
| NM_016348 | C5ORF4 | −0.1066377 | 841 | Hs.10235 | Hs.10235 | 24 |
| AL133074 | | −0.0381854 | 845 | Hs.75497 | Hs.75497 | 25 |
| NM_000076 | CDKN1C | 0.0668578 | 852 | Hs.106070 | Hs.106070 | 26 |
| NM_001993 | F3 | −0.2427101 | 856 | Hs.62192 | Hs.62192 | 27 |
| NM_003379 | VIL2 | 0.0300496 | 895 | Hs.403997 | Hs.155191 | 28 |
| NM_000784 | CYP27A1 | −0.1121366 | 911 | Hs.82568 | Hs.82568 | 29 |
| X59405 | MCP | −0.0561621 | 920 | Hs.83532 | Hs.83532 | 30 |
| M55914 | MPB1 | −0.0370872 | 926 | # N/A | Hs.254105 | 31 |
| NM_005694 | COX17 | −0.0719567 | 976 | Hs.16297 | Hs.16297 | 32 |
| Contig47563_RC | | 0.1292021 | 1005 | # N/A | Hs.30818 | 33 |
| AB040884 | KIAA1451 | −0.0674023 | 1034 | Hs.109694 | Hs.109694 | 34 |
| AF035284 | | −0.0321418 | 0153 | Hs.503546 | Hs.132898 | 35 |
| NM_016423 | ZNF219 | −0.0716907 | 1066 | Hs.250493 | Hs.250493 | 36 |
| NM_000484 | APP | −0.0036143 | 1080 | Hs.177486 | Hs.177486 | 37 |
| NM_001386 | DPYSL2 | 0.0356441 | 1083 | Hs.173381 | Hs.173381 | 38 |
| NM_003090 | SNRPA1 | 8.69E−04 | 1085 | Hs.434901 | Hs.80506 | 39 |
| NM_016058 | LOC51002 | −0.0520914 | 1091 | Hs.157401 | Hs.433212 | 40 |
| NM_003255 | TIMP2 | −0.0888242 | 1110 | Hs.6441 | Hs.6441 | 41 |
| NM_005648 | TCEB1 | −0.0206109 | 1113 | Hs.435169 | Hs.184693 | 42 |
| AL050002 | | 0.0040601 | 1122 | Hs.357004 | Hs.94795 | 43 |
| NM_000214 | JAG1 | −9.07E−04 | 1131 | Hs.409202 | Hs.91143 | 44 |
| AB029018 | KIAA1095 | −0.072232 | 1146 | Hs.177635 | Hs.177635 | 45 |
| Contig37878_RC | | 0.0955788 | 1179 | Hs.440973 | Hs.255416 | 46 |
| D43950 | KIAA0098 | 0.0321762 | 1231 | Hs.1600 | Hs.1600 | 47 |
| NM_000366 | TPM1 | −0.0160874 | 1232 | Hs.133892 | Hs.77899 | 48 |
| NM_004126 | GNG11 | −0.0012581 | 1235 | Hs.83381 | Hs.83381 | 49 |
| Contig2930_RC | DAB2 | −0.0136946 | 1247 | Hs.81988 | Hs.291804 | 50 |
| NM_000235 | LIPA | 0.0196271 | 1258 | Hs.85226 | Hs.85226 | 51 |
| NM_001428 | ENO1 | −0.0117356 | 1294 | Hs.433455 | Hs.254105 | 52 |
| NM_003670 | BHLHB2 | −0.1595493 | 1298 | Hs.171825 | Hs.171825 | 53 |
| AK000060 | AK000060 | −0.1911419 | 1311 | Hs.388877 | Hs.27973 | 54 |
| NM_003094 | SNRPE | −0.0917718 | 1323 | Hs.334612 | Hs.334612 | 55 |
| NM_002997 | SDC1 | −0.0861853 | 1384 | Hs.82109 | Hs.82109 | 56 |
| NM_006813 | B4-2 | −0.0940814 | 1390 | Hs.75969 | Hs.75969 | 57 |
| NM_012385 | P8 | −0.107813 | 1417 | Hs.418692 | Hs.424279 | 58 |
| NM_002444 | MSN | −0.0408763 | 1478 | Hs.170328 | Hs.170328 | 59 |
| U82987 | BBC3 | −0.0864452 | 1486 | Hs.87246 | Hs.87246 | 60 |
| AB007916 | FLJ13052 | 0.0224214 | 1488 | Hs.458492 | Hs.214646 | 61 |
| NM_003289 | TPM2 | −0.0545574 | 1524 | Hs.300722 | Hs.300772 | 62 |
| NM_004911 | ERP70 | −0.051383 | 1545 | Hs.93659 | Hs.93659 | 63 |
| NM_000877 | IL1R1 | −0.0408296 | 1575 | Hs.82112 | Hs.82112 | 64 |
| NM_006519 | TCTEL1 | 0.0062432 | 1591 | Hs.266940 | Hs.266940 | 65 |
| Contig57494_RC | | −0.0364058 | 1620 | Hs.406339 | Hs.289069 | 66 |
| NM_000269 | NME1 | 0.1873206 | 1624 | Hs.118638 | Hs.118638 | 67 |
| NM_018048 | FLJ10292 | −0.0022011 | 1637 | Hs.104650 | Hs.104650 | 68 |
| Contig3607_RC | | −0.1111468 | 1650 | # N/A | Hs.31297 | 69 |
| NM_006022 | TSC22 | 0.0097983 | 1669 | Hs.114360 | Hs.114360 | 70 |
| Contig3820_RC | LOC56898 | −0.1493656 | 1684 | Hs.124696 | Hs.124696 | 71 |
| Contig3695_RC | | 0.0152393 | 1760 | Hs.26670 | Hs.26670 | 72 |
| NM_003186 | TAGLN | −0.101256 | 1772 | Hs.410977 | Hs.433399 | 73 |
| Contig48249_RC | | −0.033877 | 1795 | Hs.386784 | Hs.8768 | 74 |
| AK001362 | | −0.0041243 | 1833 | Hs.173374 | Hs.173374 | 75 |
| NM_000222 | KIT | −0.0978341 | 1918 | Hs.81665 | Hs.81665 | 76 |
| NM_004107 | FCGRT | −0.0915406 | 1932 | Hs.111903 | Hs.111903 | 77 |
| NM_018455 | BM039 | −0.002616 | 1945 | Hs.283532 | Hs.283532 | 78 |
| AL049471 | | 0.0479844 | 1956 | Hs.12702 | Hs.12702 | 79 |
| NM_000177 | GSN | 0.0159066 | 1974 | Hs.446537 | Hs.290070 | 80 |
| NM_001948 | DUT | −0.0863181 | 1982 | Hs.367676 | Hs.367676 | 81 |
| S90469 | POR | −0.0056894 | 2038 | Hs.354056 | Hs.167246 | 82 |
| NM_014463 | LSM3 | −0.0528273 | 2117 | Hs.111632 | Hs.111632 | 83 |
| NM_016824 | ADD3 | 0.1132136 | 2124 | Hs.324470 | Hs.324470 | 84 |
| NM_002106 | H2AFZ | 0.0111531 | 2154 | Hs.119192 | Hs.119192 | 85 |
| Contig52945_RC | | 0.0872467 | 2244 | Hs.172792 | Hs.172792 | 86 |
| NM_001070 | TUBG1 | −0.0609361 | 2253 | Hs.21635 | Hs.21635 | 87 |
| NM_006807 | CBX1 | 0.0275433 | 2307 | Hs.77254 | Hs.77254 | 88 |
| NM_002950 | RPN1 | 0.0069991 | 2389 | Hs.2280 | Hs.2280 | 89 |
| NM_002792 | PSMA7 | 0.038883 | 2429 | Hs.233952 | Hs.233952 | 90 |
| NM_004595 | SMS | 0.0183218 | 2433 | Hs.449032 | Hs.89718 | 91 |
| NM_006745 | SC4MOL | −0.0046231 | 2439 | Hs.393239 | Hs.239926 | 92 |
| Contig1998_RC | | 0.0184363 | 2458 | Hs.24758 | Hs.24758 | 93 |
| NM_002816 | PSMD12 | 0.1177832 | 2525 | Hs.4295 | Hs.4295 | 94 |
| NM_006291 | TNFAIP2 | −0.0699154 | 2533 | Hs.101382 | Hs.101382 | 95 |
| NM_005962 | MXI1 | −0.0582266 | 2594 | Hs.118630 | Hs.118630 | 96 |
| AF052159 | | 0.0228531 | 2614 | Hs.5957 | Hs.5957 | 97 |
| NM_012412 | PURB | −0.0055242 | 2642 | Hs.301005 | Hs.301005 | 98 |
| NM_016951 | HSPC224 | 0.0606148 | 2646 | Hs.15159 | Hs.15159 | 99 |
| NM_018639 | LOC55884 | 0.0719628 | 2649 | Hs.459470 | Hs.136644 | 100 |
| NM_002166 | ID2 | 0.0289007 | 2684 | Hs.180919 | Hs.180919 | 101 |
| NM_006114 | D1951177E | −0.0092836 | 2694 | Hs.310542 | Hs.30928 | 102 |
| Contig58471_RC | | −0.0827957 | 2726 | Hs.363138 | Hs.17567 | 103 |
| NM_004457 | FACL3 | 0.0088717 | 2741 | Hs.268012 | Hs.268012 | 104 |
| AK002174 | LOC51088 | −0.0255738 | 2764 | Hs.272251 | Hs.272239 | 105 |
| Contig2652_RC | | −0.0273396 | 2770 | Hs.436617 | Hs.184164 | 106 |
| Contig56768_RC | SLC5A3 | 0.0527617 | 2830 | Hs.268016 | Hs.268016 | 107 |
| NM_007043 | HRB2 | −0.0465499 | 2854 | Hs.269857 | Hs.154762 | 108 |
| AF052100 | | −0.0306271 | 2868 | Hs.6651 | Hs.6651 | 109 |
| NM_000791 | DHFR | 0.0658927 | 2887 | Hs.83765 | Hs.83765 | 110 |
| NM_001424 | EMP2 | −0.0693488 | 2893 | Hs.531561 | Hs.29191 | 111 |
| U47101 | NIFU | 0.0124747 | 2919 | Hs.350702 | Hs.9908 | 112 |
| Contig4574_RC | | −0.0165929 | 2928 | Hs.111099 | Hs.111099 | 113 |
| NM_014018 | HSPC007 | −0.0420779 | 2960 | Hs.55097 | Hs.55097 | 114 |
| Contig1505_RC | | −0.0468072 | 2979 | Hs.8345 | Hs.39504 | 115 |
| Contig45316_RC | | −0.0159842 | 2985 | Hs.5957 | Hs.5957 | 116 |
| NM_018281 | FLJ10948 | −0.0915379 | 3023 | Hs.170915 | Hs.34579 | 117 |
| NM_003944 | SELENBP1 | −0.079434 | 3129 | Hs.334841 | Hs.334841 | 118 |
| NM_017455 | SDFR1 | 0.0595179 | 3144 | Hs.389371 | Hs.389371 | 119 |
| AB029032 | KIAA1109 | −0.0509789 | 3156 | Hs.408142 | Hs.6606 | 120 |
| Contig37141_RC | | −0.0131654 | 3168 | Hs.483205 | Hs.432790 | 121 |
| NM_004906 | KIAA0105 | 0.0688316 | 3196 | Hs.446091 | Hs.119 | 122 |
| NM_005167 | ARHC | 0.0171709 | 3199 | Hs.179735 | Hs.179735 | 123 |
| Contig50396_RC | | −0.0021026 | 3356 | Hs.4094 | Hs.4094 | 124 |
| NM_003793 | CTSF | −0.0367427 | 3371 | Hs.11590 | Hs.11590 | 125 |
| NM_006347 | USA-CYP | −0.0609369 | 3381 | Hs.9880 | Hs.9880 | 126 |
| AB033034 | DKFZp762B226 | 0.011291 | 3402 | Hs.412128 | Hs.7041 | 127 |
| NM_001706 | BCL6 | −0.0877042 | 3427 | Hs.155024 | Hs.155024 | 128 |
| NM_016217 | LOC51696 | 0.0255174 | 3461 | Hs.6679 | Hs.6679 | 129 |
| AL049949 | | −0.0204419 | 3475 | Hs.28264 | Hs.28264 | 130 |
| NM_001482 | GATM | −0.1357806 | 3642 | Hs.75335 | Hs.75335 | 131 |
| NM_001551 | IGBP1 | −0.011337 | 3653 | Hs.3631 | Hs.3631 | 132 |
| NM_005360 | MAF | −0.0302207 | 3661 | Hs.134859 | Hs.30250 | 133 |
| NM_014454 | PA26 | 0.0255216 | 3672 | Hs.14125 | Hs.14125 | 134 |
| Contig52737_RC | | −0.14461 | 3708 | Hs.31297 | Hs.31297 | 135 |
| NM_006303 | JTV1 | 0.001045 | 3734 | Hs.301613 | Hs.301613 | 136 |
| NM_012428 | SDFR1 | 0.0758822 | 3739 | Hs.389371 | Hs.389371 | 137 |
| NM_016103 | LOC51128 | 0.0062434 | 3745 | Hs.279582 | Hs.279582 | 138 |
| NM_016065 | LOC51021 | −0.0124273 | 3815 | Hs.180312 | Hs.180312 | 139 |
| NM_020188 | DC13 | 0.0854393 | 3824 | Hs.6879 | Hs.6879 | 140 |
| NM_002185 | IL7R | 0.1228947 | 3856 | Hs.362807 | Hs.362807 | 141 |
| NM_000099 | CST3 | −0.0058468 | 3913 | Hs.304682 | Hs.304682 | 142 |
| NM_001152 | SLC25A5 | 0.0536659 | 3917 | Hs.79172 | Hs.79172 | 143 |
| NM_014669 | KIAA0095 | 0.0385347 | 3937 | Hs.295014 | Hs.155314 | 144 |
| NM_003093 | SNRPC | 0.0193292 | 4128 | Hs.1063 | Hs.1063 | 145 |
| NM_003115 | UAP1 | 0.0057854 | 4130 | Hs.21293 | Hs.21293 | 146 |
| NM_012333 | MYC8P | 0.0681289 | 4145 | Hs.78221 | Hs.78221 | 147 |
| NM_014909 | KIAA1036 | −0.0282497 | 4153 | Hs.155182 | Hs.155182 | 148 |
| NM_019903 | ADD3 | 0.100385 | 4163 | Hs.324470 | Hs.324470 | 149 |
| NM_001286 | CLCN6 | 0.0071871 | 4209 | Hs.371458 | Hs.211614 | 150 |
| NM_018287 | FLJ10971 | 0.0784592 | 4241 | Hs.396643 | Hs.13531 | 151 |
| AL133555 | CCT8 | 0.0567524 | 4252 | Hs.143736 | Hs.352413 | 152 |
| NM_004508 | IDI1 | 0.0725287 | 4295 | Hs.76038 | Hs.76038 | 153 |
| NM_000062 | SERPING1 | 0.0299511 | 4364 | Hs.384598 | Hs.151242 | 154 |
| NM_000373 | UMPS | −0.0990897 | 4366 | Hs.2057 | Hs.2057 | 155 |
| NM_021199 | CGI-44 | 4.57E−04 | 4403 | Hs.435468 | Hs.8185 | 156 |
| Contig54898_RC | | 0.0388843 | 4438 | Hs.424126 | Hs.356688 | 157 |
| NM_000788 | DCK | 0.0926807 | 4451 | Hs.709 | Hs.709 | 158 |
| NM_004301 | BAF53A | 0.0095139 | 4461 | Hs.435326 | Hs.274350 | 159 |
| NM_004593 | SFRS10 | 0.0762501 | 4463 | Hs.30035 | Hs.30035 | 160 |
| NM_005517 | HMG17 | 0.0691543 | 4467 | Hs.181163 | Hs.181163 | 161 |
| NM_017838 | FLJ20479 | 0.0029758 | 4634 | Hs.386392 | Hs.23990 | 162 |
| NM_018243 | FLJ10849 | −0.0218507 | 4636 | Hs.386784 | Hs.8768 | 163 |
| Contig55612_RC | | −0.0311422 | 4675 | Hs.267120 | Hs.267120 | 164 |
| Contig40105 | | −7.78E−07 | 4733 | Hs.350388 | Hs.350388 | 165 |
| NM_003091 | SNRPB | 0.0142074 | 4759 | Hs.83753 | Hs.83753 | 166 |
| Contig46583_RC | | −0.0198152 | 4825 | Hs.191320 | Hs.380474 | 167 |
| NM_012321 | LSM4 | 0.0303369 | 4870 | Hs.76719 | Hs.76719 | 168 |
| NM_014905 | GL5 | −0.095738 | 5041 | Hs.128410 | Hs.239189 | 169 |
| NM_016639 | FN14 | −0.0306102 | 5047 | Hs.355899 | Hs.355899 | 170 |
| Contig52717_RC | | 0.0127462 | 5084 | Hs.356618 | Hs.356618 | 171 |
| Contig58129_RC | | −0.0547341 | 5092 | Hs.163725 | Hs.163725 | 172 |
| AK002107 | | −0.1842058 | 5158 | Hs.123072 | Hs.20843 | 173 |
| NM_002687 | PNN | −0.1029911 | 5187 | Hs.409965 | Hs.44499 | 174 |
| NM_002808 | PSMD2 | 0.033058 | 5191 | Hs.388921 | Hs.74619 | 175 |
| NM_014325 | CORO1C | −0.0520972 | 5220 | Hs.17377 | Hs.17377 | 176 |
| AL110212 | PURB | 0.01098946 | 5244 | Hs.301005 | Hs.301005 | 177 |
| NM_003374 | VDAC1 | −0.0376429 | 5284 | Hs.404814 | Hs.149155 | 178 |
| L48692 | LOC56902 | 0.0353168 | 5354 | Hs.262858 | Hs.193384 | 179 |
| NM_001745 | CAMLG | −0.0106132 | 5360 | Hs.13572 | Hs.13572 | 180 |
| NM_002659 | PLAUR | −0.045088 | 5366 | Hs.179657 | Hs.179657 | 181 |
| U09579 | CPNE5 | −0.0288579 | 5403 | Hs.370771 | Hs.179665 | 182 |
| AL050353 | OIP2 | 0.0667026 | 5417 | Hs.274170 | Hs.274170 | 183 |
| NM_003707 | RUVBL1 | −0.1009717 | 5455 | Hs.272822 | Hs.272822 | 184 |
| NM_006397 | RNASEHI | 0.0568056 | 5469 | Hs.25292 | Hs.25292 | 185 |
| NM_020143 | LOC56902 | 0.0169222 | 5485 | Hs.262858 | Hs.193384 | 186 |
| Contig38493_RC | | 0.0565596 | 5511 | Hs.509629 | Hs.17767 | 187 |
| NM_002137 | HNRPA2B1 | 0.047456 | 5613 | Hs.232400 | Hs.232400 | 188 |
| NM_005805 | POH1 | 0.069186 | 5631 | Hs.178761 | Hs.178761 | 189 |
| NM_014350 | GG2-1 | 0.0541893 | 5644 | Hs.17839 | Hs.17839 | 190 |
| NM_018087 | FLJ10407 | −0.0295427 | 5657 | Hs.435982 | Hs.30738 | 191 |
| AF113020 | PRO2463 | 0.024994 | 5667 | Hs.406243 | Hs.90421 | 192 |
| AK001163 | ADE2H1 | 0.0522322 | 5673 | Hs.444439 | Hs.117950 | 193 |
| Contig57034_RC | | −0.001299 | 5796 | Hs.127337 | Hs.127337 | 194 |
| NM_002431 | MNAT1 | 0.0182942 | 5807 | Hs.72870 | Hs.433410 | 195 |
| NM_004461 | FARSL | 0.0126879 | 5815 | Hs.23111 | Hs.23111 | 196 |
| NM_007107 | SSR3 | 0.0756157 | 5826 | Hs.28707 | Hs.28707 | 197 |
| U09848 | ZNF36 | 0.0324513 | 5844 | Hs.528676 | Hs.356344 | 198 |
| NM_020401 | NUP107 | 0.0642592 | 5939 | Hs.355598 | Hs.236204 | 199 |
| NM_016271 | STRIN | 0.0660269 | 6140 | Hs.180403 | Hs.180403 | 200 |
| NM_002452 | NUDT1 | −0.0117806 | 6211 | Hs.413078 | Hs.388 | 201 |
| NM_018290 | FLJ10983 | −0.0173684 | 6256 | Hs.23363 | Hs.23363 | 202 |
| X66087 | MYBL1 | −0.0303777 | 6261 | Hs.300592 | Hs.300592 | 203 |
| Contig20651_RC | | 0.0977492 | 6363 | Hs.180403 | Hs.180403 | 204 |
| NM_016491 | MRPL37 | 0.0466628 | 6424 | Hs.4209 | Hs.4209 | 205 |
| D80010 | LPIN1 | −0.0185498 | 6473 | Hs.81412 | Hs.81412 | 206 |
| NM_006618 | PLU-1 | −0.0720389 | 6494 | Hs.143323 | Hs.143323 | 207 |
| AB020681 | KIAA0874 | −0.1063096 | 6522 | Hs.388877 | Hs.27973 | 208 |
| Contig41864_RC | | −0.0153596 | 6547 | Hs.188199 | Hs.15220 | 209 |
| NM_002949 | MRPL12 | −0.0402533 | 6583 | Hs.109059 | Hs.109059 | 210 |
| NM_005920 | MEF2D | −0.0251423 | 6594 | Hs.77955 | Hs.77955 | 211 |
| NM_003089 | SNRP70 | −0.008649 | 6684 | Hs.174051 | Hs.174051 | 212 |
| NM_016134 | LOC51670 | −0.0149143 | 6712 | Hs.197335 | Hs.197335 | 213 |
| NM_016326 | LOC51192 | 0.0542734 | 6717 | Hs.15159 | Hs.15159 | 214 |
| NM_005826 | HNRPR | 0.0151329 | 6817 | Hs.15265 | Hs.15265 | 215 |
| NM_012417 | RDGBB | 0.0476858 | 6826 | Hs.405933 | Hs.333212 | 216 |
| NM_016567 | TOK-1 | 0.0054519 | 6840 | Hs.337008 | Hs.279862 | 217 |
| D55716 | MCM7 | 0.0157898 | 6910 | Hs.438720 | Hs.77152 | 218 |
| NM_004953 | EIF4G1 | 0.002703 | 6934 | Hs.433750 | Hs.433750 | 219 |
| NM_006824 | P40 | 0.0316377 | 6944 | Hs.346868 | Hs.346868 | 220 |
| NM_014413 | HRI | 0.0059656 | 6950 | Hs.434986 | Hs.258730 | 221 |
| NM_019555 | ARHGEF3 | 0.0119673 | 6966 | Hs.25951 | Hs.25951 | 222 |
| AB033054 | KIAA1228 | 0.0195106 | 6973 | Hs.388073 | Hs.306867 | 223 |
| NM_004095 | EIF4EBP1 | 0.0021399 | 7044 | Hs.406408 | Hs.4333137 | 224 |
| NM_006833 | MOV34-34KD | −0.0384261 | 7062 | Hs.15591 | Hs.15591 | 225 |
| NM_012325 | MAPRE1 | −0.0165868 | 7070 | Hs.408754 | Hs.234279 | 226 |
| NM_016183 | LOC51154 | 0.015694 | 7076 | Hs.463797 | Hs.274201 | 227 |
| AB040969 | KIAA1536 | −0.0540262 | 7098 | Hs.156667 | Hs.156667 | 228 |
| Contig43506_RC | | 0.0382747 | 7128 | Hs.511765 | Hs.154762 | 229 |
| Contig719_RC | | 0.064509 | 7144 | Hs.120425 | Hs.22350 | 230 |
| NM_005956 | MTHFD1 | 0.0511302 | 7171 | Hs.435974 | Hs.172665 | 231 |
| NM_006938 | SNRPD1 | 0.0929607 | 7173 | Hs.86948 | Hs.86948 | 232 |
| NM_016310 | POLR3K | 0.029813 | 7184 | Hs.437186 | Hs.110857 | 233 |
| NM_016546 | LOC51279 | −0.021692 | 7304 | Hs.415792 | Hs.98571 | 234 |
| NM_016607 | ALEX3 | −0.0289851 | 7305 | Hs.172788 | Hs.172788 | 235 |
| X69111 | ID3 | 0.0048616 | 7319 | Hs.76884 | Hs.76884 | 236 |
| AF052183 | | −0.0091578 | 7326 | Hs.446551 | Hs.11039 | 237 |
| AK000685 | | −0.0520794 | 7330 | Hs.143601 | Hs.143601 | 238 |
| Contig3902_RC | | −0.0190224 | 7345 | Hs.26812 | Hs.26812 | 239 |
| NM_013300 | HSU79274 | −0.0264869 | 7415 | Hs.150555 | H.150555 | 240 |
| Contig47710_RC | | 0.0739388 | 7477 | Hs.98133 | Hs.98133 | 241 |
| NM_002004 | FDPS | 6.79E−04 | 7502 | Hs.335918 | Hs.335918 | 242 |
| NM_003051 | SLC16A1 | 0.03463 | 7645 | Hs.75231 | Hs.75231 | 243 |
| NM_003064 | SLPI | 0.0711473 | 7646 | Hs.251754 | Hs.251754 | 244 |
| NM_006452 | ADE2H1 | −0.0167971 | 7660 | Hs.444439 | Hs.117950 | 245 |
| Contig54752_RC | | 0.0036688 | 7767 | Hs.69476 | Hs.69476 | 246 |
| M94362 | LMNB2 | −0.0129366 | 7777 | Hs.76084 | Hs.76084 | 247 |
| Contig49652_RC | | 0.0642718 | 7924 | Hs.374421 | Hs.374421 | 248 |
| Contig56840_RC | | −0.0153949 | 7932 | Hs.8026 | Hs.8026 | 249 |
| NM_020185 | MKPX | −0.02206 | 8034 | Hs.29106 | Hs.29106 | 250 |
| NM_007021 | DEPP | −0.0060809 | 8158 | Hs.93675 | Hs.93675 | 251 |
| NM_014264 | STK1B | −0.0244607 | 8175 | Hs.172052 | Hs.172052 | 252 |
| AL133577 | | 0.0975647 | 8241 | Hs.106148 | Hs.106148 | 253 |
| NM_003016 | SFRS2 | 0.0251001 | 8340 | Hs.73965 | Hs.73965 | 254 |
| NM_002466 | MYBL2 | 0.028428 | 8500 | Hs.179718 | Hs.179718 | 255 |
| NM_006988 | ADAMTS1 | −0.0837017 | 8537 | Hs.8230 | Hs.8230 | 256 |
| NM_016391 | HSPC111 | 0.0234483 | 8552 | Hs.529475 | Hs.279918 | 257 |
| NM_017787 | FLJ20367 | −0.0563849 | 8557 | Hs.10346 | Hs.10346 | 258 |
| NM_017816 | FLJ20425 | 0.0125463 | 8559 | Hs.425427 | Hs.425427 | 259 |
| NM_021019 | MYL6 | −0.0399657 | 8568 | Hs.77385 | Hs.77385 | 260 |
| NM_002388 | MCM3 | 0.0231119 | 8700 | Hs.179565 | Hs.179565 | 261 |
| NM_003132 | SRM | 0.0567747 | 8706 | Hs.76244 | Hs.76244 | 262 |
| NM_004280 | EEF1E1 | 0.0296196 | 8712 | Hs.88977 | Hs.433779 | 263 |
| AB036063 | p53R2 | −0.018041 | 8785 | Hs.512592 | Hs.94262 | 264 |
| Contig20635_RC | | −0.0706198 | 8813 | Hs.412128 | Hs.7041 | 265 |
| Contig51797_RC | | 8.73E−04 | 8874 | Hs.42474 | Hs.108873 | 266 |
| NM_002184 | IL6ST | −0.0810843 | 8908 | Hs.71968 | Hs.82065 | 267 |
| NM_006534 | NCOA3 | 0.0349513 | 8942 | Hs.382168 | Hs.225977 | 268 |
| NM_016613 | LOC51313 | 0.0140286 | 8956 | Hs.323583 | Hs.323583 | 269 |
| NM_002971 | SATB1 | −0.0344931 | 9137 | Hs.416026 | Hs.74592 | 270 |
| NM_005785 | SBB103 | 0.0196583 | 9163 | Hs.436596 | Hs.153639 | 271 |
| NM_017768 | FLJ20331 | 0.0386201 | 9206 | Hs.283862 | Hs.50848 | 272 |
| NM_018381 | FLJ11286 | 0.0168729 | 9219 | Hs.175120 | Hs.12151 | 273 |
| U79458 | WBP2 | 0.0361998 | 9232 | # N/A | Hs.231840 | 274 |
| AF054996 | | −0.0422692 | 9245 | Hs.91579 | Hs.91579 | 275 |
| AL079279 | | −0.0035211 | 9257 | Hs.231971 | Hs.8963 | 276 |
| Contig24856_RC | | −0.0378088 | 9272 | Hs.9028 | Hs.9028 | 277 |
| Contig2493_RC | | 0.0189361 | 9273 | # N/A | Hs.256302 | 278 |
| Contig49757_RC | | 0.0468612 | 9321 | Hs.1600 | Hs.1600 | 279 |
| NM_004596 | SNRPA | 0.0022191 | 9403 | Hs.173255 | Hs.173255 | 280 |
| NM_006396 | SSSCA1 | 0.0027079 | 9421 | Hs.25723 | Hs.25723 | 281 |
| NM_007066 | PKIG | −0.0135997 | 9425 | Hs.3407 | Hs.3407 | 282 |
| AB037784 | KIAA1363 | 0.0220583 | 9482 | Hs.22941 | Hs.22941 | 283 |
| Contig37368_RC | | 0.1016643 | 9560 | Hs.3532 | Hs.17767 | 284 |
| NM_005704 | PTPRU | −0.0037938 | 9682 | Hs.19718 | Hs.19718 | 285 |
| NM_005796 | PP15 | 0.0747884 | 9683 | Hs.356630 | Hs.151734 | 286 |
| AB002321 | KIAA0323 | −0.0399421 | 9748 | Hs.7911 | Hs.7911 | 287 |
| AF055016 | LOC57213 | 0.0695341 | 9759 | Hs.44235 | Hs.44235 | 288 |
| Contig48842_RC | | −0.0020714 | 9863 | Hs.444449 | Hs.29088 | 289 |
| NM_001274 | CHEK1 | 0.0441693 | 9902 | Hs.24529 | Hs.20295 | 290 |
| NM_002692 | POLE2 | 0.0495898 | 9926 | Hs.99185 | Hs.99185 | 291 |
| NM_006444 | CAP-E | 0.0214551 | 9971 | Hs.119023 | Hs.119023 | 292 |
| NM_014977 | KIAA0670 | −0.0148572 | 10005 | Hs.227133 | Hs.227133 | 293 |
| NM_016126 | LOC51668 | 0.0123531 | 10012 | Hs.439171 | Hs.46967 | 294 |
| Contig53132_RC | | −0.001413 | 10219 | Hs.119023 | Hs.119023 | 295 |
| NM_000935 | PLOD2 | 0.0042861 | 10257 | Hs.41270 | Hs.41270 | 296 |
| M31212 | MYL6 | −0.0268881 | 10601 | Hs.77385 | Hs.77385 | 297 |
| NM_005928 | MFGE8 | −0.0444387 | 10696 | Hs.3745 | Hs.3745 | 298 |
| AK001025 | | 0.0331707 | 10821 | Hs.9081 | Hs.9081 | 299 |
| AL049435 | | −0.0408307 | 10831 | Hs.407903 | Hs.170056 | 300 |
| AL157475 | | 0.0418432 | 10846 | Hs.318791 | Hs.35453 | 301 |
| Contig43868_RC | FLJ12810 | −0.0083923 | 10958 | Hs.115660 | Hs.115660 | 302 |
| NM_002804 | PSMC3 | 0.0481223 | 11082 | Hs.250758 | Hs.250758 | 303 |
| NM_002915 | RFC3 | 0.0297288 | 11084 | Hs.115474 | Hs.115474 | 304 |
| NM_004102 | FABP3 | 0.0062634 | 11108 | Hs.112669 | Hs.49881 | 305 |
| Contig40852_RC | | −0.0039597 | 11424 | Hs.287850 | Hs.20295 | 306 |
| Contig41097_RC | | −0.0240801 | 11428 | Hs.434229 | Hs.283077 | 307 |
| Contig53180_RC | | 0.0163097 | 11509 | Hs.293943 | Hs.293943 | 308 |
| D42044 | KIAA0090 | 0.032282 | 11543 | Hs.439200 | Hs.154797 | 309 |
| NM_006638 | RPP40 | 0.0186764 | 11652 | Hs.511756 | Hs.115823 | 310 |
| NM_014060 | MCT-1 | 3.32E−04 | 11677 | Hs.102696 | Hs.102696 | 311 |
| NM_014891 | HASPP28 | 0.0012986 | 11688 | Hs.278426 | Hs.278426 | 312 |
| NM_018948 | MIG-6 | 0.0185109 | 11731 | Hs.11169 | Hs.11169 | 313 |
| Contig51654_RC | | −0.0209143 | 12014 | Hs.110783 | Hs.110783 | 314 |
| NM_002221 | TTPKB | 0.0298442 | 12126 | Hs.78877 | Hs.78877 | 315 |
| NM_002461 | MVD | 0.0362217 | 12130 | Hs.252457 | Hs.3828 | 316 |
| NM_002611 | PDK2 | 0.0545401 | 12140 | Hs.92261 | Hs.92261 | 317 |
| NM_005542 | INSIG1 | 0.0157828 | 12209 | Hs.416385 | Hs.56205 | 318 |
| NM_005887 | DLEU1 | 0.0071237 | 12217 | Hs.344524 | Hs.20149 | 319 |
| NM_017644 | FLJ20059 | 0.0132037 | 12294 | Hs.246875 | Hs.246875 | 320 |
| NM_017955 | FLJ20764 | 0.0395443 | 12302 | Hs.34045 | Hs.34045 | 321 |
| NM_017975 | FLJ10036 | 0.0306823 | 12304 | Hs.21331 | Hs.21331 | 322 |
| NM_018300 | FLJ11015 | −0.0271833 | 12310 | Hs.305953 | Hs.305953 | 323 |
| NM_019610 | LOC56267 | 0.0269821 | 12324 | Hs.134460 | Hs.180378 | 324 |
| AB040964 | DKFZP434C211 | 0.0048844 | 12371 | Hs.17270 | Hs.17270 | 325 |
| Contig3794_RC | | 0.0229191 | 12610 | Hs.188199 | Hs.15220 | 326 |
| NM_001458 | FLNC | −0.0597353 | 12842 | Hs.58414 | Hs.58414 | 327 |
| AB037726 | KIAA1305 | −0.0361304 | 13165 | Hs.288348 | Hs.288348 | 328 |
| AF070559 | | 0.0753652 | 13178 | Hs.398111 | Hs.13413 | 329 |
| Contig30070_RC | | 0.0429576 | 13446 | Hs.380126 | Hs.177781 | 330 |
| Contig36836_RC | | −0.023683 | 13540 | Hs.163725 | Hs.163725 | 331 |
| Contig37066_RC | LOC56926 | −0.0298591 | 13542 | Hs.24983 | Hs.24983 | 332 |
| Contig42355_RC | | 0.0014493 | 13631 | Hs.55080 | Hs.55080 | 333 |
| Contig48716_RC | DUT | 0.0613151 | 13705 | Hs.367676 | Hs.367676 | 334 |
| Contig52675_RC | | 0.0259649 | 13750 | Hs.131887 | Hs.34359 | 335 |
| Contig53315_RC | | 0.0036247 | 13756 | Hs.180591 | Hs.180591 | 336 |
| Contig705_RC | | −0.0026919 | 13823 | Hs.257267 | Hs.257267 | 337 |
| NM_000542 | SFTPB | 0.0385978 | 13872 | Hs.512690 | Hs.76305 | 338 |
| NM_005328 | HAS2 | −0.0158281 | 14071 | Hs.159226 | Hs.159226 | 339 |
| NM_005954 | MT3 | 0.0129362 | 14091 | Hs.73133 | Hs.73133 | 340 |
| NM_014904 | KIAA0941 | −0.0243201 | 14194 | Hs.173656 | Hs.173656 | 341 |
| NM_016021 | LOC51632 | 0.0685857 | 14212 | Hs.184325 | Hs.184325 | 342 |
| NM_016485 | HSPC228 | 0.0141849 | 14224 | Hs.267288 | Hs.267288 | 343 |
| NM_016621 | LOC51317 | 0.0100728 | 14231 | Hs.104888 | Hs.106826 | 344 |
| NM_018451 | BM032 | −0.0298676 | 14264 | Hs.434229 | Hs.283077 | 345 |
| NM_020139 | LOC56898 | −0.0347826 | 14291 | Hs.124696 | Hs.124696 | 346 |
| AB002365 | KIAA0367 | −0.0119917 | 14333 | Hs.23311 | Hs.23311 | 347 |
| AB033080 | KIAA1254 | 0.0377759 | 14377 | Hs.259326 | Hs.82506 | 348 |
| AB033094 | KIAA1268 | 0.0560176 | 14378 | Hs.152925 | Hs.152925 | 349 |
| AF097495 | | 0.0116724 | 14421 | Hs.128410 | Hs.239189 | 350 |
| AL161983 | | 0.0386629 | 14573 | Hs.21415 | Hs.21415 | 351 |
| Contig20552_RC | FLJ12810 | 0.0213887 | 14722 | Hs.115660 | Hs.115660 | 352 |
| Contig25390_RC | | 0.0416451 | 14816 | Hs.12702 | Hs.12702 | 353 |
| Contig25794_RC | | 0.0300592 | 14827 | Hs.440913 | Hs.173374 | 354 |
| Contig27901 | | 0.0163827 | 14874 | Hs.380763 | Hs.60293 | 355 |
| Contig28149_RC | | 0.0463053 | 14886 | Hs.350388 | Hs.350388 | 356 |
| Contig45569_RC | | 0.0311039 | 15328 | Hs.22116 | Hs.22116 | 357 |
| Contig565_RC | | −0.0273416 | 15542 | Hs.371609 | Hs.26418 | 358 |
| Contig58556_RC | HN1L | 0.0163717 | 15562 | Hs.437433 | Hs.172035 | 359 |
| NM_000504 | F10 | 0.0481014 | 15687 | Hs.361463 | Hs.47913 | 360 |
| NM_001503 | GPLD1 | 0.041847 | 15762 | Hs.512001 | Hs.272529 | 361 |
| NM_002130 | HMGCS1 | 0.0261251 | 15803 | Hs.397729 | Hs.77910 | 362 |
| NM_002340 | LSS | 0.0114226 | 15812 | Hs.442223 | Hs.93199 | 363 |
| NM_003762 | VAMP4 | −0.0259544 | 15905 | Hs.6651 | Hs.6651 | 364 |
| NM_003893 | LDB1 | 0.0020439 | 15919 | Hs.26002 | Hs.26002 | 365 |
| NM_004408 | DNM1 | 0.0468998 | 15958 | Hs.436132 | Hs.166161 | 366 |
| NM_004441 | EPHB1 | 0.0014544 | 15962 | Hs.272311 | Hs.78436 | 367 |
| NM_005177 | ATP6N1A | 0.0161722 | 16026 | Hs.267871 | Hs.267871 | 368 |
| NM_005204 | MAP3K8 | 0.0136797 | 16027 | Hs.432453 | Hs.248 | 369 |
| NM_005474 | HDAC5 | −0.0060292 | 16041 | Hs.9028 | Hs.9028 | 370 |
| NM_005687 | PheHB | 0.0290831 | 16054 | Hs.9081 | Hs.9081 | 371 |
| NM_006764 | IFRD2 | 0.0350204 | 16116 | Hs.315177 | Hs.315177 | 372 |
| NM_007366 | PLA2R1 | −0.0170268 | 16142 | Hs.410477 | Hs.171945 | 373 |
| NM_012268 | HU-K4 | 0.0086049 | 16163 | Hs.257008 | Hs.74573 | 374 |
| NM_015990 | LOC51088 | 0.0234822 | 16272 | Hs.272251 | Hs.272239 | 375 |
| AB002340 | KIAA0342 | 0.0238402 | 16480 | Hs.16950 | Hs.16950 | 376 |
| AB007867 | PLXNB1 | 0.0023466 | 16510 | Hs.278311 | Hs.278311 | 377 |
| AB033052 | KIAA1226 | 0.0660662 | 16619 | Hs.22151 | Hs.22151 | 378 |
| AJ225028 | GABBR1 | 0.0341089 | 16995 | Hs.167017 | Hs.167017 | 379 |
| AK001836 | LOC51088 | 0.0089177 | 17107 | Hs.272251 | Hs.272239 | 380 |
| AL110145 | | 0.0021157 | 17267 | Hs.483205 | Hs.432790 | 381 |
| AL110179 | | −0.0052602 | 17273 | Hs.208414 | Hs.208414 | 382 |
| AL110255 | | −0.020197 | 17284 | Hs.525899 | Hs.116808 | 383 |
| AL161961 | KIAA1554 | 0.0357458 | 17511 | Hs.195642 | Hs.17767 | 384 |
| Contig14968_RC | | 0.0031463 | 17871 | Hs.441130 | Hs.34359 | 385 |
| Contig27451_RC | | 0.0149366 | 18933 | Hs.150011 | Hs.150011 | 386 |
| Contig31351_RC | | 0.043506 | 19392 | Hs.405933 | Hs.333212 | 387 |
| Contig34363_RC | | 0.0218431 | 19737 | Hs.118630 | Hs.118630 | 388 |
| Contig34729_RC | | 0.0101084 | 19787 | Hs.442658 | Hs.17767 | 389 |
| Contig34962_RC | | 0.0184084 | 19817 | Hs.371609 | Hs.26418 | 390 |
| Contig35186_RC | | 0.0186019 | 19845 | Hs.334612 | Hs.334612 | 391 |
| Contig36275 | | 0.0800243 | 19975 | Hs.446406 | Hs.43628 | 392 |
| Contig36750_RC | | −0.0266751 | 20018 | Hs.167017 | Hs.167017 | 393 |
| Contig40877_RC | | −0.0198023 | 20436 | Hs.445410 | Hs.29088 | 394 |
| Contig40885_RC | | 0.0388978 | 20438 | Hs.44235 | Hs.44235 | 395 |
| Contig43133_RC | | 0.016295 | 20653 | Hs.126706 | Hs.126706 | 396 |
| Contig4324_RC | TUBA1 | 0.0209023 | 20663 | Hs.75318 | Hs.75318 | 397 |
| Contig43322_RC | | 0.0200381 | 20669 | Hs.115474 | Hs.115474 | 398 |
| Contig493_RC | | −0.0071587 | 21132 | Hs.236025 | Hs.79 | 399 |
| Contig49966_RC | | −0.027758 | 21171 | Hs.371609 | Hs.25144 | 400 |
| Contig56681_RC | | 0.0371499 | 21442 | Hs.301005 | Hs.301005 | 401 |
| Contig6089_RC | | 0.0033777 | 21515 | # N/A | Hs.388212 | 402 |
| Contig65995_RC | ZNF262 | 0.0191413 | 21596 | Hs.150390 | Hs.109694 | 403 |
| Contig8054_RC | | 0.0128438 | 21634 | Hs.357004 | Hs.94795 | 404 |
| Contig8896_RC | | −0.0352637 | 21682 | Hs.128410 | Hs.239189 | 405 |
| D29810 | | 0.0167239 | 21738 | Hs.173374 | Hs.173374 | 406 |
| NM_000059 | BRCA2 | 0.009552 | 21802 | Hs.34012 | Hs.34012 | 407 |
| NM_000245 | MET | 0.0309642 | 21838 | Hs.419124 | Hs.316752 | 408 |
| NM_000301 | PLG | 0.0015124 | 21854 | Hs.143436 | Hs.75576 | 409 |
| NM_000527 | LDLR | 0.0391406 | 21922 | Hs.213289 | Hs.213289 | 410 |
| NM_000757 | CSF1 | 0.0412441 | 21993 | Hs.173894 | Hs.173894 | 411 |
| NM_001289 | CLIC2 | 0.0360497 | 22101 | Hs.54570 | Hs.54570 | 412 |
| NM_001470 | GABBR1 | 0.0510091 | 22129 | Hs.167017 | Hs.167017 | 413 |
| NM_001798 | CDK2 | 0.0144224 | 22188 | Hs.19192 | Hs.19192 | 414 |
| NM_002167 | ID3 | 0.0491659 | 22261 | Hs.76884 | Hs.76884 | 415 |
| NM_002231 | KAI1 | 0.0386124 | 22277 | Hs.323949 | Hs.323949 | 416 |
| NM_002385 | MBP | −0.0155256 | 22317 | Hs.408543 | Hs.69547 | 417 |
| NM_002673 | PLXNB1 | 0.0308982 | 22375 | Hs.278311 | Hs.278311 | 418 |
| NM_002912 | REV3L | −0.0246321 | 22408 | Hs.232021 | Hs.115521 | 419 |
| NM_003174 | SVIL | 0.0029477 | 22460 | Hs.163111 | Hs.154567 | 420 |
| NM_003443 | ZNF151 | 0.0011354 | 22501 | Hs.433764 | Hs.33532 | 421 |
| NM_003632 | CNTNAP1 | −0.0127668 | 22533 | Hs.408730 | Hs.31622 | 422 |
| NM_003809 | TNFSF12 | 0.0254481 | 22560 | # N/A | Hs.26401 | 423 |
| NM_004655 | AXIN2 | −0.0110348 | 22720 | Hs.127337 | Hs.127337 | 424 |
| NM_004760 | STK17A | 0.009661 | 22741 | Hs.9075 | Hs.9075 | 425 |
| NM_004937 | CTNS | 0.0419382 | 22775 | Hs.187667 | Hs.64837 | 426 |
| NM_005451 | ENIGMA | −0.0025827 | 22920 | Hs.436339 | Hs.102948 | 427 |
| NM_006021 | DLEU2 | 0.0449619 | 23033 | Hs.446406 | Hs.43628 | 428 |
| NM_006328 | SIP | 0.0340722 | 23099 | Hs.11170 | Hs.11170 | 429 |
| NM_006271 | CRTAP | −8.15E−04 | 23107 | Hs.511747 | Hs.155481 | 430 |
| NM_006459 | KEO4 | −0.0177744 | 23123 | Hs.285818 | Hs.285818 | 431 |
| NM_006541 | TXNL2 | 0.0361709 | 23143 | Hs.42644 | Hs.42644 | 432 |
| NM_007086 | AND-1 | −0.0039346 | 23247 | Hs.385998 | Hs.72160 | 433 |
| NM_012301 | KIAA0705 | 0.0137393 | 23338 | Hs.22599 | Hs.22599 | 434 |
| NM_013402 | FADS1 | 0.029603 | 23404 | Hs.503546 | Hs.132898 | 435 |
| NM_014813 | KIAA0806 | 0.0174454 | 23600 | # N/A | Hs.24279 | 436 |
| NM_016336 | HSU93243 | 0.0247606 | 23760 | Hs.184325 | Hs.184325 | 437 |
| NM_018155 | FLJ10618 | −6.22E−04 | 23968 | Hs.144130 | Hs.42484 | 438 |
| NM_018283 | FLJ10956 | 0.0728296 | 23990 | Hs.144407 | Hs.144407 | 439 |
| NM_018677 | LOC55902 | 0.0176673 | 24113 | Hs.14779 | Hs.14779 | 440 |
| NM_020524 | HPIP | 0.0115252 | 24250 | Hs.505806 | Hs.8068 | 441 |
| U00952 | HPIP | 0.0333791 | 24362 | Hs.505806 | Hs.8068 | 442 |

| UniqID | Oligo Probe Sequence |
|---|---|
| NM_001615 | CAAGGATCCCCTCGAGACTACTCTGTTACCAGTCATGAAACATTAAAACCTACAAGCCTT |
| NM_001647 | CCCCCCATAAAGACAAACCAATCAACCACGACAAAGGAAGTTGACCTAAACATGTAACCA |
| NM_004265 | ACCCATAGGGAGCTGATCGTAATGTTTATCATGTTACTTCCCCACCCCTACATTTTTTGA |
| NM_001710 | CCCCTTGATAGTTCACAAGAGAAGTCGTTTCATTCAAGTTGGTGTAATCAGCTGGGGAGT |
| NM_014585 | ACGTAAAAGAGTGGTTAGTCACGTGAATTCAGTTATCATTTGACAGATTCTTATCTGTAC |
| NM_002345 | AAATAGGTGGTAGATATTGAGGCCAAGAATATTGCAAAATACATGAAGCTTCATGCACTT |
| AL133644 | ACTGAAGAAGGGATGTCCGCTATATCCAAAATTACAGCTATTGGCAAATAAACGAGATGG |
| AF113007 | GCTTGGTAATAAGAAGCACTTAAATCACTCCAAAGAAGACTTTAAAAAGGGAGCAGTGAA |
| NM_003878 | CAGATGGCAAGATTGAGTTTATTTCAACAATGGAAGGATATAAGTATCCAGTATATGGTG |
| Contig34896_RC | CTGGCTCATAGCATTTCACAAACATTATACTTCAGAGTCCCAAAGCCTTTAAATAAAATG |
| D25328 | TATTTTATCAGCACTTTATGCACGTATTATTGACATTAATACCTAATCGGCGAGTGCCCA |
| NM_002318 | GAGCACCATGTGTCATCACAGACACTTACACATACTTGAAACTTGGAATAAAAGAAAGAT |
| NM_020372 | TTGCTCTATCATTCTGTTTCAATAAAGACATTTGGAATAAACGAGCATATCATAGCCTGG |
| AL137274 | GGTACATTACTGCAATGTTCTCTTAACAGTTAAACAAGCTGTTTACAGTTTAAACTGCTG |
| Contig56705_RC | CAGCAACCTCAGACCAACCCAAGAAGATAATTTAAATCTATACTGCTTATTGGTCAATAT |
| Contig54295_RC | GTCACTCTACATGAATTATGTGCTCAAATTTGACCAACTCAGTTTAAGACACAAAACAGT |
| NM_000210 | GGAGGGTGGTCAACAAAGAAACAAAGATGTTATGGTGTTTAGACTTATGGTTGTTAAAAA |
| Contig57062_RC | GGCAGTTTACAGAACTCAATGACTTGTCATGAGGTTTTCATATGAGCTACACATTGTGTA |
| U95006 | TCCCCAGGCTATAAATTTGTTCTCACAAAGCAACATCAATAAATCAAAACTGTCTCTCCC |
| NM_005063 | AATAATGCTACCAGGATGCTAAAGATGATGATGTTAACCCATTCCAGTACAGTATTCTTT |
| NM_006074 | GAAAGAGAAACTTGTCAACTCATATCCACGTTATCTAGCAAAGTCATAAGAATCTATCAC |
| NM_004585 | TCATCAGTTCTGCGAAGGAGATGGTTGGTCAGAAGATGAAGTACAGTATTGTGAGCAGGA |
| NM_003129 | GTTGCAATCTATGCCGTGTATTTTTGCTTTAAGTCAGAACCTTGGATTACAAAACCTCGA |
| NM_016348 | ATCAAACAAAATCCTTGAGGTTGGTATACAAGTTAAGGCTGAAAAAAGGCCTTAAATTCC |
| AL133074 | TTTTTATACTGATTGGTTCATAGATGGTCAGTTTTGTACACAGACTGAACAATACAGCAC |
| NM_000076 | GTATTAATAACGTCTTTTTATATCTAAATGTATTCTGCACGAGAAGGTACACTGGTCCCA |
| NM_001993 | TGTAGGAAAGTAAAATGGAAGGAAATTGGGTGCATTTCTAGGACTTTTCTAACATATGTC |
| NM_003379 | TATGCATGTAATGGACTATGCACACTTTTAATTTTGTCAGATTCACACATGCCACTATGA |
| NM_000784 | CTAAAAGGCCACCCCTTTATCGCATTGCTGTCCTTGGGTAGAATATAAAATAAAGGGACT |
| X59405 | TTTATACCCGTTTCACATGCTTTTCAAGAATGTCGCAATTACTAAGAAGCAGATAATGGT |
| M55914 | GTACCGCTTCCTTAGAACTCTACAGAAGCCAAGCTCCCTGGAAGCCCTGTTGGCAGCTCT |
| NM_005694 | ATGGTGGTCTGCTGTGTGAATAAATAATTCCTGAAGAATGAAGAAGATTAATTTTGGGAG |
| Contig47563_RC | AGCAAACAATTAGCTGAAAACTTGGTATTGTTGTAGTTTATGTAGTAAGTGACTTGGCAC |
| AB040884 | GCACACTGTCAAATTCTTTTCCTTAAGGTGCACAGTAAATGTACAGATAGTTATAGGCCA |
| AF035284 | CAAAAAGACATGGAAGGTTAGGTTTGTCAACAGAAAGACATGGAAGATTAGAGGTTTGTC |
| NM_016423 | TTGGTACGATTCCTGTCATCCCTATTGCAGAGTCGTGTCCCCAATAAACAGGTGTCTTAA |
| NM_000484 | TTGGGTCTTTGATAAAGAAAAGAATCCCTGTTCATTGTAAGCACTTTTACGGGGCGGGTG |
| NM_001386 | ATTCCTTAAGAACTCTGTGTTATATTACCATGGAACGCCTAATAAAGCAAAATGTGGTTG |
| NM_003090 | TAATAGCCTTGTTTGTGTTAGCAAAGTGGAATCTATCAGCATTGTTGAAATGCTTAAGAC |
| NM_016058 | CACAAGAAGAAAGTATCGGGACATTATTGGATGCTATCATTTGTAGAATGTCAACAAAAG |
| NM_003255 | AGCTCTGACATCCCTTCCTGGAAACAGCATGAATAAAACACTCATCCCATGGGTCCAAAT |
| NM_005648 | TGTGCTATCGAAAGTATGCATGTATTTTACGTACAAGGTTCGCTACACTAACAGCTCCAC |
| AL050002 | CCGAGATATCTGTTTTGCAACAAGATGGGCTATATCTAAATAAAGACATGATCAAAGGAA |
| NM_000214 | GCTATTACGAAGTTCAAGATCAAAAAGGCTTATAAAACAGAGTAATCTTGTTGGTTCACC |
| AB029018 | GTGTTTCATATGCGTGACCGTTAAGATATTATCATTTAGGTGAAGGTTTCAACTCAAAAC |
| Contig37878_RC | AGACCTAACTGTTACCATCTGACTGAGGTTACTCTGGTATTTATCTCTTCGCTGCTGAAA |
| D43950 | TCTCTTGCAACACAAATGGTTAGAATGATTTTGAAGATTGATGACATTCGTAAGCCTGGA |
| NM_000366 | CGGAGAGGTCAGTAACTAAATTGGAGAAAAGCATTGATGACTTAGAAGACGAGCTGTACG |
| NM_004126 | AAGGAGACTTTCTTAAGCACCATATAGATAGGGTTATGTATAAAAGCATATGTGCTACTC |
| Contig2930_RC | ATGTGCTAAACTGGAGGTAACTATTTCTAGGTAGTTGAATTTTTGAAAGTCATGATCAGC |
| NM_000235 | TATAATTACTTTAGCTGCACTAACAGTACAATGCTTGTTAATGGTTAATATAGGCAGGGC |
| NM_001428 | AGCTCTAGCTTTTGCAGTCGTGTAATGGGCCCAAGTCATTGTTTTTCTCGCCTCACTTTC |
| NM_003670 | GACTCAGTTTTCAATTCCATCCTAAAACTCCTTTTAACCAAGCTTAGCTTCTCAAAGGCC |
| AK000060 | GAGAGAGAAAACATACCCACAGATAAAGACTCAGAATTTACTTCTTTGGGTATGAGTGCC |
| NM_003094 | GCGGATAGAAGGCTGTATCATTGGTTTTGATGAGTATATGAACCTTGTATTAGATGATGC |
| NM_002997 | TAGGAGAACCAAATCTGGAAGCCAAAATGTAGGCTTAGTTTGTGTGTTGTCTCTTGAGTT |
| NM_006813 | CAAGGTGAAGATCTAAATGTGAACAGTTTACTAATGCACTACTGAAGTTTAAATCTGTGG |
| NM_012385 | GCAGCAACAATAAATAGACACGCACGGCAGCCACAGCTTGGGTGTGTGTTCATCCTTGTT |
| NM_002444 | GCATTGCTGTGAATTAGCTCACTTGGTGATATGTCCTATATTGGCTAAATTGAAACCTGG |
| U82987 | GATGGCCCAGCCTGTAAGATACTGTATATGCGCTGCTGTAGATACCGGAATGAATTTTCT |
| AB007916 | GCAGCCGTGGTAGTAGCTGTCTATGATTCTTGCTCAGCAAAGTAAAATAAATGTTAAATA |
| NM_003289 | CAGAGGAGGAGTATTCCACCAAAGAAGATAAATATGAAGAGGAGATCAAACTGTTGGAGG |
| NM_004911 | ATAATTCTCTGTACAGGGGGGTTTGTGCTATACACTGGGATGTCTAATTGCAGCAATAAA |
| NM_000877 | AGGAAGAAGACACATTCCTAGTTCCCCGTGAACTTCCTTTGACTTATTGTCCCCACTAAA |
| NM_006519 | TTCATTTCAAAGGTGCTAAAATCTGAAATCTGCTAGTGTGAAACTTGCTCTACTCTCTGA |
| Contig57494_RC | TGCGCAGCACTTAACCAGTCGTTTGTATTCCCTTTCTTTCAATCCAATAAACAGAATGAA |
| NM_000269 | GTCTGAAATTCATGCAAGCTTCCGAAGATCTTCTCAAGGAACACTACGTTGACCTGAAGG |
| NM_018048 | GAAAATACATTAGCAAGCTTAATGGTTATCCTTACTTGAGTCCACATGGGTTGGACAGTC |
| Contig3607_RC | AAGCTGTGCTGTTTAGAAACAACATCTCAGACTTTACAAAGAAATGACAAAGAAGGCAAT |
| NM_006022 | ACCCCTTTTAAGAATTTGGCACAGTTACTCACTTTGTGTAATCTGAAATCTAGCTGCTGA |
| Contig3820_RC | CTGGTTACGAAGAAAACTCACCAATCATCTCCTTCCTGTTAATCACATGTTAATGAAAAT |
| Contig3695_RC | ATACCTCATTCTGACACCTGCATATAGTGTGGGAAATTGCTCTGCATTTGACTTAATTAA |
| NM_003186 | CCGTGGAGATCCCAACTGGTTTATGAAGAAAGCGCAGGAGCATAAGAGGGAATTCACAGA |
| Contig48249_RC | AGACGTGCCTTTAAGCAATAAAAATTCCAAGAGCTGATCATTATTGTGCTTCCATTTTAG |
| AK001362 | GAAACATCATGTCACATGAAACGATTCTCTGCTTTTTGGTTCTGAACTTGAAGTCCCTAA |
| NM_000222 | TTGACTTCAATGATAGTAAGAAAAGTGGTTGTTAGTTATAGATGTCTAGGTACTTCAGGG |
| NM_004107 | TCTGCCTCAGTTTCCCCTCCTAATACATATGGCTGTTTTCCACCTCGATAATATAACACG |
| NM_018455 | CCGACAAGAGGAGATCATTTTAGATATTACCGAAATGAAGAAAGCTTGCAATTAGTGAAC |
| AL049471 | GATTCCAGCTATGTAACCTCTATGCTCTGTAAGGTGATTATTTGTATATAGCAACATGGC |
| NM_000177 | ATCCAAAAATAGCCCTGCAAAAATTCAGAGTCCTTGCAAAATTGTCTAAAATGTCAGTGT |
| NM_001948 | GAGGTTTTGGTTCCACTGGAAAGAATTAAAATTTATGCCAAGAACAGAAAACAAGAAGTC |
| 590469 | GTGATTTCCAGTGAGTGTAAATAATTTTAAATAACCTCTGGCCCTTGGAATAAAGTTCTG |
| NM_014463 | TTCCACTCCTGAAATGAGTTGATTTGCAGATAACTCACAACTTCTTAAGCTAAATGGTAT |
| NM_016824 | ATTTTTGGAGTCCCATTGTTTCAGTGGGCATTAACAGAATGCTTTAAAAACTTCTAAGAC |
| NM_002106 | CGAGTCTTAACCATATTTAAGTGTTACTGTGGCTTCAAAGAAGCTATTGATTCTGAAGTA |
| Contig52945_RC | GGCAATCATTTTCAGACCACTATGTGTTTGAATCCTCTGGTATCAATACGTATTATAGGG |
| NM_001070 | GCCTGCAAACACATTTACTTCTCCTCTTATGAGACTATTTATCTTTAATAAAGCACTGGG |
| NM_006807 | GAGTCATGAGAACCATCAGTTCTTGATATTGTCTAGACTTGCATCTAGAGCTACGTTGTA |
| NM_002950 | AAGGAATTTTCAATATTTGATTGGTATTCTGTTCTGAAGTCTAGGATATTTTTCAGCCTA |
| NM_002792 | TATACTGACGAAGCCATTGAAACAGATGATCTGACCATTAAGCTGGTGATCAAGGCACTC |
| NM_004595 | ATTTTACACTGTTTGGAAGAAAGCTAAACCCTGAAGATCAGTAGCCCCTAATCACATGTG |
| NM_006745 | GAATTCACCTAAAGATCAAAATATCATGGATTGAACCTCATCAATTGATAGCAGTGAGTG |
| Contig1998_RC | TAAAAGTAACCATTGGAAACCTCGAATGAGGGCTAAAGTTTTAATCATAAGAGAAAAGGC |
| NM_002816 | AAATGTGGAGAAGATAACACTTGATTCCATTTCATTGTCATTAGTGTATTAACCAGCAGG |
| NM_006291 | GATATTGTTAATATAATAATAATTATTTTTTAGAGTACTGCTTTTGTATGTATGTTGAAC |
| NM_005962 | TGCAGCATTTGATAATGATAAAACACCTCACACCTCACTCTTTATAGTGCACAAAATGAA |
| AF052159 | GTTGGCATTGATATGGTACAACCTGCAAATTACTTGCAGTTCTGAGTTTCAGATAAAACA |
| NM_012412 | GTAATGCTTCTAAGGATCTCAAAGTAAAGCGTATCACTCCGCGTCACTTGCAGCTTGCAA |
| NM_016951 | ATTCATGCTCATCGTATCTGTGTTGGCACTGATACCAGAAACCACAACATTGACAGTTGG |
| NM_018639 | TGAACAGTTGCAAAGATGTCTTAATTGTGTAAAGAATTGGTGTAGTCATGACTTTAGCTG |
| NM_002166 | CAAAGCACTGTGTGGCTGAATAAGCGGTGTTCATGATTTCTTTTATTCTTTGCACAACAA |
| NM_006114 | CATCTCCTCGGTATAAATCATGTTTATAAGTTATGGAAGAACCGGGACATTTTACAGAAA |
| Contig58471_RC | TGGGCACTGTGGTTTTATTTCCTAATTGATTTAAGAAATAAACCTGAAGACCGTCTGGTG |
| NM_004457 | TTACCACCTATGACTGTACTTGTCAGTATGAGAATTTTTCTGAATCATATTGGGGAAGCA |
| AK002174 | GTTTTGCCACAGTTAACCCATTGTGCTTCTTTGTAATCAAACAGTTTGTGGGAGAATGGG |
| Contig2652_RC | GAGAACTAGATAGCATTGAAGCTGAACTTACAAGAAGAGTAGACATGATGGAACTGTGAC |
| Contig56768_RC | CATCTCATTGTACAGTGTTTTAGTTGCAAGCAGAAAGTAGAATTTGGTATAAAGCAGGTT |
| NM_007043 | GAGACAAGAGGAAAGAAACAAAGCATTTATTCCACCTAAGGAAAAACCAATTGTGAAACC |
| AF052100 | CCTTTGTGTTAATATCAGAAGTGTATTTGTAGCCCCTCCATAGTGAACAATGAAATAAAC |
| NM_000791 | AAACCCATGAAGGTAACTAACGGAAGGAAAAACTAAGAGAATGAAAAGTATTTGCCTCTG |
| NM_001424 | ATATGTACCAACAACACGAATTGCACAGTCATCAATGACAGCTTTCAAGAGTACTCCACG |
| U47101 | TGATTTGGGGGGAAATTACCAGTAGAATGCCTTGGTCTGAATATTTGATAGAACCAATTG |
| Contig4574_RC | ACAGACACCAGATTTGTGAATAAAGTTGGGGAATGGACAGCCTAACTGGGACATTGCAGT |
| NM_014018 | TGTGGAGAATGATCTGTACATAGATTTTGGTGGAAAGTTTCATTGTGTATGTAGAAGACC |
| Contig1505_RC | CTGAAACAGAAATGAAACTGTCCTTTTGACAACTCTCTTATATAATAAAGTATCACCGGC |
| Contig45316_RC | TCATACAATTTAATTGCTCAACCATGCATTTAAAACTCCTCAAGAAAGGATTGGTACTGC |
| NM_018281 | GCCAGATTGCCTTCATCATTTCACCTCTCCAGACTTCCATTTCTTCACAAGGATGATGAT |
| NM_003944 | ATGCTGCAGGTTGATGTAGACACAGTAAAAGGAGGGCTGAAGTTGAACCCCAACTGCCTG |
| NM_017455 | CTGTGACTTTAATAAGCTGGAACAGTCCACTGAATGGGTATAATGAATTGCAGTATATAC |
| AB029032 | AGGTTCTGTCAGTTCTTATCAAAAAGCTCGGTACTGCACTACAGGATGAAAAGGAAAAGA |
| Contig37141_RC | CTTTGCTTTGATTGAAGGCTGTAGAGCTGAGTTACCAAAATTTCTATTTCAAAGGAAACC |
| NM_004906 | GAGAATCAAATAATAGATGTCCGTACAAGTAGCGCATATATTTAACCATTTAGTTTGGGG |
| NM_005167 | GTCACACACCAGCACTTTATACACTTCTGGCTCACAGGAAAGTGTCTGCAGTAGGGACCC |
| Contig50396_RC | AGTATAATCTGCTTTACAACTAGTATAGACCTAAGGTCATTTGCTTTCAATTAGAGGCTC |
| NM_003793 | GCAAGAACTCTGGGCTTGGGTAATGAGCAGGAAGAAAATTTTCTGATCTTAAGCCCAGCT |
| NM_006347 | AAGAGACCATGGAAGTGTCAGAGATTCAGAATCCAAGATTGTCTTTAAGTTTTCAACTGT |
| AB033034 | CCATGAGTAACTCTGACAGGTATTTTAGATCATGATCTCAACAATATTCTTCCAAAATGG |
| NM_001706 | GGCAGACACGGATCTGAGAATCTTTATTGAGAAAGAGCACTTAAGAGAATATTTTAAGTA |
| NM_016217 | AGACAGATATGTACCTTATTAGAGCACCAGAACTAATTTGCTAAGTCTTTTGTTTAGTCC |
| AL049949 | TAGACTTGAATCTACTCTAAACGAATATTTAATCCAACCTCACTACATTGTAGCTCAGTC |
| NM_001482 | ACATACACAGATCCTAAGTAGAACCAGGTAATTGTCTCTTTTTCTAATAAGGAATTTGGG |
| NM_001551 | TAAAGTGTCAAGTGATTAAGTGTGTATTTGTACCCTAGATGATATGAACCAGCAGTCTTG |
| NM_005360 | GCATCGTGTACTTACCAGTGTGTTCACAAAATGAAATTTGTGTGAGAGCTGTACATTAAA |
| NM_014454 | CCTAAATGAAAGTGTGTAAATTATAAGAAGCTGGCGATCTTTTGATATGCTGTTTCACAG |
| Contig52737_RC | ACCATGTAAAATGTTGTAGAGATAGAGCCATATAACGTCACGTTTCAAAACTAGCTCTAC |
| NM_006303 | GAGTCAGAGTCTTTTTATTTAGGCCAGTTGTCAAGTGTCAATAAAAGCGCATCATGTAAT |
| NM_012428 | ACTGGTTGATGATAGATTTTATAACCTAACGGTTCTCATGCGGTGCGTAATTGTAGATGC |
| NM_016103 | CGTTCAGGCTTACTCAGAGATTTGATTGCTCAACATGCATAACTTGAATTCAATAGACTT |
| NM_016065 | ATGCAGAAGCTACAGATACAGAGGCTACAGAAACATAAATGAGCTGACTTTAGTGAGCAT |
| NM_020188 | CTGGGTTGATACCTGAAAGAATCCTGTCTTATTTGGTCTCCATAATCCTTTGAATGGAAA |
| NM_002185 | CACTACACAGTCTGCAAGATTCTGAAACATTGCTTTGACCACTCTTCCTGAGTTCAGTGG |
| NM_000099 | CATGACCAGCCACATCTGAAAAGGAAAGCATTCTGCTCTTTCCAGATCTACGCTGTGCCT |
| NM_001152 | GTGAACAGGCATGTTGTATTCTATAACACAATCTTGAGCATTCTTGACAGACTCCTGGCT |
| NM_014669 | TTGACGAGTATCATAGTGGTCATATTGATAGAGCTTTTGATATCATTGAGCGCTTGAAGC |
| NM_003093 | ATATTACCTGTTCTGCTTCACCAGGAGATCATGCTGCTGTGATACTGAGTTTTCTAAACA |
| NM_003115 | GCCAACCATTTCTTCACTGTACCATTTCTGAGAGATGTTGTCAATGTTTATGAACCTCAG |
| NM_012333 | GCTGAGATGAGAAGAGGAATGAGCCATATATTGGGGAAAATCATAGTTTGTAGGTATAAT |
| NM_014909 | CACTTTCACACTTCATCTCATTCCTGTTGTCACTTTCCCCGAAACGAATAAAGTCTCCCC |
| NM_019903 | GAAGTATTATTATAATTCACCATAAACAGCTATCTGTCTGAATTACTTCAGGCCTTCTCC |
| NM_001286 | TATTTGAACCTTTGGAACTTGGGAGTTCTCATTGTAACCCTAACATGTGAGAATAAAATG |
| NM_018287 | GCAAATCACTGTAATGAGAATGGTACTGGAAAAATACTGAATAGACTTGCTAAATGGCAC |
| AL133555 | TAGCCCATTGCCAAAAGGTTTTACTGTCTTAAAGCTGTCTTTCTGAGATCTAATTCCAAG |
| NM_004508 | CTTGTAAATAGTATTTACCAGTTAGCAAAGTCTGTGTTTTCAGAATTACAGTGAGCACAG |
| NM_000062 | GTGACGACCAGCCAGGATATGCTCTCAATCATGGAGAAATTGGAATTCTTCGATTTTTCT |
| NM_000373 | AAGAATGGGTTCTGGAGTTCTCATGGTCTTTAGGAAATATTGAGTAATTTGTAATCACCG |
| NM_021199 | TTCTGTAATTATGAAGAATCAAACACCAACAAAGAAGTATGATGGCTACACATCATGTCC |
| Contig54898_RC | ACAGAACGCAGGTTTTGGAATGGTCTTAAAAGATGTGAGGGTGTTAATCTAGGAAACTTC |
| NM_000788 | TCCAGACGCACTGATCTTTGCAAAGGAGACTTAATTTCAAATCTGTAATTACCATAGATA |
| NM_004301 | AAGTATGCGGTTGAAATTGATTGCAAATAATACAACAGTGGAACGGAGGTTTAGCTCATG |
| NM_004593 | TTGTCGGCTTTTATGTAATCTGTAATATGTATAGCAGGAAATACGAAGAGTTACACAGTG |
| NM_005517 | ATAGACTTAACTCCCTTAAGCCCAGACATCTGTTGAGACCTGACCCCTAGTCATTGGTTA |
| NM_017838 | ATTTGTCAACAAAGGAGAAAAAGGGATCATGGTTTTGGCAGGAGACACACTGCCCATTGA |
| NM_018243 | CTTGCTTAGTCTCCTTTCAGTATTTGGCAATAAAAGAAAGAAGAAATAGAACAGCTGAAG |
| Contig55612_RC | GTAAGGAACTTGGGTGTTAATAGTTGAGAGCTGTTTAGTAATAACCCAGTTTTCTTGAGG |
| Contig40105 | GCAGGAGTAGAAAGATGCTCATAGCACACGCGATATGTATGATAACATATTTTGTTTCAT |
| NM_003091 | TTCAGAAAGATCAAGCCAAAGAACTCCAAACAAGCAGAAAGGGAAGAGAAGCGAGTCCTC |
| Contig46583_RC | ATGCATAATAACCCAGTTTGTATCAAAGGGTATCGACTTAAGTGAAATTTCAACATGCTG |
| NM_012321 | GTGAAGCCCCTTTTTCTTGCTAAAACCGGCAATTCTCCGGTTAGAAATGTTACTTGGTGT |
| NM_014905 | GCAGGGACTGAATGACCTGATGTCAGATTTAGATTCTTCCTGGGGATTACACAGCTATGA |
| NM_016639 | CGCTGGCTCACACAAAACAGCTGACACTGACTAAGGAACTGCAGCATTTGCACAGGGGAG |
| Contig52717_RC | AAGTGCAAATGCCTCTTTGAAGCAATTCAGGCTAGGTAAACCGATTTTGCCATTTCAAAA |
| Contig58129_RC | TCATACATGGTATACAGATAGCTCATAATGAAGTCCAGAATCTTACTTTTAAGTGAAGGC |
| AK002107 | ACACAGTAGCAAAAGAGAAGATCTCATTTACAAATATCTATGGTGTTTCCTTGTTCTGTG |
| NM_002687 | CTTCAGGACTAGAAAGAAGTCACAAATCTTCAAAAGGTGGTAGTAGTAGAGATACAAAAG |
| NM_0028008 | CCCCCATTCTGGAAGGTTTTGTTATCTTCGGAAGAACCCCAATTATGATCTCTAAGTGAC |
| NM_014325 | TTTGATGACTGTACAACAGGAAGACTTGAAAAATCACGTGGATTCATATTACCACCGCTC |
| AL110212 | AGAGTCAGGAATACCATGTAACATGTTGAAGTAGACTAAAGATTAGTTCTTTGGCAATAG |
| NM_003374 | TCATATTGAGTAAATGAATGAAATTGTGATTTCCTGAGAATCGAACCTTGGTTCCCTAAC |
| L48692 | AAATACTTTACAGTGGTCGGTCACAAGAAACCATCTGAACAATTTCAGTCATTTGAAGCT |
| NM_001745 | TAGGGGTTGTAAAGCTACTTTATTAGATATAGAATGGCAGATTCTCTGATTTAAAAGGGC |
| NM_002659 | CTGGTAGCCACCGGCACTCACGAACCGAAAAACCAAAGCTATATGGTAAGAGGCTGTGCA |
| U09579 | CTTTGATTAGCAGCGGAACAAGGAGTCAGACATTTTAAGATGGTGGCAGTAGAGGCTATG |
| AL050353 | GGAAAGCTTGTCTGGGTTCTATACTGTGATCTCATTTGCCTCGACTACGATGGAAACATT |
| NM_003707 | ACAGCATTGAGAAAGAGCATGTCGAAGAGATCAGTGAACTTTTCTATGATGCCAAGTCCT |
| NM_006397 | CGAGAATCAGGAGGGACTCAGGAAGATCACATCCTACTTCCTCAATGAAGGGTCCCAAGC |
| NM_020143 | ATTTATCATTTATCTGAAATCACATGTAGCAGATTGCATAGTCTGTAATCCTCTCAGAGG |
| Contig38493_RC | AGGTGGTGATCTGAATACAGCAGCAGTTTGAAAGTGTTCCGTTTTTAAATAAACAGTATG |
| NM_002137 | GAGGATGAGAGCCCAGAGGTAACAGAACAGCTTCAGGTTATCGAAATAACAATGTTAAGG |
| NM_005805 | CAAGCCATCTATCCAGGCATTAATTCATGGACTAAACAGACATTATTACTCCATTACTAT |
| NM_014350 | AGAAAGGTAACAATCTTCATTCTACAGATGAACTCATTGAAACAATTTAGGGGAATGAGG |
| NM_018087 | AGATGGGACAGTAAATGTTCAGCATTCTTGGATCAGAAGAAAACGGACTAATTAGATGCT |
| AF113020 | AGATCCAGAACATGGGAAGTTAGGGAAAATGTGTGATTTTGTGTTTTGAATTACTGTCAG |
| AK001163 | CAGTGACTACGAGTAGTTCTTTCTCTATTGAATTATTAGGTCCAGAATAGAAGATGTCAT |
| Contig57034_RC | GCAAAAGAGTATTAATCCACTATCTCTAGTGCTTGACTTTAAATCAGTACAGTACCTGTA |
| NM_002431 | CGATAAGCCTCATCTGATGGAAGAGAGGAATAAATAATTCACCTATATGTGTTTGAGGTT |
| NM_004461 | CAGGGACAGAGGACTGGGTAGCAGGTTCCTTCTGTTGTCCTGTGTGGTGTGTCTACTGTG |
| NM_007107 | CAGAGATGCGACCTACTCAATCTGACTTAGTAAAACCATGCTGTAGAATTTTTGTCTTAA |
| U09848 | TATGACTTAAACCAACTACAACTTCCCTATAGCTTCTAAGCAGTTTCATCAGCATTACTT |
| NM_020401 | CTGCCAATGTTGTGTTTTCTGCTTCATACGATATTGCACAGTACTGGTCAGTATCAGGAA |
| NM_016271 | GTCACAGTGCTAAGTTATCTAGTTGGCTACTATTACACCTTAAAAATTGAGTTTACACAC |
| NM_002452 | GACGACAGCTACTGGTTTCCACTCCTGCTTCAGAAGAAGAAATTCCACGGGTACTTCAAG |
| NM_018290 | AAGGGCCAAATGATTCAAAACATCACAGGTATTTATGTGTTTTACAAAGACCTACATTCC |
| X66087 | AAACCAATGATTGTAGCAAACTCATACTGGATCATTTCAGTTACCTTGAACTAATAGCAC |
| Contig20651_RC | GGAGGGAATTGTATTGATACTCCAATACCTAAATTTCACAATACCACTTAGGAAAGCTAT |
| NM_016491 | CCCATCATCGATCTTCATGAATGCAATATTTATGATGTGAAAAATGACACAGGATTCCAG |
| D80010 | ATTACACTTTCAAAGAGAATTCCCTTTGCAATTTTATGTTTGGATCACCACTGTAAGCAC |
| NM_006618 | AATAAGCTTTCTCAGAATGTTGGCAAATCACTTCAATCCTCAAATCAGTCTTCCTTGTGG |
| AB020681 | AGAACAGGCTTTTAGAAGATAAAAGCGACAAGAAGGAATCTGGTGAATTTTAGTCATCCC |
| Contig41864_RC | AAATGATGGCCAAAGTATTAGAAAGAAACGAAGAGCCACTGGAGATGGATCTTCTCCTGA |
| NM_002949 | GACAAAGTGAAGCTGATCAAGGAAATCAAGAACTACATCCAAGGCATCAACCTCGTCCAG |
| NM_005920 | CCACTTCCTTTCTTGTGCTTCGTGTCCTGTTGACGGTTACATTTGTGTATAATTATTATA |
| NM_003089 | CAACCTTGGCCACTTGAGTTTGTCCTCCAAGGGTAGGTGTCTCATTTGTTCTGGCCCCTT |
| NM_016134 | AAGACGCAGAAATGATGTCAAGAATGGCTTCTCATGGGATCAAAATTGTCATTCAGCTAA |
| NM_016326 | AAGGCCACGTGAAGATGCTGCGGCTGGTGTTTGCACTTGTGACAGCAGTATGCTGTCTTG |
| NM_005826 | CCCTCCAGATTACTACGGCTATGAAGATTACTATGATGATTACTATGGTTATGATTATCA |
| NM_012417 | CTTTTGCATGGGTTGATGAGTGGTATGACATGACAATGGATGAAGTCCGAGAATTTGAAC |
| NM_016567 | GGGAACCTTCATGACTGTTGGAATTGCTCTGTCATAATAAGTCAGGGATATTTAGGGGGC |
| D55716 | ATGTGAATGAAGCCATCAGGCTAATGGAGATGTCAAAGGACTCTCTTCTAGGAGACAAGG |
| NM_004953 | CCTCCCCTGGGGCACAGAGATATATTATATATAAAGTCTTGAAAATTTTGAAATTTGGTG |
| NM_006824 | TGATGAATGCTATTAAGAAATATCAGAAAGGCTTCTCTGATAAACTGGATTTCCTTGAGG |
| NM_014413 | AGTTGTAGTGAATTGCTACTGAAAGCTATCCCAGGTGATACAGAGCTCTTTGTAAACCGC |
| NM_019555 | TACAAGTTTATTTAAAACTGCTTTCTCAAGTCGTTATTGATACAGCAAGTGAACCTGCTG |
| AB033054 | TGATAATATATTCTGCACGGTAAGAATTCCTTTTACAGACATTCTTTATCAAGAGGTCGG |
| NM_004095 | ATCTATGACCGGAAATTCCTGATGGAGTGTCGGAACTCACCTGTGACCAAAACACCCCCA |
| NM_006833 | CCATCACCAAAACGTGCAACACCATGAACCAGTTTGTGAACAAGTTCAATGTCCTCTACG |
| NM_012325 | CAGTGATTAACAATGCCAAAAAATGCAAGTAACTAGCCATTGTTCAAATGACAGTGGTGC |
| NM_016183 | TTTTTATTTGTCTGTAGACAGGGAACATGATGGGCACTGACCTCCTGTAAAGAATAAAAC |
| A8040969 | CCCACAGCAATAAAAGCTTCCCCCTGATATCCATCCCTTTGTAGTTTGAACAAATATATT |
| Contig43506_RC | AGTGAAGAGTGAGTGAAAGGAAGAATTCAGTGAATACATTGATACCTTGATATTATCTGC |
| Contig719_RC | GTTCTTGAGGTAGAAATGTCTACAGTCAGTTGTTTCATCTAGCTTGCATCTTAAAACACA |
| NM_005956 | CAGGTGAATGGATTATTCTAAACAGATCACCATCCATCTTCAAGAAGCTACTTTGAAAGT |
| NM_006938 | GTGCCTCCTTTATTAAGGGGTTCTTTGAGAATAAAAGAGAAAAGACCTACTTTATTTGAC |
| NM_016310 | ACCCGCAAGGTAACAAATCGGAAGTACCCAAAACTGAAAGAAGTGGATGATGTGCTTGGT |
| NM_016546 | GCATCAAGAGTCAAGTTGCTATATGGTCAAAGGTTAAATTTATCTCTAAAAAATGGCAGG |
| NM_016607 | TCTGGGAATAGAAAGTCACCATGATTTTTTGGTGAAAGTAAAAGTTGGAAAATTCATGGC |
| X69111 | GGAGACTAAACCTGGTGCTCAGGAGCGAAGGACTGTGAACTTGTAGCCTGAAGAGCCAGA |
| AF052183 | CTGCCCTCTCAGCTTGTGAGACAACACAGGAGCCTTCTATAGTATGTTGATATGCTAGAT |
| AK000685 | AAACTTGTGTAGGGAATCCTGACTTTTAAAATGTGAGGGTATTTGGATCTGTGTTGAAAG |
| Contig3902_RC | ATTTTTTAGAAAATACACACTTTTCAGGAGAAACCTGAGCATGATTTTGGATTCTCCACC |
| NM_013300 | GGGAGAACATCTGATGTTAACTTGACAGTCTTGTCTCGTGTATTGAATTCGTGCCAAAGG |
| Contig47710_RC | GAGATAACCTAGCTCTTTATATCTTCCCTTTTAAATAGAAACAACTGTCTTGAGAAGCTC |
| NM_002004 | GGATCTTGTCAGATTCACTGAAAAGAGGTACAAATCTATTGTCAAGTACAAGACAGCTTT |
| NM_003051 | AAATGCTGACAAAAATATTTTCCTAGCATCAGTAGATTTCTGGCATATGTTTCTGCTAGC |
| NM_003064 | TTCAAAGCTGGAGTCTGTCCTCCTAAGAAATCTGCCCAGTGCCTTAGATACAAGAAACCT |
| NM_006452 | CATCTGCGCATAAAGGACCAGATGAAACTCTGAGGATTAAAGCTGAGTATGAAGGGGATG |
| Contig54752_RC | CACACACACACTTAGTCTTGTAATTTCAGGCCAGAAATTCTCAACACTATTTTGCATCTG |
| M94362 | AAGTTATTTATGGCCTGGGAAACAATTTGCATTTGTCCCCAAATACGCTTAGCTGTGTGC |
| Contig49652_RC | CTGAAAGTGATACTCTTGGATGTGATTTTGAATTTCAAGAAAGCATCCATTCTCTATCAC |
| Contig56840_RC | AGCCAAGGACCGATTCCAGGCACTTTCTGTAGCAAATGACTGTGAATTACGACTTCTCTT |
| NM_020185 | TGGGCCTTTCTCAGAAGACTGTAATGTACCTGAAGTTTCTGAAATATTGCAAACCCACAG |
| NM_007021 | AAAGGAAAAAGTTTCAGACAAGCAATTACCCAGTTTCCTTATCTATAAAATGGGGACATC |
| NM_014264 | CTCACCAAATGGTCAAACAACTAGGTATGGAGAAAATGAAAAATTACCAGACTACATCAA |
| AL133577 | ACTCGTGATCATTGAGAAAGTGTTTGAAACTTTCTCATGAAGTGTATATATAATGGCGTG |
| NM_003016 | CCGATTGCTCCTGTGTAAAGATGCCTTGTCGTGCAGAAACAAATGGCTGTCCAGTTTATT |
| NM_002466 | CTTGTCCTGAGGTGTTGAGGGTGTCACGAGCCCATTCTCATGTTTACAGGGGTTGTGGGG |
| NM_006988 | GTTATCTCATGAGAGCTGTGATCCTTTAAAGAAACCTAAACATTTCATAGACTTTTGCAC |
| NM_016391 | TATCAAGATACCCCAAAACAGATTCGGAGTAAGATCAACGTCTATAAACGCTTTTACCCA |
| NM_017787 | TTAAACTGACCACTTGGAAGAAACACCTTGGTATCTGTGGTTTTCTTGCCTTGTCCCTGC |
| NM_017816 | CCAGACAATGAAATAACCATCAAAAAGCTAAGGAAAAAGGTTTTAGCTCAGTACTACACA |
| NM_021019 | CAACTTTCCCATCTTGTCTCTCTTGGATGATGTTTGCCGTCAGCATTCACCAAAATAAAC |
| NM_002388 | TGACCCAAGTCTTTGCCTCTACTCCCTTAACAGTGTTGAATTCAACTGAAGGCGAGGAAT |
| NM_003132 | TTTGAGTTCATGAAACAGAATCAGGATGCCTTCGACGTGATCATCACTGACTCCTCAGAC |
| NM_004280 | GGCAACATCTGTCTAGTGTTGTCTTCATCAAGAACAGACTATATACTAATTCCCACTAGA |
| AB036063 | GCTCCTTTGTAAAAAGTTAAAGATTTGAAAGAGAATCTCATATTCCCGAGGCATTAGGAA |
| Contig20635_RC | GGGAGCATAAAACATCCTTGACAGGTAGAGAGAGTTTTGAATGGCTTTTGTTATTTAAAA |
| Contig51797_RC | AGCTCTTTGCTACTGCTATGGTAAGATATCAAATGAAAAACCTCTGTAAAACATTTCCAG |
| NM_002184 | TGAGGAAGATTTTGTTAGACTTAAACAGCAGATTTCAGATCATATTTCACAATCCTGTGG |
| NM_006534 | GAGCCAGTTAATTTTAAGAATTTCACACATTTAGCCAATCTTTCTAGATGTCTCTGAAGG |
| NM_016613 | GAATACGGTAAAATAAATGACTTTAACCAAGTAGCTATAATGGGACTTAGCACTGTATGC |
| NM_002971 | AGTTGGAAAAGGATAATACAGACTGCACTAAATGTTTTCCTCTGTTTTACAAACTGCTTG |
| NM_005785 | GTGCTCAGGAATTTGAAAACGCTGCTATACTTACTCTGGTTACTACATTTCTTCCACTCC |
| NM_017768 | TTACCCATGATAGGACTTTTGTGATATGGCTAATCTCAGTACACATTTCAACTTAAAACC |
| NM_018381 | TAATTCCTTCCCTATCTCCTTACCAAAGTACAAGTCACATCTTTCCCACCTTTTCTGCAA |
| U79458 | TTCTGCCTTTTGCTGGTGTTTCTGGAATTTGCTTTCCCTCACCTCTCACTTCCTTCTAGA |
| AF054996 | TTCGCAAACCAGGACGACTACATATCATTTCGGCACCATGTGTATAAGAAGACAGACCAC |
| AL079279 | CCCTGCTTAAATGGTGGAAAATGTGTAAGACCAAACCGATGTCACTGTCTTTCTTCTTGG |
| Contig24856_RC | TTGCTTTGTCTCCCTGGATATGGATTTCAGTTAAGTATTTTGTAACCCGTTACACTGTGT |
| Contig2493_RC | TTGAGCTGCATTCAGGAAGTGCGGGACATGGTAGGGGAGGCAAAAAGCCTTGGGCACTAC |
| Contig49757_RC | CATGCATTGGATGTTTTGCTAAATAACTCCTGTGGATTTAGGAATGTGTGCTAATAGCAA |
| NM_004596 | GCTTTAAGATCACGCAGAACAACGCCATGAAGATCTCCTTTGCCAAGAAGTAGCACCTTT |
| NM_006396 | ACGATCCTCCTCCAAGACAAACAGCGGAAAATCTACTGCGTGGCTTGTCAGGAACTCGAC |
| NM_007066 | CACCTTCGCACCGTGCCCAGGTACACTTTCAAGACACTGTAACCACAAGATGTTATTTAT |
| AB037784 | CAAATGATTCCGGATCTCTAAAAGGCTCTCTCAGATGAAAAGGGAGTAAAGGAAAAAAGA |
| Contig37368_RC | GCAGCTGTAGATACAAAAAGTCCTCTAATGGAGTACTGGAATGTTTCATTGTCTTCACAA |
| NM_005704 | ATATTTTGCTCACTATCCCTCCCCACTTGCTTCCCTGATATGTGCTCTGACTTCCCTGAA |
| NM_005796 | GTTCCTATTAAAGAACATCAACGATGCTTGGGTTTGCACCAATGACATGTTCAGGCTCGC |
| AB002321 | CATTTGGAGACAGAGCCATTTGGATATTTTCCCCTTGAACTTCTCCATGACCTGAAGCGT |
| AF055016 | GTGTTACCTCAATTTCAGAGTAAGTAGTGGTTGATTAGTAATGTAGTATACACTGGCAGA |
| Contig48842_RC | AATGACAGGAAGATGTTCTTGTTTGGAACCTACCTGACTAAGAATGGCTCAGAGATACCC |
| NM_001274 | GTTGGGCTATCAATGGAAGAAAAGTTGTATGAATCAGGTTACTATATCAACAACTGATAG |
| NM_002692 | TGTGCCCGATCTACTTGTCATTGCAGACAAATATGATCCTTTCACTACGACAAATACCGA |
| NM_006444 | TGTGGTGTCACTAAAAGAAGGTATGTTCAACAATGCAAACGTTCTTTTCAAAACCAAGTT |
| NM_014977 | AGCCAAGGGTCTTTCACATCACCTATCCCTACATACATACCAAATGGAAAAGTGGCCATC |
| NM_016126 | CTTGTAATCCAAAGTTACTTTGTACAGACCTTGAAGATTGAAAAAACACGTCTAAAGAGC |
| Contig53132_RC | AGAGGGAAGACATTAAGGGGATTGGGGACATTTGTTTCACACATCTGCAGTAATATGAGT |
| NM_000935 | CCTTGAAAATCAGATTCTAACTGATTGTATGCAACTAAGTATTTCTGAACACCTATGCAG |
| M31212 | CTTGTCTCTCTTGGATGATGTTTGCCGTCAGCATTCACCAAAATAAACTTGCTCTCTGGG |
| NM_005928 | CTCACTGTCCTGTTTTCTTAGGCACTGAGGGATCTGAGTAGGTCTGGGATGGACAGGAAA |
| AK001025 | GTACAGGATGGATTATGGAGAATTTGGTAATCTAGTCGGAATAATTATATTGTATTGGGC |
| AL049435 | CTAAGTCATACTACTGATGGAATGAGAATACAAACCAAAAACACTTTATTCAAGTCCAGG |
| AL157475 | TTCCTTACTTTGCACAGTGAACACAACTAACCACATTAATTCAGCTTTGTGAAGTCCCTG |
| Contig43868_RC | GATTCCTACCTCTCATGATTACTATGGAGATTGAATAATTGGTAAAATTCTCCTAGCTCA |
| NM_002804 | TCATCTTCATTGATGAGTTGGATGCCATCGGCACCAAGCGCTTTGACAGTGAGAAGGCTG |
| NM_002915 | ATGGAGGATGGATTGGAAGGCATGATGTTCTGACTTCTGTCAGTTATTCTTGCAAAGATT |
| NM_004102 | CAAGCCTACCACAATCATCGAAAAGAATGGGGACATTCTCACCCTAAAAACACACAGCAC |
| Contig40852_RC | CATCTGCTTAGCTTAGTTCTACAAACTTTTTCATTTTTAATGTGCAAAGAACAGGCGGGG |
| Contig41097_RC | CAATTGTCAGAGTACAACGTGATGGCAACAAACTCATAGAGTTTAATAATGGCCAAAGAG |
| Contig53180_RC | CCTAATTTTCTCTCCTGGTAGCTGAACAAAGGTCTAAATTAGCTTAACAAAAGAACAGGC |
| D42044 | GACAAACTCTCTTACTCCTTAAGACAAATGCTCACCTGATCAATATGGGGAAATAAGCTG |
| NM_006638 | GGCATAAAACAGGTTCAGAAGAATCGACAATGATGTCATATTTTTCCAAGTACCAAATTC |
| NM_014060 | TATTGAACCATGGCTTAATCAAATCATGCCTAAGAAAGATCCTGTCAAAATAGTCCGATG |
| NM_014891 | GGAAAGCAAAAGACGATGGCACATTGTCAGGAAAACGAATGCAGTCACTCTCCCTGAATA |
| NM_018948 | CAGAGTTCCCATACCTCCTAGACCAGTAAAGCCAGATTATAGAAGATGGTCAGCAGAAGT |
| Contig51654_RC | CCTAGCTGTCTTCTTGAACTTGGGACTCTCCTTTCCCAAGACTTCCATCACTAGCTCCTG |
| NM_002221 | CTCCTTGAAAGCTTTGCCTTTGTTTTGAACTTCCTTTCCCACTTGGTAGAAAGAGCCCAG |
| NM_002461 | TGAAGGGCTTTCTCACCCCAGCTCTGGCTATGCCCAGTTCTCTGAGAAAGGAGCTCAGTG |
| NM_002611 | ACTGTGGAAAGCCATGAGTCCAGCCTCATTCTCCCACCCATCAAGGTCATGGTGGCCTTG |
| NM_005542 | ACCAGACCTAATTTGCAAGTATTGGGTCTTCAAACTTCAAGTGCAATGTATTATGAAAAC |
| NM_005887 | CATTCATGCCAAGTATCTTTCCAGCATGTTTCTCCCATTTAGAATATCTAGCATGTAAGG |
| NM_017644 | TCCAGTTGAAGATTACTGGATGCACGTACAGAATACATTCAGCCGTCAGGTAATAACATG |
| NM_017955 | CTAGGTTGAAGTCTAGAATGAAAGAAATCTGAATCCATGTCATTCATAACCCCTTGATCT |
| NM_017975 | ACAAGAACAGGTTTATCGTGTCCAAAAACTCCACCATATTCTAGAAATATTAGTCAGTTG |
| NM_018300 | CTCACATTGTACAATATTGCAAAAATTCATGCTTGAGAGAAACAAAAACACTGAGAGTGG |
| NM_019610 | AACACCTGTTTTTATTCCCCTGAGATCTAAACCTGTTTATGGAAAAAGATGGTCTAGTTC |
| AB040964 | TCTTTTCCCCAACCTAAAACCAACCACCAGCATTTCACTACAGGACCAAATGGAAACCGA |
| Contig3794_RC | GATATGAGCTCCTCATAGAAACCAGACCTACTGTATTAGACAGTAACCTCTAACCTCACC |
| NM_001458 | TACAACCCCAGAGTTTTAAGGACTTGGAAAGGAAGCACAATCAGAGAAGAAAACAGCCCC |
| AB037726 | GTTGCCCTTAAACCATTTCTAGCTGTTAACCCTATCCAGAAAAATGATTGAGTGATAGCT |
| AF070559 | CAGCATAAACTCAGAGATGAAGACCTCTTTCGTTTAAAAGAGCCTGTACTGTTTGTGTCA |
| Contig30070_RC | TGGTATAGCAAGAAGGTATTCTGAGCATTATATCAATGGGGAAGTTATAGATCTTTTGCA |
| Contig36836_RC | CTTCCTAGCAATAGTCTGCATTTAAAGAAAGGTGTGTTCAATTCATCAGCTTGAAATTGA |
| Contig37066_RC | GAGTTTGTCTTCTACGACCAGCTGAAGCAAGTGATGAATGCGTACAGAGTCAAGCCGGCC |
| Contig42355_RC | TCTTTTCTATCTGATCCACATGGAGAGGTTAAAGGTTCAATTTCATGACCTCTATGCAGG |
| Contig48716_RC | TATCAGGGTTTTGGGTGTCACTTAGGTTTTGTCCATCAGATTCTGTGAGACACCAGGCAT |
| Contig52675_RC | CTGGAATACATTAAAGAAGCAAAAGAACCTCATACAAAAGTCATTGAGTAAATGACAGCC |
| Contig53315_RC | AGAAAGAAGCCTTTTTCATTAAGGATACAACCTATTTGTAGCTCGCACTTTAAAAGATGC |
| Contig705_RC | TATGTTTCTTCCAAGGAAACAGGAGAGACTGAATTAATAATTCTCTCTTTCCTCTTAAGC |
| NM_000542 | ACACACAGGATCTCTCCGAGCAGCAATTCCCCATTCCTCTCCCCTATTGCTGGCTCTGCA |
| NM_005328 | AAGGATTTTGGAAACTCAAGGAAAAGTTCTTTCAACCTATACAACCTAACTTATGGACTG |
| NM_005954 | CTGTGGTGAAGTGTTCCTGGTGTTCCCTTTCCCTGCTGACCTTGGAGGAATGACAATAAA |
| NM_014904 | GTTTTATCCATCAAAAAATGCACTCTCACAGTGGAGTTGAAGTCTAATACATGATACATG |
| NM_016021 | CTTATTGAGTACTCTGTTTCTACGTATGTAGAATGTATAGGGATAGAAGAGTTGAAAAGG |
| NM_016485 | AACATGATCAAGTCCTTCTATACTGCAAGTCTTTTGATAGATGTCATAACAGTATTGGAG |
| NM_016621 | AAACTAAAATAAGGAATAGAAAAGCTGTTTTTCAGGCTGACAGTCCAATTAAGGGTAGCC |
| NM_018451 | GACATGCTACCCTAATAATGAAATAGGTATCCTGGACAAAACAATAAAAAGGAAGATTGC |
| NM_020139 | TGCAAGAGAAGGTGCCAAAGTCATAGCCACAGACATTAATGAGTCCAAACTTCAGGAACT |
| AB002365 | CATTGTTTCTCAAAGCTCCTTGATTAAGAGAAAGAACAGAAATTTGCACAGAAGATAGTG |
| AB033080 | GCAAGCTCAGTCATACATGACAAAGTGTAATTAACACTGATGTTTGTGTTAAATTTGCAG |
| AB033094 | TAATGTAAAACTGGTTGAACAAAACCGATGCCTGATTGAATGAAAACAATTCTATGAGGC |
| AF097495 | ACTAGTATAAAACTGTAAATACTACTATAAGACATTGGCTTTTTCCAGACATGGATCCGG |
| AL161983 | CAAAACGTAGCTTAGCAAAGCTGTTACTAAACTGGAGAGATCCTTTAATACATAGAAATG |
| Contig20552_RC | CAGGGTATTCTTCCAGGAGATTTGACCAGCAAGTGGAAAAATACCATAAACCCTGCTGAA |
| Contig25390_RC | GGCTTATAGAGTTAGAAGTCAGTATTTCTTCTAATCTGAGGCTATGATCAGTCCCAGCTG |
| Contig25794_RC | AGATACTGTATCTGGCTGTACTATACTAACAGTGATTTGCCTGCATGTGTTTGATAGAGA |
| Contig27901 | TTGAATCTCTTGCCAAAAGACATGGGTAAAATATCTGCCTCTCTCATAGAGATTTTAAGA |
| Contig28149_RC | GTGAAGACAAGAATAAAGCCACTAGTAGAAGCATCGGATTAGACACAACCTCGTCACAAC |
| Contig45569_RC | TGTCAGCAGACATGTTCATCCGATGATAGTACTGCAGTTTTCTATTAATAATTTGCAGAC |
| Contig565_RC | CTAGATCGAAGCAAAACCGATTTTGCTTTCACCATTAAGGTTGCATTTTAATGCAGTTAT |
| Contig58556_RC | GATGCCAAAATTAGTCTTCTCAAAGCTTTGAGTAGAGTAAGTGTGGGAATAAGCCAGTTT |
| NM_000504 | GTCATCAAGCACAACCGGTTCACAAAGGAGACCTATGACTTCGACATCGCCGTGCTCCGG |
| NM_001503 | GAAATATCAGACACACTCTGCTCATTCATGTCTCCTTCCACAGTTTATTTCCTCGCTTCC |
| NM_002130 | TAAAATAACAGCAAGTTTATGTGATCTTAAATCAAGGCTTGATTCAAGAACTGGTGTGGC |
| NM_002340 | GAATGCTGTTTGTGAGGTGTCTACAGGGTTTATAGTAGTCTTGTGGACACAGAAATGCAC |
| NM_003762 | CAGGTGGTGACTATCCTGTTGAGAAGCAAAAGATACTTTGCAAGTAAAAAATATGCCTCC |
| NM_003893 | CTTAAAGTTACGGGGCTTCGTTGAATGCTCCTTCCCCTAGGCAGCTGGTAATAGAGGGGT |
| NM_004408 | GCCCTGTTCTATAAATATCTATAAATACTCATATATATACACACCTACACATGGCCAACC |
| NM_004441 | GGTGATGTTCAACAGAAGTGAAGACAAAACAATATGCATCAGGAGAACAAGAGTAAACCC |
| NM_005177 | GGTTTCAAGTTCTTACCCTTCTCCTTCGAGCATATTCGGGAAGGGAAGTTTGAAGAGTGA |
| NM_005204 | AGCAATAAGCTGGACTAGTGTCCTAAAAATGGCTAACTGATGAATTAGAAGCCATCTGAC |
| NM_005474 | CTGATGTGGTCCTAGTCTCCGCCGGGTTTGATGCTGTTGAAGGACATCTGTCTCCTCTGG |
| NM_005687 | TGCAAAAAACTACAGACATCTCTGTGCTGTTTATTACAACAAGAATCCTGGGTTTGAGAT |
| NM_006764 | TCCAGTGAAAGTGTGAACCTGCGGATCGCTGCCGGTGAAACCATTGCACTGCTCTTTGAG |
| NM_007366 | ACGTTTTCTGAGAAAAAATAGCATGAAGCCTAAAGCCATTTCTTCCAAAAACAACATTGC |
| NM_012268 | CCACAACAAGTACATGGTGACTGAACGCGCCACCTACATCGGAACCTCCAACTGGTCTGG |
| NM_015990 | AGAAAGCCAGAAATTCCATCAATACATCTAGACAGATGTTTGCTTGTAGTTTTTGGTATC |
| AB002340 | TAGACTACCTCAGAATGTAGTCAAGAATTCACCCAGTGTTCTTTGGATGTCCCTTCCCTA |
| AB007867 | CATGAACTCTACAAGTACATCAACAAGTACTATGACCAGATCATCACTGCCCTGGAGGAG |
| AB033052 | CCATCTGAAATTGGATAGCCCACTGAAATTGAACATGCCTTCTCTTATAAATGTGTGGTG |
| AJ225028 | CCCATCTCCATTGCATTCATGTACTACCCTCAGTCTACACTCACAATCATCTTCTCCCAA |
| AK001836 | TGTCACCCAGTTCAGTACTTGGTACATAAAAGACAATAAATGTTTAACTGAGCCAAGATT |
| AL110145 | AGAAATAAGAATCAAGAGCAGCTATTGTTATCCTTAGAGTGTTTTCCGTCTAGGGCCGGC |
| AL110179 | AATTGCAATATTTGCAAACATGTGCCACAACAGTGTTCTTGGTGATGTTTCTAAAACAGT |
| AL110255 | GTAAATTCTTCAGGAATAAACTTCATGCTAGGTTAACATCAATGTATGGTTACAGCTTCC |
| AL161961 | GATTTACCTGAAAATCTTCACAACTGATCATTATCTCCTTCTCTTTGAGACCTGACTGAA |
| Contig14968_RC | CAAAATGAGTTTCAGCCAGAGTTTCAGTTTGACTTAAGTCAATTAAGCAACATCTCAAGG |
| Contig27451_RC | TTACATTATAAACTCTTTTGAAAAATGTATCTAAAATTTTTTAAGTTCTGTTTTGATTCC |
| Contig31351_RC | CAGTTTCCTCTCATCAAATCTAAAATTCTCCAAAATACTCTCAGGCATAACATACTTAGC |
| Contig34363_RC | TCCTTGATTGTGCTATGTTTCAGTGGAAGAAATTCTTTGAAGTAGATGTGAGTGAAAAAC |
| Contig34729_RC | TGAGATCCTGCAATGAGCAATCCTTTAAGCACTTTCTTCCCCGTTATGCTCCAAAGTACG |
| Contig34962_RC | AAATGACCACTAGAATAAAGAATTTTTCCAAACATCAGTGTCAAGCTCTTCCAATCAACC |
| Contig35186_RC | TTTCATGAAACTGAATAAAAGAAATCCTTTCCGAGATAGAGGAGATTTGTAGGTGAATGC |
| Contig36275 | CCAAAAGTCAAGAATTCAACCAGCAATTAAATAATAATTCCTGTGGTAAAGCACTTTCCC |
| Contig36750_RC | TCTGTACTTCCTAGACAACCTCCATTATTCCCTAAGGGAATCAGTGTTGTGTCTGTCTAC |
| Contig40877_RC | AAACAAAGGCACGACCAGATAAGTGACTTCAAGGGAGTAAAGGTCGTTCTATCGGAGAAC |
| Contig40885_RC | GGCTCAGAGAATCCTGCAAAATCCTTTTTTATTGTTTATTCTGCAGACAAAAATGTGACA |
| Contig43133_RC | TGTGAAAAGAAAACAAACAACTTGTACCTTCTTTCTGATATCACCGTCATTCCTCTTTCT |
| Contig4324_RC | CCCAGGGACTTTAAATTGAAGTCCTAGGGTTACTCAGTATAGGGAGGAAATGAGGACCAA |
| Contig43322_RC | TTCTCCTTCAAAACACTCATCTCCTCTGTAATGATCAGATGAACTCATGTCTTTCTCAGA |
| Contig493_RC | CATGCACATTGTCATGTGCAGCTTTGCATATACACACATGCATACATGAGCCTCCACACA |
| Contig49966_RC | ATGGACAAATAGCTGTGTAAATAGCTTCTCATAAAACGCTTCTACTGATAGCTGTTTGAC |
| Contig56681_RC | GTGCTCATTAATTCACGAGGCAGGATAAAATAGAAATTTCAAATTCTGATTGAATAGGCG |
| Contig6089_RC | GGAAGCTGAAATGGTACCATAAATGCTGCTAATCTGAGAGCCATTTTAAACTGCACTGCT |
| Contig65995_RC | CCCTCCATAATGAAAGAAGATAGGTTTATGCTTCTCTTTCCATTAATGTGCAGCCATTCT |
| Contig8054_RC | TTAATTGAAATAATGCTTAAAGGCTGTTGCTTCTCTTTAAAATTGACCCAAGGCATGAGC |
| Contig8896_RC | ACTTCGAAGGTATGTTTACAGGATGGATTAGCATGCACTTTACAGATATTTATGAAGTTG |
| D29810 | CTCCAAAAAATTTAGCCAAAGGTCGTGCCCCAAAATTTACGCAACCACTACAACCTCGCA |
| NM_000059 | AGTTTCAAATTTACCTCAGCGTTTGTGTATCGGGCAAAAATCGTTTTGCCCGATTCCGTA |
| NM_000245 | TGAACGTAAAATGTGTCGCTCCGTATCCTTCTCTGTTGTCATCAGAAGATAACGCTGATG |
| NM_000301 | GAGAATAAAGTGTGCAATCGCTATGAGTTTCTGAATGGAAGAGTCCAATCCACCGAACTC |
| NM_000527 | GCTTAAGAACATCAACAGCATCAACTTTGACAACCCCGTCTATCAGAAGACCACAGAGGA |
| NM_000757 | GCCAGACCATCTCTACCCTGTACTTGGACAACTTAACTTTTTTAACCAAAGTGCAGTTTA |
| NM_001289 | ACACAAAAGGAGGCAAATAAGAATTTTGAAAAATCTCTGCTCAAAGAATTCAAGCGTCTG |
| NM_001470 | GTACTTTCTTCCCTTAAATCATGGTATTCTTCTGACAGAGCCATATGTACCCTACCCTGC |
| NM_001798 | GCAAGATTTTAGTAAAGTTGTACCTCCCCTGGATGAAGATGGACGGAGCTTGTTATCGCA |
| NM_002167 | TAAAATGGATCCTGCACCACGGGAACCTCACAGCACCTCACTTCTTTTGGTTTTCTTTCT |
| NM_002231 | TTCGGATGGGCTGTTTAGATGTTATATAATCCACAAAAGGTTCATTGAGCTAAAAAAGTG |
| NM_002385 | GGCCAACAGTTAAATGAGAACATGAAAACAGAAAACGGTTAAAACTGTCCCTTTCTGTGT |
| NM_002673 | TGCATGAACTCTACAAGTACATCAACAAGTACTATGACCAGATCATCACTGCCCTGGAGG |
| NM_002912 | CAAAGTAGATCGTTATTTTGATCAAACTGTGCAAACAGTAGTACCACGTGTAGCATTTTG |
| NM_003174 | CAAAGTATTTTTCAATCAGAGTTTTCAGAACCTGACATTGTTAAAGATACTGCTTGTCCC |
| NM_003443 | GTGAAGCAAGTGCAGGAAGAAGACCCCAACACTCACATCCTCTACGCCTGTGACTCCTGT |
| NM_003632 | TAGCCAAAGCCATAAAAAACCTGCAACGTAGAGAAAATAATGCAGATACCCTGACTAGCC |
| NM_003809 | TGTTCACGTGTTTTCCATCCCACATAAATACAGTATTCCCACTCTTATCTTACAACTCCC |
| NM_004655 | GAGACTGCCATACATAATATATGACTTCCTAGGGATCTGAAATCCATAAACTAAGAGAAA |
| NM_004760 | CTTGGTCAGTGAAAAGATGCTACTATATTGCTTTTGTCCCAAAGTGAGTAAAATCCCCTA |
| NM_004937 | AGCGCATTAGCATAGTAACTCCTTTCAGATTTTTTGGAGGGACGTTTGGAAGTGGCTTAC |
| NM_005451 | GAGCGAGACTATGAGAAGATGTTTGGCACGAAATGCCATGGCTGTGACTTCAAGATCGAC |
| NM_006021 | AGCACGAATGATTCTATTAAAGAAAATCATTAGGAAGTGGTAGAAACTTTAAATCGCCCC |
| NM_006328 | TGTTAAGTGTTCGGCAGTAACCTACTTTGTTCCTTCGCCTCAGCAGCAAATCTTGCTACT |
| NM_006271 | TAGGCTAAATTTCTCCAGCCTCACAATGGTCTTCACTTGGTCTGACTTGTACCAATTCTA |
| NM_006459 | GGAACAATCATTATACGGACTCTTCAGATTTACAGAGAACTTACACTTCATCTGTTCCAC |
| NM_006541 | TTTGTCTGATATTTAGAGACATTCAGATTCAGAAGCCATGAGTTCAGTTAGCTCATGGCC |
| NM_007086 | CCTTGAAAAATCATTAAAGCCAAGGTATTAAAACCTTTGTGCATTAATACCTTCTAGGGG |
| NM_012301 | CTTAGGTTCCGTCTCACGGCGTTTTAATTTATTTTCACTGTCACACGCATAGATCCACGA |
| NM_013402 | TGACACAGATGAACCATATTCCCATGCACATTGATCATGACCGGAACATGGACTGGGTTT |
| NM_014813 | TTGCCCTAGTCTTCAGAATGGTCCTGAGAAAACATCACTACTTCGATGTTCTACTTTGCT |
| NM_016336 | CAGAATTGAAAGATCCAACAGATCATTACCATGCGCAGCTTTTAGAGGATAACCTTTTTG |
| NM_018155 | TCCAGTAAAATAACAGCAATGACAATGAGATCGTCAGTATTATTTTCACATTTCCCTGAG |
| NM_018283 | TCTAAACCAAGAGAAGTGAGACAAACCATCTAATATCTGTATCATGCTATTAATAGGCTG |
| NM_018677 | GAACGCTTTGAGACAACCTACTTTAAGAAGTTTCCTGGATACTATGTTACAGGAGATGGC |
| NM_020524 | GCTGTGCAACAGACAGGTGATGATGATGAAGTAGATGACTTTGAGGACTTCATCTTCAGC |
| U00952 | TGTCGTCGTCGAAGTAGATGACTTTGAGGACTTCATCTTCAGCCACTTCTTTGGAGACAA |
| Gene_symbol indicates the name of the gene, if known. Two identical names occur when more sequences of one and the same gene are present on the array. Centroid represents the mean expression (log 10 ratio) of a gene in a patient with local recurrence. Order_sig is the rank in significant expression on the total array (which contained 24.496 genes, indicated number is in the range from 1 (high) to 24.496 (low)). The last two columns indicate the UniGene duster notations, in | which 172 indicates the updated version. |

TABLE 2
Multivariate analysis (Cox-Regression) for
some clinical parameters and LR profile.

| | | | 95.0% CI for | |
|---|---|---|---|---|
| | | Hazard | hazard Ratio | |
| | Significance | Ratio | Lower | Upper |
| T2 [symbol] T1 | 0.873 | 2.044 | .274 | 15.276 |
| Grade III [symbol] I&II | 0.290 | 2.300 | .380 | 13.942 |
| Age <40 [symbol] >40 | 0.073 | 2.297 | .219 | 24.048 |
| CHT No [symbol] Yes | 0.115 | .330 | .063 | 1.731 |
| HORMONAL No [symbol] Yes | 0.308 | .259 | .039 | 1.703 |
| Boost Yes [symbol] No | 0.438 | .763 | .051 | 11.338 |
| LR Classifier + [symbol] − | 0.000 | 23.736 | 2.530 | 222.639 |

1) Tumor size: T1 vs T2 (<=2 cm or >2 cm)
2) Pathological grade III vs I-II
3) Patient's age at diagnosis
4) Yes or no adjuvant chemotherpy
5) Yes or no adjuvant hormonal therapy
6) Yes or no boost dose
7) Activated or quiescent Wound Signature

1. Method to predict a risk of local recurrence of breast cancer in patients having received breast conserving therapy, the method comprising the steps of:
a. measuring a wound signature gene expression profile of a patient; and
b. classifying said profile as “activated” or “quiescent”,
wherein a classification as “activated” indicates a high risk on local recurrence.

2. Method for determining a wound signature gene expression profile for local recurrence of breast cancer, the method comprising
a. determining a expression profile of at least the top two hundred of the genes listed in Table 1 in a breast tumor sample from at least one patient with local recurrence;
b. determining an expression profile of the genes in a breast tumor sample from at least one patient without local recurrence; and
c. determining from said expression profiles an “activated” and/or a “quiescent” expression profiles.

3. The method according to claim 2, further comprising:
a. hybridizing RNA or a derivative thereof obtained from a breast tumor sample to a set of nucleic acid molecules comprising probes for at least the top 200 of the genes listed in Table 1; and
b. quantifying the hybridization signals obtained from the RNA or a derivative thereof to the probes.

4. The method according to claim 3, further comprising determining a mean expression value for each of the hybridization signals to the probes.

5. A method for determining the risk for local recurrence in a breast tumor sample from a patient, the method comprising
a. determining the expression profile of at least the top two hundred of the genes listed in Table 1 in a breast tumor sample from the patient ;
b. comparing the profile with at least one wound signature gene expression profile obtained in the method according to claim 2, and
c. determining from the comparison whether the sample is of a patient at high or low risk for local recurrence.

6. The method according to claim 5, wherein a Pearson correlation of the mean expression value is used for comparing the profiles.

7. The method according to claim 1, wherein the wound signature gene expression profile comprises the expression profile of at least about 60%, of the 442 genetic elements listed in Table 1.

8. The method according to claim 1, wherein the wound signature gene expression profile comprises at least the expression profile of the top 200 of the 442 genetic elements listed in Table 1.

9. A method of determining a risk on local recurrence in a breast cancer patient treated with breast conserving therapy, the method comprising:
using a wound signature gene set for the determination of the risk on local recurrence in breast cancer patients treated with breast conserving therapy.

10. The method according to claim 9, wherein the wound signature gene set comprises at least about 60% of the 442 genetic elements listed in Table 1.

11. The method according to claim 7, wherein the wound signature gene expression profile comprises the expression profile of at least about 70% of the 442 genetic elements listed in Table 1.

12. The method according to claim 11, wherein the wound signature gene expression profile comprises the expression profile of at least about 80% of the 442 genetic elements listed in Table 1.

13. The method according to claim 12, wherein the wound signature gene expression profile comprises the expression profile of at least about 90% of the 442 genetic elements listed in Table 1.

14. The method according to claim 13, wherein the wound signature gene expression profile comprises the expression profile of at least about 95% of the 442 genetic elements listed in Table 1.

15. The method according to claim 14, wherein the wound signature gene expression profile comprises the expression profile of at least about 99% of the 442 genetic elements listed in Table 1.

16. The method according to claim 15, wherein the wound signature gene expression profile comprises the expression profile of all of the 442 genetic elements listed in Table 1.

17. The method according to claim 8, wherein the wound signature gene expression profile comprises at least the expression profile of the top 250 of the 442 genetic elements listed in Table 1.

18. The method according to claim 17, wherein the wound signature gene expression profile comprises at least the expression profile of the top 300 of the 442 genetic elements listed in Table 1.

19. The method according to claim 18, wherein the wound signature gene expression profile comprises at least the expression profile of the top 350 of the 442 genetic elements listed in Table 1.

20. The method according to claim 18, wherein the wound signature gene expression profile comprises at least the expression profile of the top 440 of the 442 genetic elements listed in Table 1.
